(19) United States
(12) Patent Application Publication
(10) Pub. No.: US 2024/0132563 A1
(43) Pub. Date: Apr. 25, 2024

(54) BIFUNCTIONAL CYTOKINE COMPOSITIONS

(71) Applicant: Bright Peak Therapeutics AG, Basel (CH)

(72) Inventors: Vijaya Raghavan PATTABIRAMAN, Volketswil (CH); Bertolt KREFT, Kleinmachow (DE); Jean-Philippe CARRALOT, Saint-Louis (FR); Philipp MOOSMANN, Basel (CH)

(21) Appl. No.: 18/113,403

(22) Filed: Feb. 23, 2023

Related U.S. Application Data
(60) Provisional application No. 63/313,248, filed on Feb. 23, 2022.

(51) Int. Cl. C07K 14/55; C07K 14/54

(52) U.S. Cl. CPC C07K 14/55; C07K 14/54; C07K 2319/00

(57) ABSTRACT

The present disclosure relates to bifunctional cytokine compositions comprising first and second cytokines connected by a linker, as well as methods of making bifunctional cytokine compositions. The disclosure also relates to bifunctional cytokine compositions comprising interleukins, including interleukin-2, interleukin-7, and interleukin-18, as well as derivatives thereof.

### CROSS REFERENCE

This application claims the benefit of U.S. Provisional Application No. 63/313,248 filed Feb. 23, 2022, which application is incorporated herein by reference in its entirety.

### SUMMARY OF THE INVENTION

Provided herein are bifunctional cytokine compositions. The bifunctional cytokine compositions provided herein comprise two cytokine polypeptides connected by a linker. In some embodiments, the presence of two cytokines in one molecule allows for enhanced modulation of the immune system or an immune cell compared to the cytokines individually or in combination. In some embodiments, the bifunctional cytokines comprise one or more chemically synthesized cytokine polypeptides. Also provided herein are methods of manufacturing bifunctional cytokine compositions, including methods which produce bifunctional cytokine compositions with a high degree of uniformity.

Further provided herein are bifunctional cytokine compositions which contain an IL-2 and an IL-18 polypeptide. In some embodiments, the bifunctional cytokine compositions comprises an IL-2 polypeptide which is biased in favor of the IL-2 receptor beta subunit (e.g., better binding and/or signaling for the alpha subunit relative to the beta subunit, such as by having substantially reduced affinity for the IL-2 receptor alpha subunit) and an IL-18 polypeptide which is resistant to binding by IL-18 binding protein (IL-18BP). In some instances, such bifunctional cytokine compositions are useful for the treatment of cancer.

In one aspect, provided herein is a bifunctional cytokine composition, comprising: a first cytokine; a second cytokine; and a chemical linker comprising a first point of attachment to the first cytokine and a second point of attachment to the second cytokine. In some embodiments, one or both of the first cytokine and the second cytokine are synthetic. In some embodiments, the first point of attachment is to a point which is not the N-terminal amine or C-terminal carboxyl of the first cytokine and/or the second point of attachment is to a point which is not the N-terminal amine or C-terminal carboxyl of the second cytokine. In some embodiments, one or both of the first point of attachment and the second point of attachment are at a pre-selected residue. In some embodiments, the pre-selected residue comprises a conjugation handle for attachment of the chemical linker. In some embodiments, the pre-selected residue is an unnatural amino acid or a modified natural amino acid residue. In some embodiments, the chemical linker comprises a chemical polymer, a bifunctional linker, or a combination thereof. In some embodiments, the chemical linker comprises a chemical polymer, wherein the chemical polymer comprises poly(alkylene oxide), polysaccharide, poly(vinyl pyrrolidone), poly(vinyl alcohol), polyoxazoline, poly(acryloylmorpholine), or a combination thereof. In some embodiments, the chemical linker comprises polyethylene glycol. In some embodiments, the chemical linker is attached to the first point of attachment and/or the second point of attachment through a reaction with a conjugation handle. In some embodiments, a bifunctional linking reagent is used to form at least a part of the chemical linker. In some embodiments, the bifunctional linking reagent comprises a conjugation handle complementary to a cysteine sulfhydryl of the first cytokine or the second cytokine. In some embodiments, each of the first cytokine and the second cytokine independently comprise from about 50 to about 300 amino acid residues, from about 50 to about 250 amino acid residues, from about 50 to about 200 amino acid residues, from about 75 to about 300 amino acid residues, from about 75 to about 250 amino acid residues, from about 75 to about 200 amino acid residues, from about 100 to about 300 amino acid residues, from about 100 to about 250 amino acid residues, or from about 100 to about 200 amino acid residues. In some embodiments, one or both of the first cytokine and the second cytokine is an interleukin. In some embodiments, the first cytokine and the second cytokine are different interleukins. In some embodiments, each interleukin is independently selected from an IL-2, an IL-7, an IL-10, an IL-12, and an IL-18. In some embodiments, the first cytokine is an IL-18. In some embodiments, the second cytokine is an IL-2 or an IL-7. In some embodiments, the first point of attachment of at least about 80%, at least about 85%, at least about 90%, at least about 95%, at least about 96%, at least about 97%, at least about 98%, or at least about 99% of individual bifunctional cytokine compositions is to the same residue position of the first cytokine. In some embodiments, the second point of attachment of at least about 80%, at least about 85%, at least about 90%, at least about 95%, at least about 96%, at least about 97%, at least about 98%, or at least about 99% of individual bifunctional cytokine compositions is to the same residue position of the second cytokine. In some embodiments, the population comprises at least 10,000 individual bifunctional cytokine compositions.

In one aspect, provided herein is a bifunctional cytokine composition, comprising an IL-18 polypeptide, wherein the IL-18 polypeptide comprises at least 2 amino acid substitutions to the sequence set forth in SEQ ID NO: 1; a second cytokine; and a linker comprising a first point of attachment to the IL-18 polypeptide and a second point of attachment to the second cytokine. In some embodiments, the IL-18 polypeptide exhibits reduced binding to IL-18 binding protein (IL-18BP) compared to wild type IL-18 (WT IL-18). In some embodiments, the bifunctional cytokine composition exhibits enhanced binding to an IL-18 receptor (IL-18R) compared to WT IL-18. In some embodiments, the bifunctional cytokine composition exhibits binding to an IL-18 receptor (IL-18R) which is reduced by at most 100-fold compared to WT IL-18. In some embodiments, the IL-18 polypeptide comprises at least one substitution at residue Y1, F2, E6, V11, C38, K53, D54, 555, T63, C76, E85, M86, T95, D98, or C127, or any combination thereof. In some embodiments, the IL-18 polypeptide comprises a Y01G, F02A, E06K, V11, C38S, C38A, D54A, S55A, T63A, C76S, C76A, E85C, M86C, T95C, D98C, C127S, or C127A amino acid substitution, or any combination thereof. In some embodiments, the IL-18 polypeptide comprises a Y01G, F02A, E06K, V11I, C38S, C38A, K53A, D54A, S55A, T63A, C76S, C76A, E85C, M86C, T95C, D98C, C127S, or C127A amino acid substitution, or any combination thereof. In some embodiments, the IL-18 polypeptide comprises E06K and K53A amino acid substitutions. In some embodiments, the IL-18 polypeptide comprises a T63A amino acid substitution. In some embodiments, the IL-18 polypeptide comprises a V11I amino acid substitution. In some embodiments, the IL-18 polypeptide is synthetic. In some embodiments, the second cytokine is an IL-2 polypeptide or an IL-7 polypeptide. In some embodiments, the second cytokine is an IL-2 polypeptide. In some embodiments, the IL-2 is biased towards the IL-2 receptor subunit beta (IL-2Rβ) compared to wild type IL-2.

In one aspect, provided herein is a bifunctional cytokine composition, comprising: an interleukin-18 (IL-18) polypeptide, wherein residue position numbering of the IL-18 polypeptide is based on SEQ ID NO: 1 as a reference sequence; an interleukin-2 (IL-2) polypeptide, wherein the IL-2 polypeptide is biased towards the IL-2 receptor subunit beta (IL-2Rβ) compared to wild type IL-2, wherein residue position numbering of the IL-2 polypeptide is based on SEQ ID NO: 301 as a reference sequence; and a linker comprising a first point of attachment to the IL-18 polypeptide and a second point of attachment to the IL-2 polypeptide. In some embodiments, the IL-2 polypeptide exhibits reduced binding to the IL-2 receptor subunit alpha (IL-2Rα). In some embodiments, the IL-2 polypeptide comprises an amino acid substitution at a residue which contacts IL-2Rα. In some embodiments, the IL-2 polypeptide comprises an amino acid substitution at residue 35, 37, 38, 41, 42, 43, 44, 45, 60, 61, 62, 64, 65, 68, 69, 71, 72, 104, 105, 107, or any combination thereof, of the IL-2 polypeptide, wherein residue position numbering is based on SEQ ID NO: 301 as a reference sequence. In some embodiments, the IL-2 polypeptide comprises a non-linker polymer attached at residue 35, 37, 38, 41, 42, 43, 44, 45, 60, 61, 62, 64, 65, 68, 69, 71, 72, 104, 105, 107. In some embodiments, the IL-2 polypeptide comprises a non-linker polymer attached at residue 42 or 45, or two non-linker polymers, wherein one of the two non-linker polymers is attached at residue 42 and one of the two non-linker polymers is attached at residue 45. In some embodiments, the non-linker polymer comprises polyethylene glycol. In some embodiments, the IL-2 polypeptide is synthetic. In some embodiments, the IL-2 polypeptide comprises an amino acid sequence having at least about 80%, 85%, 90%, 95%, 96%, 97%, 98%, 99%, or identical to a sequence set forth in SEQ ID NOs: 301-323. In some embodiments, the second point of attachment is at residue 1, 35, 37, 38, 41, 42, 43, 44, 45, 60, 61, 62, 64, 65, 68, 69, 71, 72, 104, 105, or 107 of the IL-2 polypeptide. In some embodiments, the second point of attachment is at residue 1, 42, or 45 of the IL-2 polypeptide. In some embodiments, the bifunctional cytokine composition exhibits reduced binding to IL-18 binding protein (IL-18BP) compared to wild type IL-18 (WT IL-18). In some embodiments, the bifunctional cytokine composition exhibits enhanced binding to an IL-18 receptor (IL-18R) compared to WT IL-18. In some embodiments, the bifunctional cytokine composition exhibits binding to an IL-18 receptor (IL-18R) which is reduced by at most 100-fold compared to WT IL-18. In some embodiments, the IL-18 polypeptide comprises an amino acid sequence having at least about 80%, 85%, 90%, 95%, 96%, 97%, 98%, 99%, or identical to a sequence set forth in SEQ ID NOs: 1-67. In some embodiments, the IL-18 polypeptide comprises the sequence set forth in SEQ ID NO: 30. In some embodiments, the IL-18 polypeptide comprises an amino acid sequence set forth in any one of SEQ ID NOs: 68-72. In some embodiments, the first point of attachment is to a position other than the N-terminal amine or C-terminal carboxyl of the IL-18 polypeptide. In some embodiments, the first point of attachment is in a region comprising residues 30-150 of the IL-18 polypeptide. In some embodiments, the first point of attachment is to a residue selected from residue 38, 68, 69, 70, 76, 78, 85, 86, 95, 98, 121, 127, or 144 of the IL-18 polypeptide. In some embodiments, the first point of attachment is to a residue selected from residue 68, 69, 70, 85, 86, 95, or 98 of the IL-18 polypeptide. In some embodiments, the first point of attachment is to residue 68 of the IL-18 polypeptide. In some embodiments, the IL-2 polypeptide comprise the sequence set forth in SEQ ID NO: 303. In some embodiments, the bifunctional cytokine composition induces STAT5 phosphorylation in CD4⁺ T cells, CD8⁺ T cells, and/or NK cells more potently than a corresponding IL-2 polypeptide. In some embodiments, the bifunctional cytokine composition increases p65 levels in NK cells more potently than a corresponding IL-18 polypeptide. In some embodiments, wherein in peripheral blood mononuclear cells, the bifunctional cytokine composition induces greater IFNγ production than a corresponding IL-18 polypeptide. In some embodiments, the bifunctional cytokine composition exhibits an EC₅₀ of IFNγ production which is lower than for a corresponding IL-2 polypeptide. In some embodiments, the linker is a chemical linker. In some embodiments, the chemical linker comprises from about 2 to about 100 ethylene glycol units. In some embodiments, the linker has a linear length of from about 10 angstroms to about 200 angstroms.

In one aspect, provided herein is a pharmaceutical composition comprising a bifunctional cytokine provided herein and a pharmaceutically acceptable carrier.

In one aspect provided herein, is a method for treating cancer in a subject, comprising administering to the subject a pharmaceutically acceptable amount of a bifunctional cytokine composition provided herein to the subject. In some embodiments, the cancer is a solid cancer. In some embodiments, the solid cancer is adrenal cancer, anal cancer, bile duct cancer, bladder cancer, bone cancer, brain cancer, breast cancer, carcinoid cancer, cervical cancer, colorectal cancer, esophageal cancer, eye cancer, gallbladder cancer, gastrointestinal stromal tumor, germ cell cancer, head and neck cancer, kidney cancer, liver cancer, lung cancer, nasal cavity and paranasal sinus cancer, nasopharyngeal cancer, neuroblastoma, neuroendocrine cancer, oral cancer, oropharyngeal cancer, ovarian cancer, pancreatic cancer, pediatric cancer, penile cancer, pituitary cancer, prostate cancer, skin cancer, soft tissue cancer, spinal cord cancer, stomach cancer, testicular cancer, thymus cancer, thyroid cancer, ureteral cancer, uterine cancer, vaginal cancer, or vulvar cancer. In some embodiments, the solid cancer is metastatic renal cell carcinoma or melanoma. In some embodiments, the solid cancer is a carcinoma or a sarcoma. In some embodiments, the cancer is a blood cancer. In some embodiments, the blood cancer is leukemia, non-Hodgkin lymphoma, Hodgkin lymphoma, an AIDS-related lymphoma, multiple myeloma, plasmacytoma, post-transplantation lymphoproliferative disorder, or Waldenstrom macroglobulinemia.

In one aspect, provided herein is a method of making a bifunctional cytokine composition, comprising: providing a first cytokine, wherein the first cytokine comprises a first cytokine conjugation handle; providing a second cytokine, wherein the second cytokine comprises a second cytokine conjugation handle; and forming a covalent bond through a reaction of the first cytokine conjugation handle with the second cytokine conjugation handle to form a linker.

In one aspect, provided herein is a method of making a bifunctional cytokine composition, comprising, providing a first cytokine, wherein the first cytokine comprises a first cytokine conjugation handle; providing a second cytokine, wherein the second cytokine comprises a second cytokine conjugation handle; providing a bifunctional reagent, wherein the bifunctional reagent comprises a first reagent conjugation handle and a second reagent conjugation handle, wherein the first reagent conjugation handle is complementary to the first cytokine conjugation handle, and wherein the second reagent conjugation handle is complementary to the second cytokine conjugation handle; forming a first covalent bond through a reaction of the first cytokine conjugation handle and the first reagent conjugation handle; and forming a second covalent bond through a reaction of the second cytokine conjugation handle and the second reagent conjugation handle.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A shows an exemplary IL-2/IL-18 bifunctional cytokine composition of the instant disclosure, as well as proposed mechanisms of action and signaling activation.

FIG. 1B shows an exemplary IL-2/IL-18 bifunctional cytokine composition of the instant disclosure.

FIG. 2A shows an illustration of a method of manufacturing a bifunctional cytokine composition by directly reacting a first cytokine and a second cytokine.

FIG. 2B shows an illustration of a method of manufacturing a bifunctional cytokine composition by reacting a first cytokine with a heterobifunctional linking reagent, then reacting the first cytokine with a second cytokine.

FIG. 2C shows an illustration of a method of manufacturing a bifunctional cytokine composition by simultaneously (e.g., in the same reaction vessel) reacting a first cytokine, a second cytokine, and a heterobifunctional linking reagent.

FIG. 2D shows an illustration of a method of manufacturing a bifunctional cytokine composition by a) reacting a first cytokine with a first heterobifunctional linking reagent, b) reacting a second cytokine with a second heterobifunctional linking reagent, and then c) reacting the first cytokine and the second cytokine.

FIG. 3A shows an illustration of an exemplary IL-2 polypeptide suitable for preparing a bifunctional cytokine composition as provided herein comprising an azide positioned at residue 45.

FIG. 3B shows an illustration of an exemplary IL-2 polypeptide suitable for preparing a bifunctional cytokine composition as provided herein comprising an azide positioned at residue 42.

FIG. 3C shows an illustration of an exemplary IL-2 polypeptide suitable for preparing a bifunctional cytokine composition as provided herein comprising an azide positioned at the N-terminus.

FIG. 4 illustrates the mechanism of action of IL-18 on IFNγ and IL-18BP production, and IL-18 inhibitory activity by IL-18BP.

FIG. 5 depicts a synthetic route which can be used to prepare a synthetic IL-18 polypeptide suitable for preparing a bifunctional cytokine composition as provided herein.

FIG. 6 illustrates a reaction of an IL-18 polypeptide with a bifunctional linking reagent (“bi-functional probe”) to prepare an IL-18 polypeptide suitable for preparing a bifunctional cytokine composition.

FIG. 7 shows SDS-PAGE analysis of bifunctional cytokine compositions provided herein comprising IL-2 and IL-18 polypeptides, as well as individual IL-2 and IL-18 polypeptides.

FIG. 8 shows dynamic light scattering (DLS) traces for the indicated bifunctional cytokine compositions and the indicated individual cytokines.

FIG. 9A shows dose response curves of IFNγ signal for the indicated IL-2/IL-18 bifunctional cytokine compositions.

FIG. 9B shows dose response curves of IFNγ release for the indicated IL-2/IL-18 bifunctional cytokine compositions.

FIG. 10 shows dose response curves for IFNγ signal in response to IL-18 polypeptide of SEQ ID NO: 30, IL-2 polypeptide of SEQ ID NO: 300, or a bifunctional cytokine composition of the same in peripheral blood mononuclear cells (PBMCs) derived from three donors.

FIG. 11A shows dose response curves for pSTAT5 in response to the indicated compositions in the indicated cell types (NK=natural killer cells; DC=dendritic cells).

FIG. 11B shows dose response curves for p65 in response to the indicated compositions in the indicated cell types (NK=natural killer cells; DC=dendritic cells).

### DETAILED DESCRIPTION OF THE INVENTION

### Bifunctional Cytokine Compositions

Provided herein are bifunctional cytokine compositions comprising a first cytokine, a second cytokine, and a linker. In some embodiments, the linker comprises a first point of attachment to the first cytokine and a second point of attachment to the second cytokine. The bifunctional cytokine compositions provide herein are effective for, in some embodiments, simultaneously delivery of both the first cytokine and the second cytokine to a target cell, such as an immune cell. In some embodiments, the simultaneous delivery of both agents to the same cell has numerous benefits, including ensuring both agents are delivered to the same cell at the same time. In some embodiments, the bifunctional cytokine compositions reduce the concentration or amount of each cytokine necessary to see a therapeutic or other benefit. In some embodiments, the first cytokine and the second cytokine have different effects on the cell, such as activating different signaling pathways. In some embodiments, the first cytokine and the second cytokine will act in a mutually beneficial way by increasing the sensitivity of the cell to the activation by the other cytokine. In some embodiments, the first cytokine and the second cytokine have synergistic effects on the cell, such as synergistic activation of the same signaling pathways. In some embodiments, the cytokines are modified relative to wild type cytokines and can have different binding properties to various cytokine receptors or receptor subunits, thereby further modulating the activity of the cytokines of the bifunctional cytokine compositions.

The conjugate compositions provided herein utilize linkers to attach the two cytokines to each other. In some embodiments, the linkers are attached to each cytokine (at specific residues or a specific subset of residues. In some embodiments, the linkers are attached to each moiety in a site-selective manner (e.g., at a pre-selected residue), such that a population of the bifunctional cytokine composition is substantially uniform. This can be accomplished in a variety of ways as provided herein, including by site-selectively adding reagents for a conjugation reaction to a moiety to be conjugated, synthesizing or otherwise preparing a moiety to be conjugated with a desired reagent for a conjugation reaction, or a combination of these two approaches. Using these approaches, the sites of attachment (such as specific amino acid residues) of the linker to each moiety can be selected with precision. Additionally, these approaches allow a variety of linkers to be employed for the composition which are not limited to amino acid residues as required for fusion proteins. For example, linkers of the instant disclosure can be chemical polymers (e.g., polyethylene glycol, poly propylene glycol, polyesters, polyamides, and combinations thereof). This combination of linker choice and precision attachment to the moieties allows the linker to also perform the function of modulating the activity of one of the moieties, for example if the linker is attached to one of the cytokines at a position that interacts with a receptor of the cytokine.

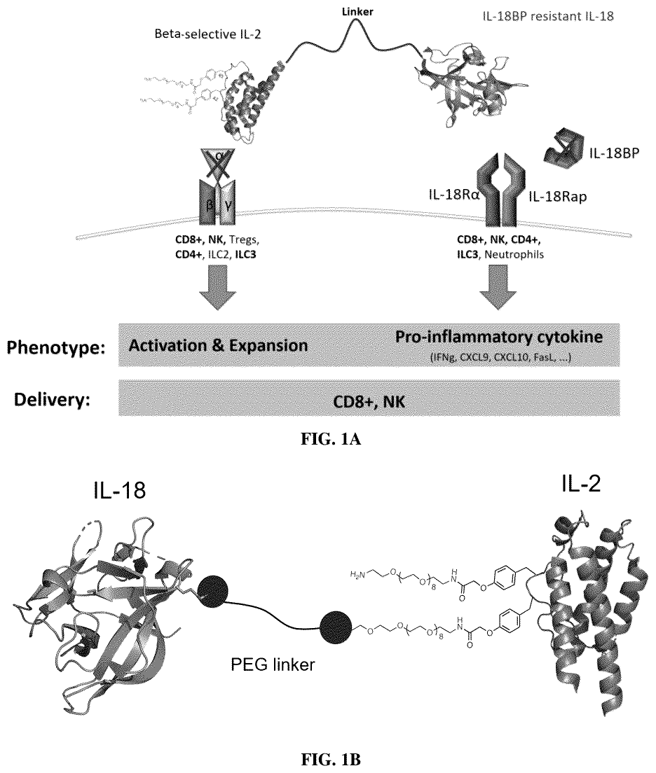

In some preferred embodiments, a bifunctional cytokine composition provided herein comprises an IL-2 polypeptide and an IL-18 polypeptide. In some embodiments, the IL-2 polypeptide does not exhibit substantial binding to or signaling through the IL-2 receptor alpha subunit. In some embodiments, the IL-18 polypeptide does not substantially bind to IL-18 binding protein. An exemplary illustration showing such a construct and proposed mechanism of action is shown in FIG. 1A. In the non-limiting, proposed mechanism, the beta-selective IL-2 signals through the IL-2 receptor βγ complex and preferentially activates and expands T_{eff} cells, such as CD8⁺, NK, and other cell types, while the IL-18 polypeptide simultaneously activates pro-inflammatory cytokines, such as IFNγ. Such constructs may be favorably delivered to CD8⁺ and NK cells owing to the receptors disposed thereon, thereby allowing for simultaneous delivery of both cytokines to stimulate a superior immune response compared to either cytokine administered alone or in a non-linked combination.

An additional exemplary, non-limiting bifunctional cytokine composition of the instant disclosure is shown in FIG. 1B. The exemplary embodiment shown is a bifunctional cytokine composition which contains an IL-2 polypeptide and an IL-18 polypeptide. The bifunctional cytokine composition shown contains a linker which comprises poly(ethylene glycol) (PEG) and is attached to residue F42Y of the IL-2 polypeptide and residue C68 of the IL-18 polypeptide. The large circles indicate the sites of reaction products of conjugation handles and complementary conjugation handles. The large circle shown on the IL-18 polypeptide indicates a reaction product of the C68 sulfhydryl group and a suitable conjugation handle on a bifunctional linking reagent (e.g., a bromoacetamide functionality or a maleimide functionality). The large circle near the center of the linker indicates a reaction product of a conjugation handle of the bifunctional linking reagent (e.g., an alkyne such as a DBCO functionality) and a conjugation handle attached to the F42Y through a PEG group (e.g., an azide functionality). The IL-2 polypeptide also comprises a PEG polymer attached to residue Y45.

### Linker Structure

In some embodiments, the linker used to attach a first cytokine (e.g., an IL-18 polypeptide as provided herein) and a second cytokine (e.g., an IL-2 polypeptide as provided herein) comprises points of attachment at both moieties. The points of attachment can be any of the residues for facilitating the attachment as provided herein. The linker structure can be any suitable structure for creating the spatial attachment between the two moieties. In some embodiments, the linker provides covalent attachment of both moieties. In some embodiments, the linker is a chemical linker (e.g., not an expressed polypeptide as in a fusion protein). In some embodiments, the linker is a peptide linker. In some embodiments, the linker is a non-peptide linker (e.g., does not consist of amino acid residues). In some embodiments, the linker is a bond between side chain functional groups of the first cytokine and the second cytokine.

### Chemical Linkers

In some embodiments, the linker is a chemical linker. In some embodiments, the chemical linker comprises at least one portion which is not comprised of amino acid residues. In some embodiments, the linker comprises a polymer. In some embodiments, the linker comprises a water soluble polymer. In some embodiments, the linker comprises poly(alkylene oxide), polysaccharide, poly(vinyl pyrrolidone), poly(vinyl alcohol), polyoxazoline, poly(acryloylmorpholine), or a combination thereof. In some embodiments, the linker comprises poly(alkylene oxide). In some embodiments, the poly(alkylene oxide) is polyethylene glycol or polypropylene glycol, or a combination thereof. In some embodiments, the poly(alkylene oxide) is polyethylene glycol.

In some embodiments, the linker comprises from 2 to 100 ethylene glycol units. In some embodiments, the linker comprises from 2 to 100, 2 to 75, 2 to 50, 2 to 40, 2 to 35, 2 to 30, 2 to 25, 5 to 100.5 to 75.5 to 50.5 to 40.5 to 35.5 to 25, 10 to 100, 10 to 75, 10 to 50, 10 to 40, 10 to 35, 10 to 30, or 10 to 25 ethylene glycol units.

In some embodiments, the linker is a bifunctional linking moiety. In some embodiments, the bifunctional linking moiety comprises an amide group, an ester group, an ether group, a thioether group, or a carbonyl group. In some embodiments, the linker comprises a non-polymer linker. In some embodiments, the linker comprises a non-polymer, bifunctional linking moiety. In some embodiments, the non-polymer, bifunctional linking moiety comprises succinimidyl 4-(N-maleimidomethyl)cyclohexane-1-carboxylate; Maleimidocaproyl; Valine-citrulline; Allyl(4-methoxyphenyl)dimethylsilane; 6-(Allyloxycarbonylamino)-1-hexanol; 4-Aminobutyraldehyde diethyl acetal; or (E)-N-(2-Aminoethyl)-4-{12-[4-(3-azidopropoxy)phenyl]diazenyl}benzamide hydrochloride.

The linker can be branched or linear. In some embodiments, the linker is linear. In some embodiments, the linker is branched. In some embodiments, the linker comprises a linear portion (e.g., between the first point of attachment and the second point of attachment) of a chain of at least 10, 20, 50, 100, 500, 1000, 2000, 3000, or 5000 atoms. In some embodiments, the linker is branched and comprises a linear portion of a chain of at least 10, 20, 50, 100, 500, 1000, 2000, 3000, or 5000 atoms. In some embodiments, the linker comprises a linear portion of at from 1 to 1000 atoms, 1 to 900 atoms, 1 to 800 atoms, 1 to 500 atoms, 1 to 400 atoms, 1 to 300 atoms, 1 to 200 atoms, 1 to 100 atoms, 1 to 50 atoms, 10 to 1000 atoms, 10 to 900 atoms, 10 to 800 atoms, 10 to 500 atoms, 10 to 400 atoms, 10 to 300 atoms, 10 to 200 atoms, 10 to 100 atoms, 10 to 50 atoms, 25 to 1000 atoms, 25 to 900 atoms, 25 to 800 atoms, 25 to 500 atoms, 25 to 400 atoms, 25 to 300 atoms, 25 to 200 atoms, 25 to 100 atoms, 25 to 50 atoms, 50 to 1000 atoms, 50 to 900 atoms, 50 to 800 atoms, 50 to 500 atoms, 50 to 400 atoms, 50 to 300 atoms, 50 to 200 atoms, or 50 to 100 atoms. In some embodiments, the linker has a linear length of from about 10 angstroms to about 200 angstroms. In some embodiments, the linker has a linear length of from about 10 to 500, 10 to 200, 10 to 150, 10 to 125, 10 to 100, 10 to 75, 10 to 50, 25 to 200, 25 to 150, 25 to 125, 25 to 100, 25 to 75, 25 to 50, 50 to 200, 50 to 150, 50 to 100, or 50 to 75 angstroms. In some embodiments, the linker has a length of up to 500 angstroms, 400 angstroms, 300 angstroms, 200 angstroms, or 100 angstroms.

In some embodiments, the linker has a molecular weight of at least about 500 Daltons, at least about 1,000 Daltons, at least about 5,000 Daltons, at least about 10,000 Daltons, at least about 15,000 Daltons, at least about 20,000 Daltons, at least about 25,000 Daltons, or at least about 30,000 Daltons. In some embodiments, the linker as a molecular weight of at most about 100,000 Daltons, at most about 50,000 Daltons, at most about 40,000 Daltons, at most about 30,000 Daltons, at most about 25,000 Daltons, at most about 20,000 Daltons at most about 15,000 Daltons, at most about 10,000 Daltons, or at most about 5,000 Daltons. In some embodiments, the linker has a molecular weight of at most about 5 kDa, 6 kDa, 7 kDa, 8 kDa, 9 kDa, 10 kDa, 15 kDa, or 20 kDa.

In some embodiments, the linker comprises a reaction product of one or more pairs of conjugation handles and a complementary conjugation handle thereof. In some embodiments, the reaction product comprises a triazole, a hydrazone, pyridazine, a sulfide, a disulfide, an amide, an ester, an ether, an oxime, an alkene, or any combination thereof. In some embodiments, the reaction product comprises a triazole. The reaction product can be separated from the first point of attachment and the second point of attachment by any portion of the linker. In some embodiments, the reaction product is substantially in the center of the linker. In some embodiments, the reaction product is substantially closer to one point of attachment than the other is.

In some embodiments, the linker comprises a structure of Formula (X)

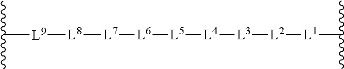

wherein each of L¹, L², L³, L⁴, L⁵, L⁶, L⁷, L⁸, and L⁹ is independently —O—, —NR^{L}—, —(C₁-C₆ alkylene)NR^{L}—, —NR^{L}(C₁-C₆ alkylene)-, —N(R^{L})₂⁺—, —(C₁-C₆ alkylene)N(R^{L})₂⁺—, —N(R^{L})₂⁺—(C₁-C₆ alkylene)-, —OP(═O)(OR^{L})O—, —S—, —(C₁-C₆ alkylene)S—, —S(C₁-C₆ alkylene)-, —S(═O)—, —S(═O)₂—, —C(═O)—, —(C₁-C₆ alkylene)C(═O)—, —C(═O) (C₁-C₆ alkylene)-, —C(═O)O—, —OC(═O)—, —OC(═O)O—, —C(═O)NR^{L}—, —C(═O)NR^{L}(C₁-C₆ alkylene)-, —(C₁-C₆ alkylene)C(═O)NR^{L}—, —NR^{L}C(═O)—, —(C₁-C₆ alkylene)NR^{L}C(═O)—, —NR^{L}C(═O)(C₁-C₆ alkylene)-, —OC(═O)NR^{L}—, —NR^{L}C(═O)O—, —NR^{L}C(═O)NR^{L}—, —NR^{L}C(═S)NR^{L}—, —CR^{L}═N—, —N═CR^{L}, —NR^{L}S(═O)₂—, —S(═O)₂NR^{L}—, —C(═O)NR^{L}S(═O)₂—, —S(═O)₂NR^{L}C(═O)—, substituted or unsubstituted C₁-C₆ alkylene, substituted or unsubstituted C₁-C₆ heteroalkylene, substituted or unsubstituted C₂-C₆ alkenylene, substituted or unsubstituted C₂-C₆ alkynylene, substituted or unsubstituted C₆-C₂₀ arylene, substituted or unsubstituted C₂-C₂₀ heteroarylene, —(CH₂—CH₂—O)_{qa}—, —(O—CH₂—CH₂)_{qb}—, —(CH₂—CH(CH₃)—O)_{qc}—, —(O—CH(CH₃)—CH₂)_{qd}—, a reaction product of a conjugation handle and a complementary conjugation handle, or absent; (C₁-C₆ alkylene)
each R^{L} is independently hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₈ cycloalkyl, substituted or unsubstituted C₂-C₇ heterocycloalkyl, substituted or unsubstituted aryl, or substituted or unsubstituted heteroaryl; and
each of qa, qb, qc and qd is independently an integer from 1-100,
wherein each

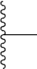

is a point of attachment to the first cytokine or the second cytokine.

In some embodiments, the linker consists of a plurality of structures of Formula (X) to form the linkage between the first cytokine and the second cytokine (e.g., 1, 2, 3, 4, 5, 6, 7, 8, 9, 10, or more structures of Formula (X) appended from end to end, where only the terminal

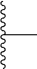

denote points of attachment to the first cytokine or the second cytokine).

In some embodiments, the linker comprises a structure of Formula (X′)

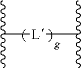

wherein each L′ is independently —O—, —NR^{L}—, —(C₁-C₆ alkylene)NR^{L}—, —NR^{L}(C₁-C₆ alkylene)-, —N(R^{L})₂⁺—, —(C₁-C₆ alkylene)N(R^{L})₂⁺—, —N(R^{L})₂⁺—(C₁-C₆ alkylene)-, —OP(═O)(OR^{L})O—, —S—, —C₆ alkylene)S—, —S(C₁-C₆ alkylene)-, —S(═O)—, —S(═O)₂—, —C(═O)—, —(C₁-C₆ alkylene)C(═O)—, —C(═O) (C₁-C₆ alkylene)-, —C(═O)O—, —OC(═O)—, —OC(═O)O—, —C(═O)NR^{L}—, —C(═O)NR^{L}(C₁-C₆ alkylene)-, —(C₁-C₆ alkylene)C(═O)NR^{L}—, —NR^{L}C(═O)—, —(C₁-C₆ alkylene)NR^{L}C(═O)—, —NR^{L}C(═O)(C₁-C₆ alkylene)-, —OC(═O)NR^{L}—, —NR^{L}C(═O)O—, —NR^{L}C(═O)NR^{L}—, —NR^{L}C(═S)NR^{L}—, —CR^{L}═N—, —N═CR^{L}, —NR^{L}S(═O)₂—, —S(═O)₂NR^{L}—, —C(═O)NR^{L}S(═O)₂—, —S(═O)₂NR^{L}C(═O)—, substituted or unsubstituted C₁-C₆ alkylene, substituted or unsubstituted C₁-C₆ heteroalkylene, substituted or unsubstituted C₂-C₆ alkenylene, substituted or unsubstituted C₂-C₆ alkynylene, substituted or unsubstituted C₆-C₂₀ arylene, substituted or unsubstituted C₂-C₂₀ heteroarylene, —(CH₂—CH₂—O)_{qa}—, —(O—CH₂—CH₂)_{qb}—, —(CH₂—CH(CH₃)—O)_{qc}—, —(O—CH(CH₃)—CH₂)_{qd}—, a reaction product of a conjugation handle and a complementary conjugation handle, or absent; (C₁-C₆ alkylene);
each R^{L} is independently hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₈ cycloalkyl, substituted or unsubstituted C₂-C₇ heterocycloalkyl, substituted or unsubstituted aryl, or substituted or unsubstituted heteroaryl; and
each of qa, qb, qc and qd is independently an integer from 1-100,
g is an integer from 1-100,
wherein each

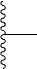

is a point of attachment to the first cytokine (e.g., IL-18) or the second cytokine (e.g., IL-2).

In some embodiments, each reaction product of a conjugation handle and a complementary conjugation handle independently comprises a triazole, a hydrazone, pyridazine, a sulfide, a disulfide, an amide, an ester, an ether, an oxime, or an alkene. In some embodiments, each reaction product of a conjugation handle and a complementary conjugation handle comprises a triazole. In some embodiments, each reaction product of a conjugation handle and a complementary conjugation handle comprise a structure of

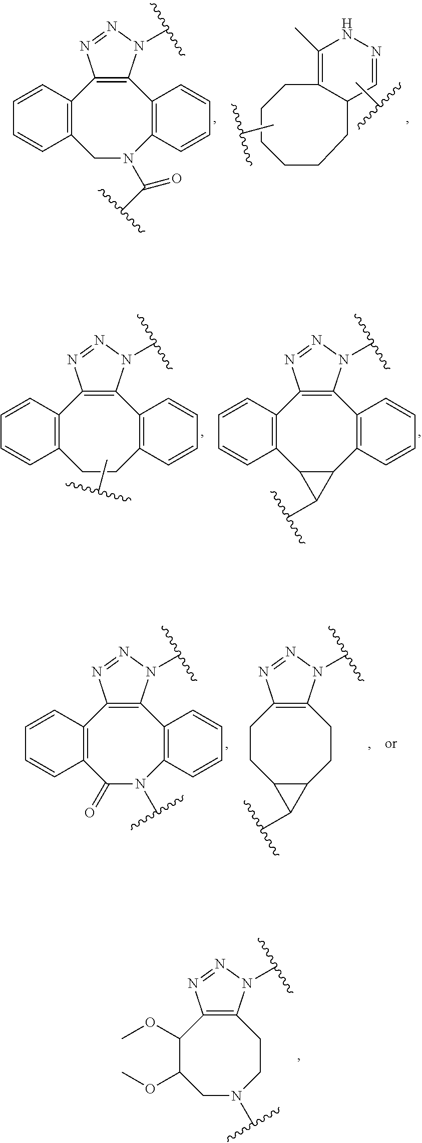

or a regioisomer or derivative thereof. In some embodiments, the linker comprises a single reaction product of a conjugation handle and a complementary conjugation handle. In some embodiments, the linker comprises 2 reaction products of a conjugation handle and a complementary conjugation handle, wherein each reaction product is different. In some embodiments, the linker comprises 3 reaction products of a conjugation handle and a complementary conjugation handle, wherein each reaction product, wherein no more than 2 of the reaction products are the same.

In some embodiments, at least a portion of the linker is formed from a reaction of one or both cytokines with a bifunctional linking reagent. Exemplary bifunctional linking reagents useful for this purpose are of a formula A-B-C, wherein A is a first conjugation handle reactive with a complementary conjugation handle on the first cytokine (e.g., maleimide, α,β-unsaturated carbonyl, a-halogenated carbonyl for a first cytokine wherein a cysteine is the conjugation handle attached thereto), B is a linking group, and C is a second conjugation handle reactive with a complementary conjugation handle on the second cytokine (e.g., an alkyne such as DBCO for a second cytokine wherein an azide is the conjugation handle attached thereto). Specific non-limiting examples of bifunctional linking reagents include

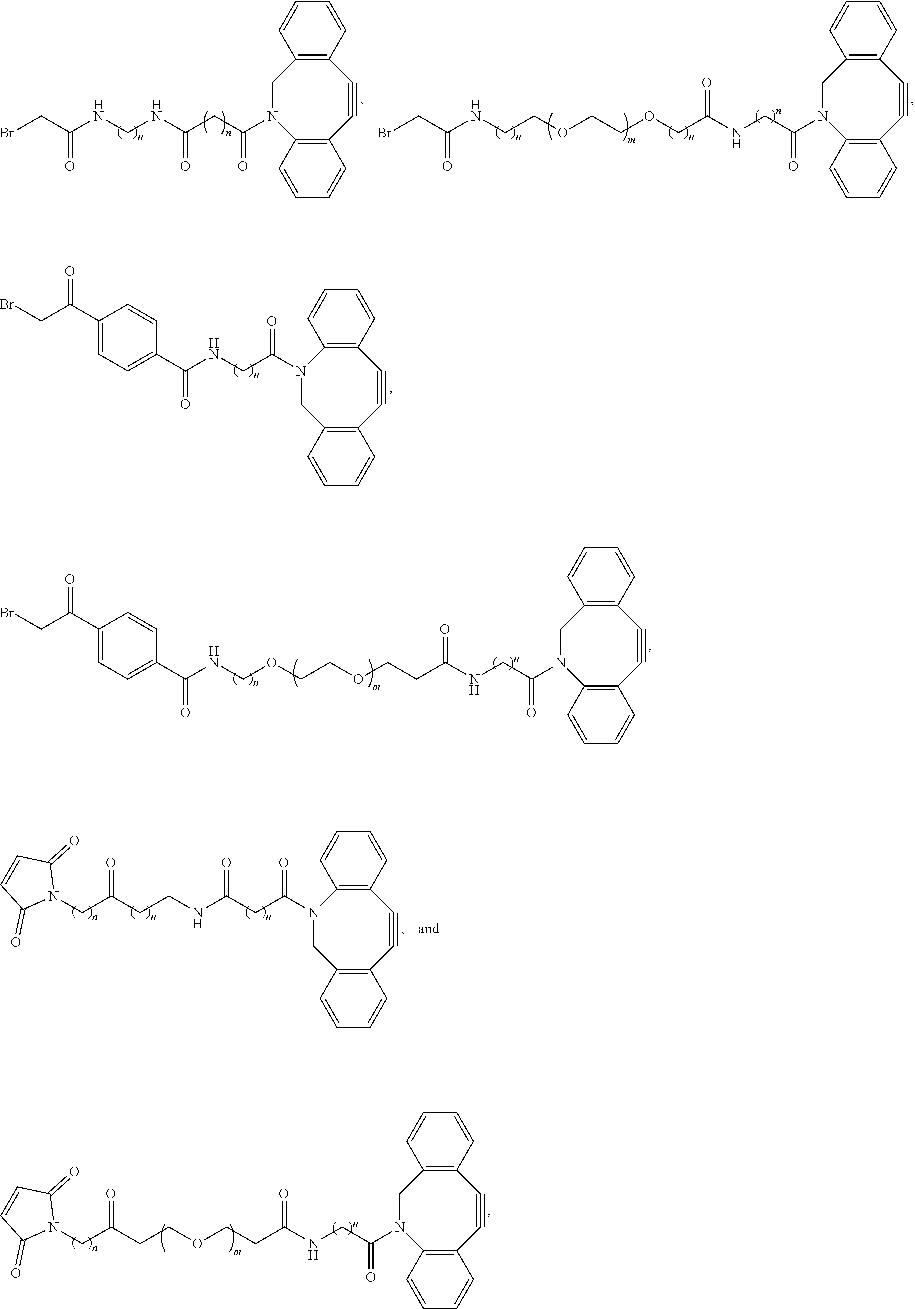

wherein each n is independently an integer from 1-6 and each m is independently an integer from 1-30, and related molecules (e.g., isomers, sulfate modified versions, and the like).

In some embodiments, the linker comprises a structure formed from a bifunctional linking reagent. In some embodiments, the linker comprises a structure:

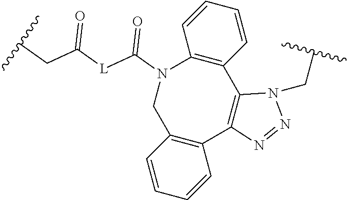

wherein

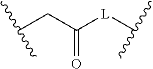

is a point of attachment to a cysteine of a first cytokine (e.g., an IL-18 polypeptide); L is a linking group; and

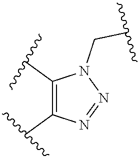 [SMILES: *Cn1nnc(*)c1*]

is a point of attachment to a second cytokine (e.g., an IL-2 polypeptide) formed from a reaction of an azide attached to the second cytokine,
or a regioisomer thereof. The point of attachment to the second cytokine depicted in the formula can be through a suitable linkage group. It is also expressly contemplated that the reaction products of conjugation handles depicted in the above formula can be replaced with alternative reaction products of conjugation handles.

In some embodiments, L has a structure

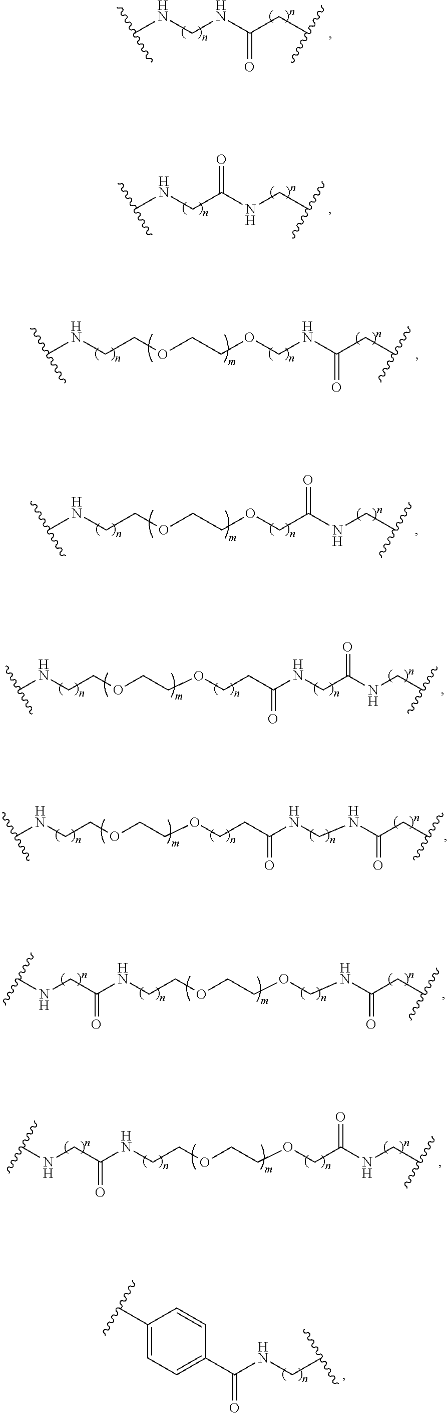

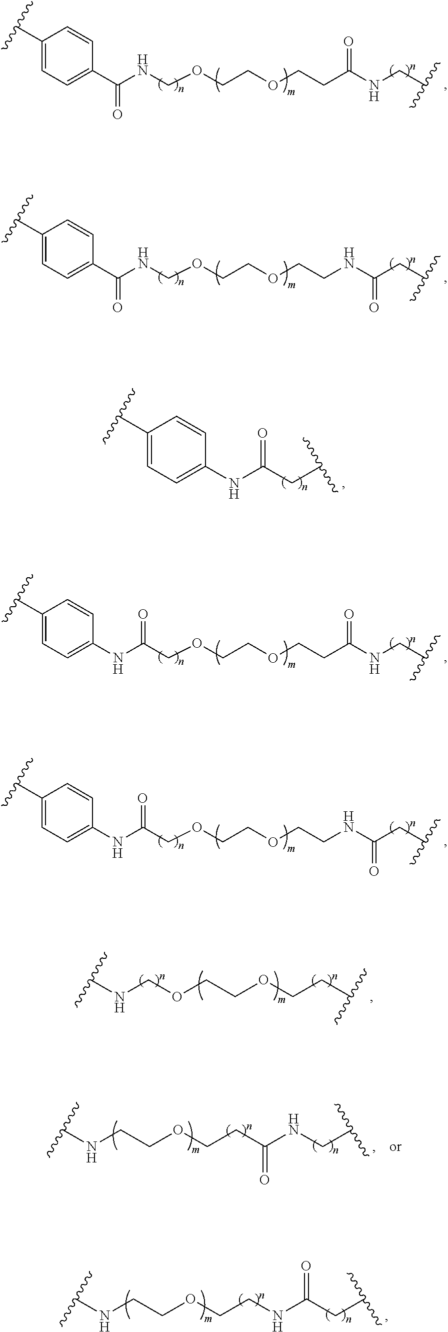

wherein each n is independently an integer from 1-6 and each m is an integer from 1-30, or a derivatized version thereof (e.g., sulfate modified). In some embodiments, each m is independently 2 or 3. In some embodiments, each in is an integer from 1-24, from 1-18, from 1-12, or from 1-6.

In some embodiments, the linker comprises a structure:

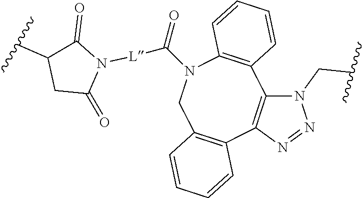

wherein

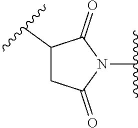 [SMILES: *C1CC(=O)N(*)C1=O]

is a point of attachment to a cysteine of a first cytokine (e.g., an IL-18 polypeptide); L″ is a linking group; and

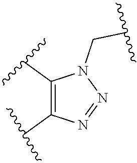 [SMILES: *Cn1nnc(*)c1*]

is a point of attachment to a second cytokine (e.g., an IL-2 polypeptide) formed from a reaction of an azide attached to the second cytokine,
or a regioisomer thereof, or a structure wherein one of the bonds of the succinimide is hydrolyzed. The point of attachment to the second cytokine depicted in the formula can be through a suitable linkage group. It is also expressly contemplated that the reaction products of conjugation handles depicted in the above formula can be replaced with alternative reaction products of conjugation handles.

In some embodiments, L″ has a structure

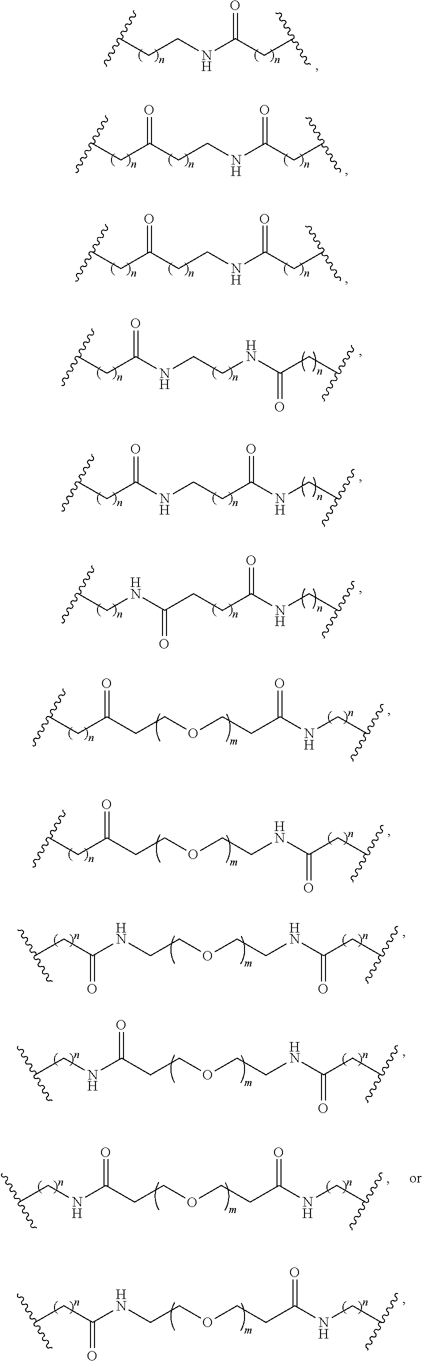

wherein each n is independently an integer from 1-6 and each m is independently an integer from 1-30, or a derivatized version thereof (e.g., sulfate modified). In some embodiments, each m is independently 2 or 3. In some embodiments, each m is an integer from 1-24, from 1-18, from 1-12, or from 1-6.

### Peptide Linkers

In some embodiments, the first cytokine and the second cytokine are linked through a peptide linker. In some embodiments, the first cytokine is linked to the second cytokine as a fusion protein. In such instances, the linker comprises one or more peptide bonds between the first cytokine and the second cytokine. In some embodiments, the linker of the fusion protein between the first cytokine and the second cytokine is a bond. In some embodiments, the linker of the fusion protein between the first cytokine and the second cytokine is a linking peptide. Non-limiting examples of linking peptides include, but are not limited to (GS). (SEQ ID NO: 200), (GGS)ₙ(SEQ ID NO: 201), (GGGS)ₙ (SEQ ID NO: 202), (GGSG)ₙ (SEQ ID NO: 203), or (GGSGG)ₙ (SEQ ID NO: 204), (GGGGS)ₙ (SEQ ID NO: 205), wherein n is 1, 2, 3, 4, 5, 6, 7, 8, 9, or 10. For example, a linking peptide can be (GGGGS)₃ (SEQ ID NO: 206) or (GGGGS)₄ (SEQ ID NO: 207). In some embodiments, the first cytokine is fused to the C-terminal end of the second cytokine (optionally through a linking peptide). In some embodiments, the first cytokine is fused to the N-terminal end of the second cytokine (optionally through a linking peptide).

### Cleavable Linkers

In some embodiments, the linker (e.g., a chemical or peptide linker as provided herein) is a cleavable linker. In some embodiments, the cleavable linker is cleaved at, near, or in a tumor microenvironment. In some embodiments, the tumor is mechanically or physically cleaved at, near, or in the tumor microenvironment. In some embodiments, the tumor is chemically cleaved at, near, or in a tumor microenvironment. In some embodiments, the cleavable linker is a reduction sensitive linker. In some embodiments, the cleavable linker is an oxidation sensitive linker. In some embodiments, the cleavable linker is cleaved as a result of pH at, near, or in the tumor microenvironment. In some embodiments, the cleavable linker is cleaved by a tumor metabolite at, near, or in the tumor microenvironment. In some embodiments, the cleavable linker is cleaved by a protease at, near, or in the tumor microenvironment.

### Conjugation Handle Chemistry

In some embodiments, the first cytokine, the second cytokine, or both will comprise a conjugation handle which is used to conjugate the first cytokine to the second cytokine, or with a bifunctional linking reagent which is used to from the linker between the first cytokine and the second cytokine.

Any suitable reactive group capable of reacting with a complementary reactive group attached to the synthetic cytokine or derivative thereof can be used as the conjugation handle. In some embodiments, the conjugation handle comprises a reagent for a Cu(I)-catalyzed or “copper-free” alkyne-azide triazole-forming reaction (e.g., strain promoted cycloadditions), the Staudinger ligation, inverse-electron-demand Diels-Alder (IEDDA) reaction, “photo-click” chemistry, tetrazine cycloadditions with trans-cycloctenes, potassium acyl trifluoroborate (KAT) ligation, or a metal-mediated process such as olefin metathesis and Suzuki-Miyaura or Sonogashira cross-coupling.

In some embodiments, the conjugation handle comprises a reagent for a “copper-free” alkyne azide triazole-forming reaction. Non-limiting examples of alkynes for said alkyne azide triazole forming reaction include cyclooctyne reagents (e.g., (1R,8S,9s)-Bicyclo[6.1.0]non-4-yn-9-ylmethanol containing reagents, dibenzocyclooctyne-amine reagents, difluorocyclooctynes, or derivatives thereof). In some embodiments, the alkyne functional group is attached to the Fc region. In some embodiments, the azide functional group is attached to the Fc region.

In some embodiments, the conjugation handle comprises a reactive group selected from azide, alkyne, tetrazine, halide, sulfhydryl, disulfide, maleimide, activated ester, alkene, aldehyde, ketone, imine, hydrazine, and hydrazide. In some embodiments, the synthetic cytokine or derivative thereof comprises a reactive group complementary to the conjugation handle of the Fc region. In some embodiments, the conjugation handle and the complementary conjugation handle comprise “CLICK” chemistry reagents. Exemplary groups of click chemistry residue are shown in Hein et al., “Click Chemistry, A Powerful Tool for Pharmaceutical Sciences,” Pharmaceutical Research, volume 25, pages 2216-2230 (2008); Thirumurugan et al., “Click Chemistry for Drug Development and Diverse Chemical-Biology Applications,” Chem. Rev. 2013, 113, 7, 4905-4979; US20160107999A1; U.S. Ser. No. 10/266,502B2; and US20190204330A1, each of which is incorporated by reference in its entirety.

### Cytokines and Derivatives Thereof

Cytokines are proteins produced in the body that are important in cell signaling. Cytokines can modulate the immune system, and cytokine therapy utilizes the immunomodulatory properties of the molecules to enhance or regulate the immune system of a subject. Disclosed herein are bifunctional cytokine compositions which comprise cytokines (e.g., modified cytokines and/or synthetic cytokines) linked with other cytokines using a linker as provided herein. In some embodiments, bifunctional cytokine compositions of the instant disclosure can exhibit enhanced biological activity compared to individual cytokines by themselves or can modulate the immune system in advantageous ways difficult to achieve with individual cytokines.

A cytokine of a bifunctional cytokine composition as provided herein can be any cytokine. Non-limiting examples of cytokines include interleukins (e.g., IL-2, IL-18, IL-7, IL-17), TNF family cytokines (e.g., TNFa, CD70, TNFSF14), interferons (e.g., IFNγ, IFNα. IFNβ), TGF-β family cytokines (e.g., TGFB1, TGFB2, TGFB3), chemokines (e.g., CCL2, CCL3, CXCL9, CXCL10) and others. In some embodiments, one or both of the cytokines of the bifunctional cytokine composition is an interleukin. In some embodiments, each interleukin is independently selected from an IL-1 family cytokine (e.g., IL-18, IL-1β, IL-33), an IL-2 family cytokine (e.g., IL-2, IL-4, IL-7, IL-15, IL-21), an IL-6 family interleukin (e.g., IL-6, IL-11, IL-31), an IL-10 family cytokine (e.g., IL-10, IL-19, IL-20, IL-22), an IL-12 family cytokine (e.g., IL-12, IL-23, IL-27, IL-35) and an IL-17 family cytokine (e.g., IL-17, IL-17F, IL-25). In some embodiments, each cytokine of the bifunctional cytokine composition is a different interleukin. In some embodiments, each cytokine of the bifunctional cytokine composition is independently selected from an IL-2 polypeptide, an IL-7 polypeptide, an IL-12 polypeptide, and an IL-18 polypeptide.

Cytokines of the bifunctional cytokine compositions provided herein may be modified versions of the cytokines. In some embodiments, the cytokines comprise modifications (e.g., amino acid substitutions, additions, or deletions, attachment of polymers) which can modulate the activity of the cytokine (e.g., enhance activity, detune activity, or modulate the activity, such as by biasing the cytokine to one receptor or receptor subunit). In some embodiments, the cytokines comprise modifications in order to facilitate site specific attachment of a linker as provided herein. Additionally, cytokines provided herein may also be fused to additional polypeptides (e.g., antibody fusions, Fc fusions, etc.) or peptide sequences, such as artificial leader sequences, half-life extension sequences, or other peptides affixed to the N or C terminus of the cytokine. Cytokines may also be truncated versions of cytokines provided herein.

A cytokine as provided herein may be of a size which is amenable to linking to an additional cytokine in a bifunctional cytokine composition. In some embodiments, a cytokine as provided herein is of a size which is amenable to chemical synthesis. In some embodiments, at least one cytokine of the bifunctional cytokine composition comprises from about 50 to about 300 amino acid residues, from about 50 to about 250 amino acid residues, from about 50 to about 200 amino acid residues, from about 75 to about 300 amino acid residues, from about 75 to about 250 amino acid residues, from about 75 to about 200 amino acid residues, from about 100 to about 300 amino acid residues, from about 100 to about 250 amino acid residues, or from about 100 to about 200 amino acid residues. In some embodiments, each cytokine of the bifunctional cytokine composition independently comprise from about 50 to about 300 amino acid residues, from about 50 to about 250 amino acid residues, from about 50 to about 200 amino acid residues, from about 75 to about 300 amino acid residues, from about 75 to about 250 amino acid residues, from about 75 to about 200 amino acid residues, from about 100 to about 300 amino acid residues, from about 100 to about 250 amino acid residues, or from about 100 to about 200 amino acid residues.

A cytokine of a bifunctional cytokine composition provided herein can be synthetic or recombinant. In some embodiments, both cytokines of the bifunctional cytokine composition are synthetic. In some embodiments, both cytokines of the bifunctional cytokine composition are recombinant. In some embodiments, one cytokine of the bifunctional cytokine composition is synthetic and the other cytokine is recombinant.

The cytokines of the bifunctional cytokine composition each comprise a point of attachment to the linker. The linker can be attached to any residue of a cytokine of the bifunctional cytokine composition. In some embodiments, the linker is attached to at least one cytokine at a position that is not the N-terminal amine or the C-terminal carboxyl of the cytokine. In some embodiments, the linker is attached to at least one cytokine at a non-terminal residue of the cytokine. In some embodiments, the linker is attached to at least one cytokine at a residue which is not the N-terminal residue or the C-terminal residue. In some embodiments, the linker is attached to at least one cytokine at an internal residue of the cytokine. In some embodiments, the linker is attached to each cytokine at a position which is not the N-terminal amine or C-terminal carboxyl of each cytokine.

In some embodiments, the point of attachment to the linker is a pre-selected residue of one or both cytokines. In some embodiments, the pre-selected residue is one for which the linker can be attached specifically (e.g., can be attached without substantial attachment to other residues in a reaction). In some embodiments, the pre-selected residue comprises a conjugation handle for attachment of the linker.

### Interleukin 18 (IL-18) Cytokines and Derivatives Thereof

In some embodiments, at least one cytokine of a bifunctional cytokine composition provided herein is an IL-18 polypeptide. After stimulation with antigen plus IL-12, naïve T cells develop into IL-18 receptor (IL-18R)-expressing Th1 cells, which increase IFN-γ production in response to IL-18 stimulation. IL-18 is a proinflammatory cytokine that facilitates type 1 responses. IL-18 without IL-12, but with IL-2, stimulates NK cells, CD4⁺ NKT cells, and established Th1 cells, to produce IL-3, IL-9, and IL-13. Concomitant with IL-3, IL-18 stimulates mast cells and basophils to produce IL-4, IL-13, and chemical mediators (e.g., histamine). IL-18 is a member of the IL-1 family of cytokines. Murine and human IL-18 proteins consist of 192 and 193 amino acids, respectively. IL-18 is produced as a biologically inactive precursor, pro-IL-18, which lacks a signal peptide and requires proteolytic processing to become active. The cleavage of pro-IL-18 or pro-IL-1β depends mainly on the action of the intracellular cysteine protease caspase-1 in the NLRP3 inflammasome. The IL-18 receptor (IL-18R) consists of the inducible component IL-18Rα (IL-1 receptor-related protein [IL-1Rrp]) and the constitutively expressed component IL-18Rβ (IL-1R accessory protein-like [IL-1RAcPL]). Cytoplasmic domains of IL-18Rα and IL-18Rβ contain a common domain termed the Toll-like receptor (TLR)/IL-1R (TIR) domain. Upon stimulation with IL-18, IL-18Rα forms a high-affinity heterodimeric complex with IL-18Rβ, which mediates intracellular signal transduction. Cytoplasmic TIR domains of the receptor complex interact with myeloid differentiation primary response 88 (MyD88), a signal adaptor containing a TIR domain, via TIR-TIR interactions. Then, MyD88-induced events result in the activation of nuclear factor (NF)-κB and mitogen-activated protein kinase (MAPK) via association with the signal adaptors IL-1R-associated kinase (IRAK) 1-4 and tumor necrosis factor (TNF) receptor-activated factor (TRAF) 6, respectively, which eventually leads to the appropriate gene expressions, such as Ifng, Tnfa, Cd40l, and FasL.

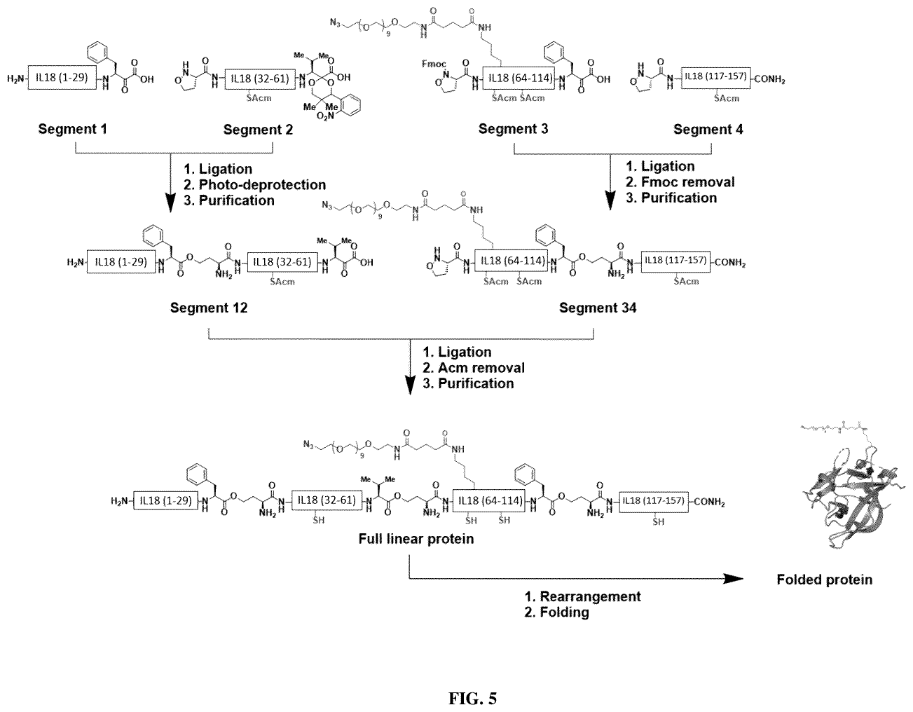

In vivo, the activity of IL-18 is balanced by the presence of a high affinity, naturally occurring IL-18 binding protein (IL-18BP). IL-18BP binds IL-18 and neutralizes the biological activity of IL-18. Cell surface IL-18Rα competes with IL-18BP for IL-18 binding. Increased disease severity can be associated with an imbalance of IL-18 to IL-18BP such that levels of free IL-18 are elevated in the circulation. FIG. 4 illustrates the mechanism of action of IL-18, IFNγ production, IL-18BP production, and inhibition of IL-18 activity by IL-18BP. IL-18 induces IFNγ production, which in turn induces IL-18BP production. IL-18BP then competes with IL-18Rα to inhibit IL-18 activity. This feedback loop of IL-18BP production after stimulation of IFNγ production has limited the effectiveness of IL-18 as a treatment modality in previous efforts. Thus, in some embodiments, an IL-18 polypeptide of a bifunctional cytokine composition provided herein comprises one or more modifications which result in the bifunctional cytokine composition having a lower affinity for IL-18BP.

Non-limiting examples of IL-18 amino acid sequences to be utilized in embodiments described herein are provided below in SEQ ID NOs: 1-72. An IL-18 polypeptide utilized in conjugate (e.g., a bifunctional cytokine composition) described herein can have, for example, an amino acid sequence that is at least 80%, 85%, 90%, 91%, 92%, 93%, 94%, 95%, 96%, 97%, 98%, 99%, or 100% identical to any one of the sequences in Table 4. In some embodiments, an IL-18 polypeptide utilized in a bifunctional cytokine composition can be any of those IL-18 polypeptides described in Table 4′.

### Linker Points of Attachment to IL-18 Polypeptides

Bifunctional cytokine compositions comprising an IL-18 polypeptide provided herein comprise linkers as discussed supra, including chemical linkers. When an IL-18 polypeptide is used in a bifunctional cytokine composition, the point of attachment to the IL-18 polypeptide is to a residue as provided herein.

In some embodiments, the linker is attached to an amino acid residue of the IL-18 polypeptide. In some embodiments, the linker is attached to any amino acid residue of the IL-18 polypeptide (e.g., at a position corresponding to any one of positions 1-157 of SEQ ID NO: 1). In some embodiments, the linker is attached at a non-terminal residue (e.g., a residue other than the C-terminal residue or N-terminal residue) of the IL-18 polypeptide (e.g., a residue at position corresponding to any one of positions 2-156 of SEQ ID NO: 1). In some embodiments, the linker is attached at a non-terminal residue of the IL-18 polypeptide, wherein the IL-18 polypeptide has been extended or truncated by one or more amino acids relative to SEQ ID NO: 1.

In some embodiments, the linker is attached to the IL-18 polypeptide at a residue in a region comprising residues 2-156, wherein residue position numbering is based on SEQ ID NO: 1 as a reference sequence. In some embodiments, the linker is attached to the IL-18 polypeptide at a residue in ta region comprising residues 30-150. In some embodiments, the linker is attached to the IL-18 polypeptide at a residue in a region comprising residues 33-43, residues 60-100, residues 65-75, residues 80-90, residues 85-100, residues 90-110, residues 115-130, residues 120-130, or residues 140-150. In some embodiments, the linker is attached to the IL-18 polypeptide at a residue selected from residue 38, 68, 69, 70, 76, 78, 85, 86, 95, 98, 121, 127, and 144. In some embodiments, the linker is attached to the IL-18 polypeptide at a residue selected from 68, 69, 70, 85, 86, and 98. In some embodiments, the linker is attached to the IL-18 polypeptide at residue 68, 69, or 70. In some embodiments, the linker is attached to the IL-18 polypeptide at residue 85, 86, 95, or 98. In some embodiments, the linker is attached to the IL-18 polypeptide at residue 68. In some embodiments, the linker is attached to the IL-18 polypeptide at residue 69. In some embodiments, the linker is attached to the IL-18 polypeptide at residue 70. In some embodiments, the linker is attached to the IL-18 polypeptide at residue 85. In some embodiments, the linker is attached to the IL-18 polypeptide at residue 86. In some embodiments, the linker is attached to the IL-18 polypeptide at residue 95. In some embodiments, the linker is attached to the IL-18 polypeptide at residue 98.

In some embodiments, the linker is attached to the IL-18 polypeptide at a residue which is known in the art to be compatible with attachment of a polymer to the IL-18 polypeptide without having a profound impact on the bioactivity of the IL-18 polypeptide. Examples of these residues include residues 38, 76, 78, 121, 127, and 144, as described in PCT Pub. No. WO2004091517A2, which is hereby incorporated by reference as if set forth in its entirety.

In some embodiments, the residue to which the linker is attached is a natural amino acid residue. In some embodiments, the residue to which the linker is covalently attached is selected from cysteine, aspartate, asparagine, glutamate, glutamine, serine, threonine, lysine, and tyrosine. In some embodiments, the residue to which the linker is covalently attached is selected from asparagine, aspartic acid, cysteine, glutamic acid, glutamine, lysine, and tyrosine. In some embodiments, the linker is covalently attached to a cysteine. In some embodiments, the linker is covalently attached to a lysine. In some embodiments, the linker is covalently attached to a glutamine. In some embodiments, the linker is covalently attached to an asparagine. In some embodiments, the residue to which the linker is attached is a tyrosine. In some embodiments, the residue to which the linker is attached is the natural amino acid in that position in SEQ ID NO: 1.

In some embodiments, the linker is attached to a different natural amino acid which is substituted at the relevant position. The substitution can be for a naturally occurring amino acid which is more amenable to attachment of additional functional groups (e.g., aspartic acid, cysteine, glutamic acid, lysine, serine, threonine, or tyrosine), a derivative of modified version of any naturally occurring amino acid, or any unnatural amino acid (e.g., an amino acid containing a desired reactive group, such as a CLICK chemistry reagent such as an azide, alkyne, etc.). In some embodiments, the linker is covalently attached to site-specifically to a natural amino acid.

In some embodiments, the linker is attached at an unnatural amino acid residue. In some embodiments, the unnatural amino acid residue comprises a conjugation handle. In some embodiments, the conjugation handle facilitates the addition of the linker to the modified IL-18 polypeptide. The conjugation handle can be any of the conjugation handles provided herein. In some embodiments, the linker is covalently attached site-specifically to the unnatural amino acid. Non-limiting examples of amino acid residues comprising conjugation handles can be found, for example, in PCT Pub. Nos. WO2015054658A1, WO2014036492A1, and WO2021133839A1 WO2006069246A2, and WO2007079130A2, each of which is incorporated by reference as if set forth in its entirety.

In some embodiments, the linker is covalently attached at residue 68. In some embodiments, the linker is covalently attached at residue C68, C68E, C68D, C68Q, C68K, C68N, or C68Y. In some embodiments, the linker is covalently attached at residue C68. In some embodiments, the linker is covalently attached to an unnatural amino acid at residue 68.

In some embodiments, the linker is covalently attached at residue 69. In some embodiments, the linker is covalently attached at residue E69, E69C, E69D, E69Q, E69K, E69N, or E69Y. In some embodiments, the linker is covalently attached at residue E69. In some embodiments, the linker is covalently attached residue E69C. In some embodiments, the linker is covalently attached to an unnatural amino acid at residue 69.

In some embodiments, the linker is covalently attached at residue 70. In some embodiments, the linker is covalently attached at residue K70, K70C, K70D, K70Q, K70E, K70N, or K70Y. In some embodiments, the linker is covalently attached at residue K70. In some embodiments, the linker is covalently attached residue K70C. In some embodiments, the linker is covalently attached to an unnatural amino acid at residue 70.

In some embodiments, the linker is covalently attached at residue 85. In some embodiments, the linker is covalently attached at residue E85, E85C, E85D, E85Q, E85K, E85N, or E85Y. In some embodiments, the linker is covalently attached at residue E85. In some embodiments, the linker is covalently attached residue E85C. In some embodiments, the linker is covalently attached to an unnatural amino acid at residue 85.

In some embodiments, the linker is covalently attached at residue 86. In some embodiments, the linker is covalently attached at residue M86C, M86D, M86Q, M86K, M86N, M86E, or M86Y. In some embodiments, the linker is covalently attached M86C. In some embodiments, the linker is covalently attached to an unnatural amino acid at residue 86.

In some embodiments, the linker is covalently attached at residue 95. In some embodiments, the linker is covalently attached at residue T95, T95C, T95D, T95Q, T95K, T95N, T95E, or T95Y. In some embodiments, the linker is covalently attached at residue T95C, T95D, T95Q, T95K, T95N, T95E, or T95Y. In some embodiments, the linker is covalently attached at residue T95C. In some embodiments, the linker is covalently attached to an unnatural amino acid at residue 95.

In some embodiments, the linker is covalently attached at residue 98. In some embodiments, the linker is covalently attached at residue D98, D98C, D98Q, D98K, D98N, D98E, or D98Y. In some embodiments, the linker is covalently attached at residue D98C. In some embodiments, the linker is covalently attached to an unnatural amino acid at residue 98.

In some embodiments, the linker is covalently attached through a modified natural amino acid. In some embodiments, the modified natural amino acid comprises a conjugation handle. In some embodiments, the linker is covalently attached through a modified amino acid α. In some embodiments, the modified amino acid α is an amino-acid-PEG-azide group or an amino-acid-PEG-alkyne group. In some embodiments, the modified amino acid α is a glutamate, aspartate, lysine, cysteine, or tyrosine modified to incorporate an azide, alkyne, or other conjugation handle group linked to the amino acid through a PEG spacer. In some embodiments, the modified amino acid α has a structure selected from:

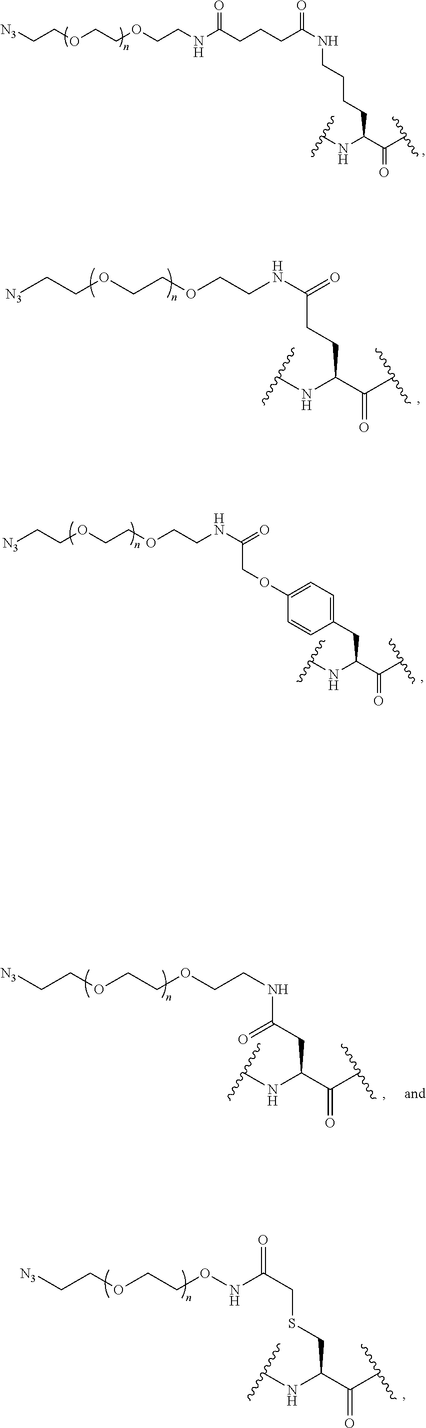

wherein each n is independently an integer from 1-30. In some embodiments, the modified amino acid α has a structure selected from:

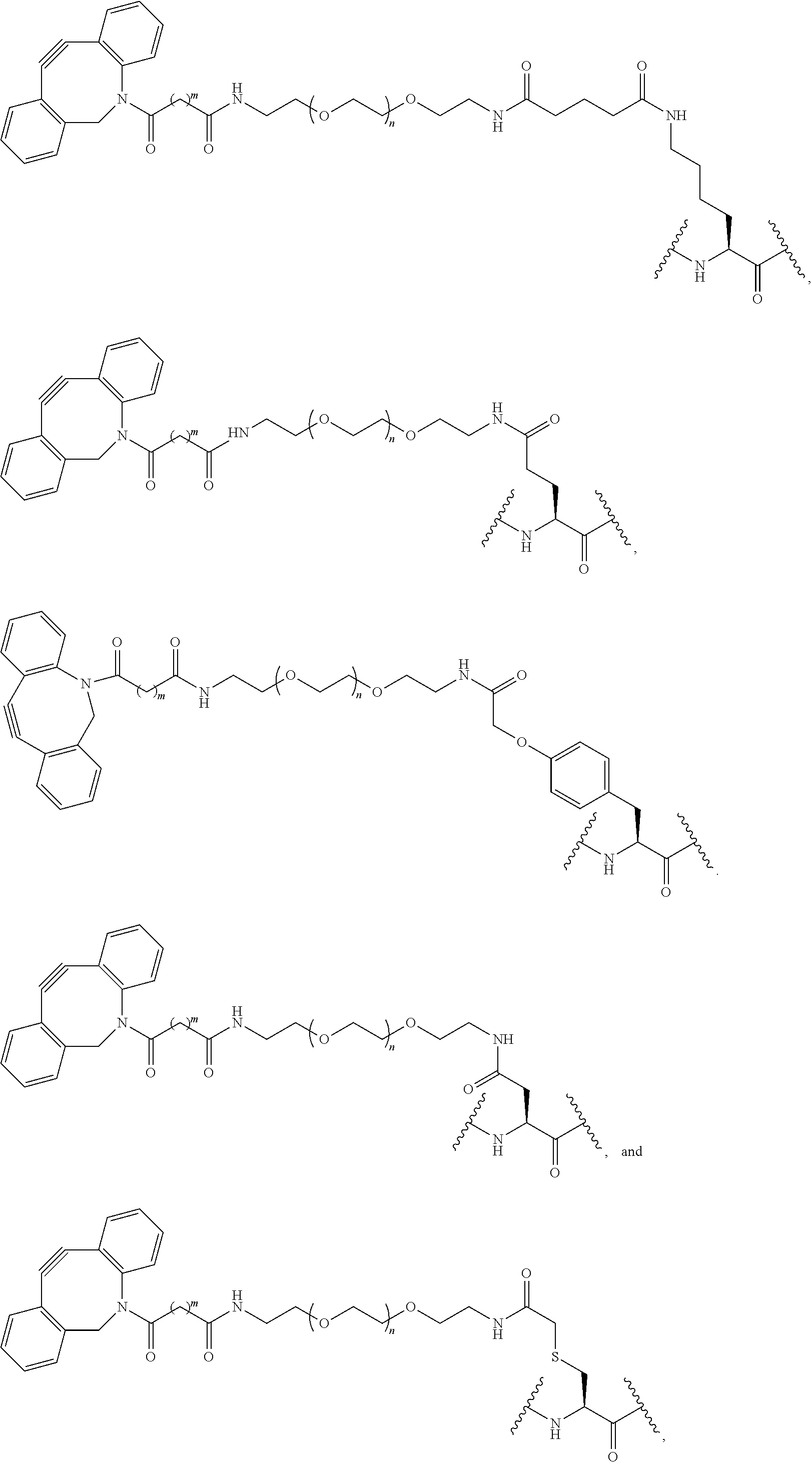

wherein each n is independently an integer from 1-30 and each n is independently an integer from 2-10. In some embodiments, n is an integer from 1-20, 1-10, 2-30, 2-20, 2-10, 5-30, 5-20, or 5-10. In some embodiments, n is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In some embodiments, n is 10. In some embodiments, n is 8. In some embodiments, n is 6. In some embodiments, n is 12. In some embodiments, each m is independently 2, 3, 4, 5, 6, 7, 8, 9, or 10. In some embodiments, m is 3. In some embodiments, m is 4. In some embodiments, m is 5. In some embodiments, m is 6. The modified amino acid α can be incorporated at any point of attachment of the IL-18 polypeptide as provided herein. In some embodiments, the modified amino acid α is located at a position on the modified IL-18 polypeptide selected from residue 68, residue 69, residue 70, residue 85, residue 86, residue 95, or residue 98.

Where IL-18 polypeptides contain unnatural amino acids or modified natural amino acids (e.g., those provided herein for purposes of conjugation), these amino acids may be incorporated into the IL-18 polypeptides using many techniques known in the art for introduction such modifications. For example, recombinant proteins with unnatural amino acids can be made using methods as described in Patent Cooperation Treaty Publication Nos. WO2016115168, WO2002085923, WO2005019415, and WO2005003294. Alternatively or in combination, unnatural or modified natural amino acids can be incorporated into chemically synthesized proteins during synthesis.

### Modifications to IL-18 Polypeptides

In some embodiments, the IL-18 polypeptide of the bifunctional cytokine composition comprises one or more modifications to that of SEQ ID NO: 1. The modifications provided herein are in addition to any modification at the point of attachment as discussed supra. In some embodiments, the residue position numbering of the modified IL-18 polypeptide is based on SEQ ID NO: 1 as a reference sequence.

Modifications to the IL-18 polypeptide described herein encompass mutations, addition of various functionalities, deletion of amino acids, addition of amino acids, or any other alteration of the wild-type version of the protein or protein fragment. Functionalities which may be added to polypeptides include polymers, linkers, alkyl groups, detectable molecules such as chromophores or fluorophores, reactive functional groups, or any combination thereof. In some embodiments, functionalities are added to individual amino acids of the polypeptides. In some embodiments, functionalities are added site-specifically to the polypeptides.

In some embodiments, the IL-18 polypeptide of the bifunctional cytokine composition comprise one or more modifications in addition to a modification needed to attach the linker to the relevant residue of the IL-18 polypeptide (e.g., an amino acid substitution at a residue to which the linker is not attached). In some embodiments, the modification is in the range of amino acid residues 1-127, based on the sequence of human IL-18³⁷⁻¹⁹³ (SEQ ID NO: 1). SEQ ID NO: 1 reflects the bioactive form of IL-18. Endogenously, IL-18 is initially expressed with an additional 36 amino acid segment at the N-terminus which is cleaved by caspases to mediate biologic activity.

In some embodiments, the IL-18 polypeptide of the bifunctional cytokine composition described herein contain 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more modified amino acid residues.

In some embodiments, the IL-18 polypeptide of the bifunctional cytokine composition comprises an amino acid sequence having at least 80%, 85%, 90%, 91%, 92%, 93%, 94%, 95%, 96%, 97%, 98%, or 99% sequence identity to the sequence set forth in SEQ ID NO: 1.

In some embodiments, the IL-18 polypeptide of the bifunctional cytokine composition provided herein comprises an amino acid sequence of any one of SEQ ID NOs: 2-67 provided herein. In some embodiments, the IL-18 polypeptide comprises an amino acid sequence at least 85% identical to the sequence of any one of SEQ ID NOs: 2-67. In some embodiments, the IL-18 polypeptide of the bifunctional cytokine composition provided herein comprises an amino acid sequence of any one of SEQ ID NOs: 68-72 provided herein. In some embodiments, the IL-18 polypeptide comprises an amino acid sequence at least 85% identical to the sequence of any one of SEQ ID NOs 68-72. In some embodiments, the IL-18 polypeptide comprises an amino acid sequence of SEQ ID NO: 30. In some embodiments, the IL-18 polypeptide comprises an amino acid sequence at least 85% identical to the sequence of SEQ ID NO: 30. In some embodiments, the IL-18 polypeptide comprises an amino acid sequence of SEQ ID NO: 59. In some embodiments, the IL-18 polypeptide comprises an amino acid sequence at least 85% identical to the sequence of SEQ ID NO: 59. In some embodiments, the IL-18 polypeptide of the bifunctional cytokine composition comprises the amino acid sequence set forth in SEQ ID NO: 68. In some embodiments, the IL-18 polypeptide of the bifunctional cytokine composition comprises the amino acid sequence set forth in SEQ ID NO: 69. In some embodiments, the IL-18 polypeptide of the bifunctional cytokine composition comprises the amino acid sequence set forth in SEQ ID NO: 70. In some embodiments, the IL-18 polypeptide of the bifunctional cytokine composition comprises the amino acid sequence set forth in SEQ ID NO: 71. In some embodiments, the IL-18 polypeptide of the bifunctional cytokine composition comprises the amino acid sequence set forth in SEQ ID NO: 72. In some embodiments, the IL-18 polypeptide is one of those described in Table 4′.

In some embodiments, the IL-18 polypeptide of the bifunctional cytokine composition described herein comprises at least 1, at least 2, at least 3, at least 4, at least 5, at least 6, at least 7, or at least 9 amino acid substitutions, wherein the amino acid substitutions are relative to SEQ ID NO: 1. In some embodiments, the IL-18 polypeptide comprises 1 to 9 amino acid substitutions. In some embodiments, the IL-18 polypeptide comprises 1 or 2 amino acid substitutions, 1 to 3 amino acid substitutions, 1 to 4 amino acid substitutions, 1 to 5 amino acid substitutions, 1 to 6 amino acid substitutions, 1 to 7 amino acid substitutions, 1 to 8 amino acid substitutions, 2 to 3 amino acid substitutions, 2 to 4 amino acid substitutions, 2 to 5 amino acid substitutions, 2 to 6 amino acid substitutions, 2 to 7 amino acid substitutions, 2 to 8 amino acid substitutions, 2 to 9 amino acid substitutions 3 or 4 amino acid substitutions, 3 to 5 amino acid substitutions, 3 to 6 amino acid substitutions, 3 to 7 amino acid substitutions, 3 to 9 amino acid substitutions, 4 or 5 amino acid substitutions, 4 to 6 amino acid substitutions, 4 to 7 amino acid substitutions, 4 to 9 amino acid substitutions, 5 or 6 amino acid substitutions, 5 to 7 amino acid substitutions, 5 to 9 amino acid substitutions, 6 or 7 amino acid substitutions, 6 to 9 amino acid substitutions, or 7 to 9 amino acid substitutions. In some embodiments, the IL-18 polypeptide comprises 3 amino acid substitutions, 4 amino acid substitutions, 5 amino acid substitutions, 6 amino acid substitutions, 7 amino acid substitutions, or 9 amino acid substitutions. In some embodiments, the IL-18 polypeptide comprises at most 4 amino acid substitutions, 5 amino acid substitutions, 6 amino acid substitutions, 7 amino acid substitutions, or 9 amino acid substitutions.

In some embodiments, the IL-18 polypeptide comprising of the bifunctional cytokine composition described herein comprises at least 1, at least 2, at least 3, at least 4, at least 5, at least 6, at least 7, or at least 9 natural amino acid substitutions, wherein the natural amino acid substitutions are relative to SEQ ID NO: 1. In some embodiments, the IL-18 polypeptide comprises 1 to 9 natural amino acid substitutions. In some embodiments, the IL-18 polypeptide comprises 1 or 2 natural amino acid substitutions, 1 to 3 natural amino acid substitutions, 1 to 4 natural amino acid substitutions, 1 to 5 natural amino acid substitutions, 1 to 6 natural amino acid substitutions, 1 to 7 natural amino acid substitutions, 1 to 8 natural amino acid substitutions, 2 to 3 natural amino acid substitutions, 2 to 4 natural amino acid substitutions, 2 to 5 natural amino acid substitutions, 2 to 6 natural amino acid substitutions, 2 to 7 natural amino acid substitutions, 2 to 8 natural amino acid substitutions, 2 to 9 natural amino acid substitutions, 3 or 4 natural amino acid substitutions, 3 to 5 natural amino acid substitutions, 3 to 6 natural amino acid substitutions, 3 to 7 natural amino acid substitutions, 3 to 9 natural amino acid substitutions, 4 or 5 natural amino acid substitutions, 4 to 6 natural amino acid substitutions, 4 to 7 amino acid substitutions, 4 to 9 natural amino acid substitutions, 5 or 6 natural amino acid substitutions, 5 to 7 amino acid substitutions, 5 to 9 natural amino acid substitutions, 6 or 7 natural amino acid substitutions, 6 to 9 natural amino acid substitutions, or 7 to 9 natural amino acid substitutions. In some embodiments, the IL-18 polypeptide comprises 3 natural amino acid substitutions, 4 natural amino acid substitutions, 5 amino acid substitutions, 6 natural amino acid substitutions, 7 natural amino acid substitutions, or 9 natural amino acid substitutions. In some embodiments, the IL-18 polypeptide comprises at most 4 natural amino acid substitutions, 5 natural amino acid substitutions, 6 natural amino acid substitutions, 7 natural amino acid substitutions, or 9 natural amino acid substitutions. In some embodiments, the IL-18 polypeptide further comprises up to 10 non-canonical amino acid substitutions. In some embodiments, the IL-18 polypeptide comprises 1, 2, 3, 4, 5, 6, 7, 8, 9 or 10 unnatural amino acid substitutions. In some embodiments, the IL-18 polypeptide further comprises unnatural amino acid substitutions at residues M33, M51, N60, M86, M113, and/or M150. In some embodiments, the unnatural amino acid residues substituted for the methionines are each independently norleucine or O-methyl-homoserine. In some embodiments, the IL-18 polypeptide further unnatural amino acid substitutions at residues 31, 75, and 116. In some embodiments, the IL-18 polypeptide further comprises homoserine (Hse) 31, Hse 75, and Hse 116. In some embodiments, the IL-18 polypeptide further unnatural amino acid substitutions at residues 31, 63, and 116. In some embodiments, the IL-18 polypeptide further comprises homoserine (Hse) 31, Hse 63, and Hse 116. In some embodiments, the modified IL-18 polypeptide further unnatural amino acid substitutions at residues 31, 63, 75, and 116. In some embodiments, the modified IL-18 polypeptide further comprises homoserine (Hse) 31, Hse 63, Hse 75, and Hse 116. In some embodiments, the modified IL-18 polypeptide further unnatural amino acid substitutions at residues 31, 67, 75, and 116. In some embodiments, the modified IL-18 polypeptide further comprises homoserine (Hse) 31, Hse 67, Hse 75, and Hse 116. In some embodiments, the modified IL-18 polypeptide further unnatural amino acid substitutions at residues 31, 57, 75, and 116. In some embodiments, the modified IL-18 polypeptide further comprises homoserine (Hse) 31, Hse 57, Hse 75, and Hse 116. In some embodiments, the modified IL-18 polypeptide further unnatural amino acid substitutions at residues 31, 50, 75, and 116. In some embodiments, the modified IL-18 polypeptide further comprises homoserine (Hse) 31, Hse 50, Hse 75, and Hse 116. In some embodiments, the modified IL-18 polypeptide further unnatural amino acid substitutions at residues 31, 50, 75, and 121. In some embodiments, the modified IL-18 polypeptide further comprises homoserine (Hse) 31, Hse 50, Hse 75, and Hse 121.

In some embodiments, the IL-18 polypeptide of the bifunctional cytokine composition described herein comprises at least 1, at least 2, at least 3, at least 4, at least 5, at least 6, at least 7, or at least 9 amino acid substitutions, wherein the amino acid substitutions are relative to any one of SEQ ID NOs: 68, 92, 116, 140, or 170. In some embodiments, a modified IL-18 polypeptide comprising a polymer described herein comprises at least 1, at least 2, at least 3, at least 4, at least 5, at least 6, at least 7, or at least 9 additional amino acid substitutions, wherein the amino acid substitutions are relative to any one of SEQ ID NOs: 68, 92, 116, 140, or 170. In some embodiments, the IL-18 polypeptide comprises 1 to 9 amino acid substitutions. In some embodiments, the IL-18 polypeptide comprises 1 or 2 amino acid substitutions, 1 to 3 amino acid substitutions, 1 to 4 amino acid substitutions, 1 to 5 amino acid substitutions, 1 to 6 amino acid substitutions, 1 to 7 amino acid substitutions, 1 to 8 amino acid substitutions, 2 to 3 amino acid substitutions, 2 to 4 amino acid substitutions, 2 to 5 amino acid substitutions, 2 to 6 amino acid substitutions, 2 to 7 amino acid substitutions, 2 to 8 amino acid substitutions, 2 to 9 amino acid substitutions 3 or 4 amino acid substitutions, 3 to 5 amino acid substitutions, 3 to 6 amino acid substitutions, 3 to 7 amino acid substitutions, 3 to 9 amino acid substitutions, 4 or 5 amino acid substitutions, 4 to 6 amino acid substitutions, 4 to 7 amino acid substitutions, 4 to 9 amino acid substitutions, 5 or 6 amino acid substitutions, 5 to 7 amino acid substitutions, 5 to 9 amino acid substitutions, 6 or 7 amino acid substitutions, 6 to 9 amino acid substitutions, or 7 to 9 amino acid substitutions. In some embodiments, the IL-18 polypeptide comprises 3 amino acid substitutions, 4 amino acid substitutions, 5 amino acid substitutions, 6 amino acid substitutions, 7 amino acid substitutions, or 9 amino acid substitutions. In some embodiments, the IL-18 polypeptide comprises at most 4 amino acid substitutions, 5 amino acid substitutions, 6 amino acid substitutions, 7 amino acid substitutions, or 9 amino acid substitutions.

In some embodiments, one modification is at amino acid residue 6. In some embodiments, one modification is in the range of amino acid residues 53-63. In some embodiments, one modification is at amino acid residue 53. In some embodiments, one modification is at amino acid residue 63.

In some embodiments, the IL-18 polypeptide comprises at least one modification to the amino acid sequence of SEQ ID NO: 1 selected from: Y01X, F02X, E06X, S10X, V11X, D17X, C38X, M51X, K53X, D54X, S55X, T63X, C68X, C76X, AND C127X, wherein each X is independently a natural or non-natural amino acid. In some embodiments, the IL-18 polypeptide further comprises an amino acid substitution at the point of attachment of the linker, such as residue 69, residue 70, residue 85, residue 86, residue 95, or residue 98. In some embodiments, the IL-18 polypeptide comprises at least one modification to the amino acid sequence of SEQ ID NO: 1 selected from: Y01G, F02A, E06K, S10T, V11I, D17N, C38S, C38A, C38Q, M51G, K53A, D54A, S55A, T63A, C68S, C68A, C76S, C76A, C127A, and C127S. In some embodiments, the IL-18 polypeptide further comprises an amino acid substitution at the point of attachment of the linker, such as E69C, K70C, E85C, M86C, T95C, or D98C.

In one aspect, described herein is a modified interleukin-18 (IL-18) polypeptide with a polymer as provided herein, comprising a modified IL-18 polypeptide comprising E06K and K53A, wherein residue position numbering of the IL-18 polypeptide is based on SEQ ID NO: 1 as a reference sequence. In some embodiments, the IL-18 polypeptide further comprises V11I. In some embodiments, the IL-18 polypeptide further comprises T63A. In some embodiments, the IL-18 polypeptide further comprises at least one of Y01X, S55X, F02X, D54X, C38X, C68X, E69X, K70X, C76X, or C127X, wherein each X is independently an amino acid or an amino acid derivative. In some embodiments, the IL-18 polypeptide further comprises at least one of Y01G, S55A, F02A, D54A, C38S, C38A, C38Q, C68S, C68A, K70C, C76S, C76A, C127S, or C127A. In some embodiments, the IL-18 polypeptide further comprises an amino acid substitution at the point of attachment of the linker, such as residue 69, residue 70, residue 85, residue 86, residue 95, or residue 98. In some embodiments, the IL-18 polypeptide further comprises a V11I substitution.

In some embodiments, the IL-18 peptide comprises at least one modification to the amino acid sequence of SEQ ID NO: 1, wherein the modification is E06X, V11X, K53X, S55X, or T63X, wherein X is a natural or non-natural amino acid. In some embodiments, the IL-18 peptide comprises at least two modifications to the amino acid sequence of SEQ ID NO: 1, wherein the modifications comprise E06X and K53X; E06X and S55X; K53X and S55X; E06X and T63X; or K53X and T63X, wherein X is a natural or non-natural amino acid. In some embodiments, the IL-18 peptide comprises at least three modifications to the amino acid sequence of SEQ ID NO: 1, wherein the modifications comprise E06X, K53X, and S55X; or E06X, K53X, and T63X, wherein X is a natural or non-natural amino acid. In some embodiments, the IL-18 peptide comprises at least four modifications to the amino acid sequence of SEQ ID NO: 1, wherein the modifications comprise E06X, K53X, S55X, and T63X; E06X, K53X, S55X, and Y01X; E06X, K53X, S55X, and F02X; E06X, K53X, S55X, and D54X; E06X, K53X, S55X, and M51X; or C38X, C68X, C76X, and C127X, wherein X is a natural or non-natural amino acid. In each embodiment wherein a plurality of amino acids residues are replaced with a natural or non-natural amino acid X, each X is independently the same or a different amino acid.

In some embodiments, the IL-18 peptide comprises at least one modification to the amino acid sequence of SEQ ID NO: 1, wherein the modification is E06K, V11I, K53A, S55A, or T63A. In some embodiments, the IL-18 peptide comprises at least two modifications to the amino acid sequence of SEQ ID NO: 1, wherein the modifications comprise E06K and K53A; E06K and S55A; K53A and S55A; E06K and T63A; or K53A and T63A. In some embodiments, the IL-18 peptide comprises at least three modifications to the amino acid sequence of SEQ ID NO: 1, wherein the modifications comprise E06K, K53A, and S55A; E06K, V11I, and K53A; E06K, C38A, and K53A; or E06K, K53A, and T63A. In some embodiments, the IL-18 peptide comprises at least four modifications to the amino acid sequence of SEQ ID NO: 1, wherein the modifications comprise E06K, K53A, S55A, and T63A; E06K, K53A, S55A, and Y01G; E06K, K53A, S55A, and F02A; E06K, K53A, S55A, and D54A; E06K, K53A, S55A, and M51G; or C38S, C68S, C76S, and C127S. In some embodiments, the IL-18 peptide comprises at least six modifications to the amino acid sequence of SEQ ID NO: 1, wherein the modifications comprise E06K, K53A, C38S, C68S, C76S, and C127S; or K53A, T63A, C38S, C68S, C76S, and C127S. In some embodiments, the modified IL-18 polypeptide comprises at least seven modifications to the sequence of SEQ ID NO: 1, wherein the seven modifications comprise E6K, V11I, C38A, K53A, T63A, C76A, C127A. In some embodiments, the IL-18 peptide comprises at least eight modifications to the amino acid sequence of SEQ ID NO: 1, wherein the modifications comprise Y01G, F02A, E06K, M51G, K53A, D54A, S55A, and T63A. In some embodiments, the IL-18 peptide comprises at least eight modifications to the amino acid sequence of SEQ ID NO: 1, wherein the modifications comprise Y01G, F02A, E06K, M51G, K53A, D54A, S55A, and T63A.

In one aspect, provided herein, is a modified IL-18 polypeptide with a polymer as provided herein (e.g., a polymer attached to a residue as provided herein), further comprising E06K and K53A, wherein residue position numbering of the IL-18 polypeptide is based on SEQ ID NO: 1 as a reference sequence. In some embodiments, the IL-18 polypeptide comprises an amino acid sequence at least 80%, at least 85%, at least 90%, at least 95%, or at least 98% identical to the amino acid sequence of SEQ ID NO: 30. In some embodiments, the IL-18 polypeptide comprises an amino acid sequence at least 80%, at least 85%, at least 90%, at least 95%, or at least 98% identical to the amino acid sequence of SEQ ID NO: 59. In some embodiments, the IL-18 polypeptide further comprises an amino acid substitution at one or more cysteine residues. In some embodiments, the IL-18 polypeptide comprises one or more cysteines substituted with either serine or alanine. In some embodiments, the IL-18 polypeptide comprise amino acid substitutions at each cysteine residue of SEQ ID NO: 1. In some embodiments, each cysteine residue is substituted with serine or alanine. In some embodiments, the IL-18 polypeptide comprises amino acid substitutions at 1, 2, 3, 4, 5, or 6 methionine residues. In some embodiments, each substitution at a methionine residue is for an O-methyl-L-homoserine residue or a norleucine residue. In some embodiments, each methionine residue is substituted with an O-methyl-L-homoserine residue. In some embodiments, the IL-18 polypeptide comprises homoserine residues at positions 31, 116, and one of 63 and 75. In some embodiments, the modified IL-18 polypeptide comprises homoserine residues at positions 31, 116, 75, and one of 50, 57, 63, and 67. In some embodiments, the modified IL-18 polypeptide comprises homoserine residues at positions 31, 121, 75, and one of 50, 57, 63, and 67.

In some embodiments, the IL-18 polypeptide comprises a polypeptide sequence having at least about 80%, at least about 85%, at least about 90%, at least about 95%, at least about 98%, at least about 99%, or about 100% sequence identity to SEQ ID NO: 2-12. In some embodiments, the IL-18 polypeptide comprises a polypeptide sequence having at least about 80%, at least about 85%, at least about 90%, at least about 95%, at least about 98%, at least about 99%, or about 100% sequence identity to SEQ ID NO: 13-23. In some embodiments, the modified IL-18 polypeptide comprises a polypeptide sequence having at least about 80%, at least about 85%, at least about 90%, at least about 95%, at least about 98%, or at least about 99% sequence identity to SEQ ID NO: 24-33 In some embodiments, the polypeptide sequence is at least about 80% identical to SEQ ID NO: 30 or SEQ ID NO: 59. In some embodiments, the polypeptide sequence is at least about 80% identical to SEQ ID NO: 30. In some embodiments, the polypeptide sequence is at least about 90% identical to SEQ ID NO: 30. In some embodiments, the polypeptide sequence is at least about 95% identical to SEQ ID NO: 30. In some embodiments, the polypeptide sequence is at least about 80% identical to SEQ ID NO: 59. In some embodiments, the polypeptide sequence is at least about 90% identical to SEQ ID NO: 59. In some embodiments, the polypeptide sequence is at least about 95% identical to SEQ ID NO: 59. In some embodiments, the IL-18 polypeptide is recombinant. In some embodiments, the IL-18 polypeptide is synthetic. An exemplary synthetic route for preparing a synthetic IL-18 polypeptide is depicted in FIG. 5.

### Biological Activity of IL-18 Polypeptides

In some embodiments, the bifunctional cytokine composition exhibits one or more activities associated with the IL-18 polypeptide of the bifunctional cytokine composition.

In some embodiments, the bifunctional cytokine composition exhibits an ability to bind to the IL-18 receptor. In some embodiments, the bifunctional cytokine composition exhibits an ability to bind to the IL-18 receptor (IL-18Rαβ) which is comparable to WT IL-18. In some embodiments, bifunctional cytokine composition exhibits an ability to bind to the IL-18 receptor which is reduced by at most 2-fold, at most 5-fold, at most 10-fold, at most 20-fold, at most 50-fold, at most 100-fold, at most 200-fold, at most 300-fold, at most 400-fold, or at most 1000-fold compared to WT IL-18. In some embodiments, the bifunctional cytokine composition exhibits an enhanced ability to bind the IL-18 receptor. In some embodiments, the bifunctional cytokine composition exhibits an ability to bind to the IL-18Rαβ which is increased by at least 2-fold, at least 3-fold, at least 5-fold, or at least 10-fold compared to WT IL-18.

In some embodiments, bifunctional cytokine composition exhibits an ability to stimulate production of IFNγ upon contact with a cell (e.g., an immune cell, such as an NK cell). In some embodiments, the ability of the bifunctional cytokine composition to stimulate IFNγ production is somewhat reduced compared to WT IL-18. In some embodiments, a half-maximal effective concentration (EC₅₀) of the ability of the bifunctional cytokine composition to stimulate production of IFNγ is at most 100-fold higher than, at most 50-fold higher than, at most 20-fold higher than, at most 10-fold higher than, at most 5-fold higher than, or at most 2-fold higher than that of a WT IL-18.

In some embodiments, the bifunctional cytokine composition exhibits a reduced ability to bind IL-18 binding protein (IL-18BP). In some embodiments, the ability of bifunctional cytokine composition to bind IL-18BP is reduced by at least 2-fold, at least 4-fold, at least 5-fold, at least 10-fold, at least 20-fold, at least 30-fold, at least 50-fold, or at least 100-fold compared to WT IL-18. In some embodiments, the bifunctional cytokine composition does not display any substantial ability to bind IL-18 BP.

In some embodiments, the bifunctional cytokine composition exhibits a reduced ability to have its IFNγ production stimulatory activity inhibited by IL-18BP. In some embodiments, the ability of the bifunctional cytokine composition to be inhibited by IL-18BP is measured as a half maximal inhibitory concentration (IC₅₀). In some embodiments, the bifunctional cytokine composition exhibits an IC₅₀ by IL-18BP that is at least 2-fold higher than, at least 4-fold higher than, at least 8-fold higher than, at least 10-fold higher than, at least 15-fold higher than, at least 20-fold higher than, at least 30-fold higher than, at least 50-fold higher than, or at least 100-fold higher than an IC₅₀ of WT IL-18's inhibition by IL-18BP.

### Interleukin-2 (IL-2) Cytokines and Derivatives Thereof

In some embodiments, at least one cytokine of a bifunctional cytokine composition provided herein is an IL-2 polypeptide. Interleukin-2 (IL-2) is a cytokine signaling molecule important in regulating the immune system. IL-2 is implicated in helping the immune system differentiate between foreign and endogenous cell types, thereby preventing the immune system from attacking a subject's own cells. IL-2 accomplishes its activity through interactions with IL-2 receptors (IL-2R) expressed by lymphocytes. Through these binding interactions, IL-2 can modulate a subject's populations of T-effector (T_{eff}) cells, natural killer (NK) cells, and regulatory T-cells (T_{reg}).

IL-2 has been used to treat cancer, both alone and in combination with other therapies. However, use of IL-2 as a treatment has been limited by the toxicity of IL-2, undesirable side effects such as vascular leak syndrome, and the short half-life of IL-2. Conjugation of IL-2 to a second cytokine of the disclosure can improve IL-2 polypeptide selectivity, enhance the therapeutic potential of IL-2, and minimize the risk of side effects from administering IL-2 therapies. The present disclosure describes antibodies or antigen binding fragments conjugated to a modified and/or synthetic interleukin-2 (IL-2) polypeptide and the use of the conjugates as therapeutic agents. Modified IL-2 polypeptides provided herein can be used as immunotherapies or as parts of other immunotherapy regimens. Such modified IL-2 polypeptides may display binding characteristics for the IL-2 receptor (IL-2R) that differ from wild-type IL-2. In one aspect, modified IL-2 polypeptides described herein have decreased affinity for the IL-2R αβγ complex (IL-2Rα). In some embodiments, the modified IL-2 polypeptides have an increased affinity for the IL-2R βγ complex (IL-2Rβ). In some embodiments, the binding affinity between the modified IL-2 polypeptides and IL-2Rβ is the same as or lower than the binding affinity between a wild-type IL-2 and IL-2Rβ. Non-limiting examples of IL-2 amino acid sequences to be utilized in embodiments described herein are provided below in Table 4A. An IL-2 polypeptide utilized in conjugate described herein can have, for example, an amino acid sequence that is at least 80%, 85%, 90%, 91%, 92%, 93%, 94%, 95%, 96%, 97%, 98%, 99%, or 100% identical to any one of the sequences in Table 4A.

### Linker Points of Attachment to IL-2 Polypeptides

Bifunctional cytokine compositions comprising an IL-2 polypeptide provided herein comprise linkers as discussed supra, including chemical linkers. When an IL-2 polypeptide is used in a bifunctional cytokine composition, the point of attachment to the IL-2 polypeptide is to a residue as provided herein.

In some embodiments, the linker is attached to an amino acid residue of the IL-2 polypeptide. In some embodiments, the linker is attached to any amino acid residue of the IL-2 polypeptide (e.g., at a position corresponding to any one of positions 1-133 of SEQ ID NO: 301). In some embodiments, the linker is attached at a non-terminal residue (e.g., a residue other than the C-terminal residue or N-terminal residue) of the IL-2 polypeptide (e.g., a residue at position corresponding to any one of positions 2-132 of SEQ ID NO: 301). In some embodiments, the linker is attached at a non-terminal residue of the IL-18 polypeptide, wherein the IL-2 polypeptide has been extended or truncated by one or more amino acids relative to SEQ ID NO: 301 (e.g., the linker is attached to a residue corresponding to residue 2 of SEQ ID NO: 301 and residue 1 of SEQ ID NO: 301 has been deleted). In some embodiments, the linker is attached to the N-terminal residue of the IL-2 polypeptide. In some embodiments, the linker is attached to the N-terminal amine of the IL-2 polypeptide. In some embodiments, the linker is attached to the C-terminal residue of the IL-2 polypeptide. In some embodiments, the linker is attached to the C-terminal carboxyl group of the IL-2 polypeptide.

In some embodiments, the linker is attached to the IL-2 polypeptide at a residue in a region comprising residues 2-132, wherein residue position numbering is based on SEQ ID NO: 301 as a reference sequence. In some embodiments, the linker is attached to the IL-2 polypeptide at a residue in ta region comprising residues 30-75. In some embodiments, the linker is attached to the IL-2 polypeptide at a residue in a region comprising residues 35-55, residues 35-50, residues 35-45, residues 30-50, residues 40-45, residues 60-75, residues 60-70, residues 65-70, or residues 2-5. In some embodiments, the linker is attached to the IL-2 polypeptide at a residue selected from residue 65, 66, 67, 68, 69, and 70. In some embodiments, the linker is attached to the IL-2 polypeptide at a residue selected from residue 40, 41, 42, 43, 44, and 45. In some embodiments, the linker is attached to the IL-2 polypeptide at residue 42 or 45. In some embodiments, the linker is attached to the IL-2 polypeptide at residue 42. In some embodiments, the linker is attached to the IL-2 polypeptide at residue 45.

In some embodiments, the linker is attached to the IL-2 polypeptide at a residue which disrupts binding of the IL-2 polypeptide with the IL-2 receptor alpha subunit (IL-2Rα). Examples of these residues include residues 3, 5, 34, 35, 36, 37, 38, 40, 41, 42, 43, 44, 45, 60, 61, 62, 63, 64, 65, 67, 68, 69, 71, 72, 103, 104, 105, and 107, as described in, for example, PCT Pub. Nos. WO2019028419A1, WO2020056066A1, WO2021140416A2, and WO2021216478A1 each of which is hereby incorporated by reference as if set forth in its entirety. In some embodiments, the linker is covalently attached at a residue selected from residues corresponding to residues 3, 34, 35, 36, 37, 38, 40, 41, 42, 43, 44, 45, 60, 61, 62, 63, 64, 65, 67, 68, 69, 71, 72, 103, 104, 105, and 107 of SEQ ID NO: 301. In some embodiments, the linker is covalently attached at residue 1, 35, 37, 38, 41, 42, 43, 44, 45, 60, 61, 62, 64, 65, 68, 69, 71, 72, 104, 105, or 107 of the IL-2 polypeptide In some embodiments, the linker is covalently attached at residue 5. In some embodiments, the linker is covalently attached at residue 38. In some embodiments, the linker is covalently attached at residue 42. In some embodiments, the linker is covalently attached at residue 45. In some embodiments, the linker is covalently attached at residue 61. In some embodiments, the linker is covalently attached at residue 65. In some embodiments, the linker is covalently attached at residue 68.

In some embodiments, the residue to which the linker is attached is a natural amino acid residue. In some embodiments, the residue to which the linker is covalently attached is selected from cysteine, aspartate, asparagine, glutamate, glutamine, serine, threonine, lysine, and tyrosine. In some embodiments, the residue to which the linker is covalently attached is selected from asparagine, aspartic acid, cysteine, glutamic acid, glutamine, lysine, and tyrosine. In some embodiments, the linker is covalently attached to a cysteine. In some embodiments, the linker is covalently attached to a lysine. In some embodiments, the linker is covalently attached to a glutamine. In some embodiments, the linker is covalently attached to an asparagine. In some embodiments, the residue to which the linker is attached is a tyrosine. In some embodiments, the residue to which the linker is attached is the natural amino acid in that position in SEQ ID NO: 301.

In some embodiments, the linker is attached to a different natural amino acid which is substituted at the relevant position. The substitution can be for a naturally occurring amino acid which is more amenable to attachment of additional functional groups (e.g., aspartic acid, cysteine, glutamic acid, lysine, serine, threonine, or tyrosine), a derivative of modified version of any naturally occurring amino acid, or any unnatural amino acid (e.g., an amino acid containing a desired reactive group, such as a CLICK chemistry reagent such as an azide, alkyne, etc.). In some embodiments, the linker is covalently attached to site-specifically to a natural amino acid.

In some embodiments, the linker is attached at an unnatural amino acid residue. In some embodiments, the unnatural amino acid residue comprises a conjugation handle. In some embodiments, the conjugation handle facilitates the addition of the linker to the modified IL-18 polypeptide. The conjugation handle can be any of the conjugation handles provided herein. In some embodiments, the linker is covalently attached site-specifically to the unnatural amino acid. Non-limiting examples of amino acid residues comprising conjugation handles can be found, for example, in PCT Pub. Nos. WO2015054658A1, WO2014036492A1, and WO2021133839A1 WO2006069246A2, and WO2007079130A2, each of which is incorporated by reference as if set forth in its entirety.

In some embodiments, the linker is covalently attached at residue 42. In some embodiments, the linker is covalently attached at residue F42E, F42D, F42Q, F42K, F42N, or F42Y. In some embodiments, the linker is covalently attached at residue F42Y. In some embodiments, the linker is covalently attached to an unnatural amino acid at residue 42.

In some embodiments, the linker is covalently attached at residue 45. In some embodiments, the linker is covalently attached at residue Y45, Y45E, Y45C, Y45D, Y45Q, Y45K, or Y45N. In some embodiments, the linker is covalently attached at residue Y45. In some embodiments, the linker is covalently attached to an unnatural amino acid at residue 45.

In some embodiments, the linker is covalently attached at residue 65. In some embodiments, the linker is covalently attached at residue P65C, P65D, P65Q, P65E, P65N, P65K, or P65Y. In some embodiments, the linker is covalently attached residue P65C. In some embodiments, the linker is covalently attached to an unnatural amino acid at residue 65.

In some embodiments, the linker is covalently attached at residue 5. In some embodiments, the linker is covalently attached at residue S5C, S5D, S5Q, S5K, S5N, S5K, or S5Y. In some embodiments, the linker is covalently attached residue S5C. In some embodiments, the linker is covalently attached to an unnatural amino acid at residue 5.

In some embodiments, the linker is covalently attached through a modified natural amino acid. In some embodiments, the modified natural amino acid comprises a conjugation handle. In some embodiments, the linker is covalently attached through a modified amino acid α. In some embodiments, the modified amino acid α is an amino-acid-PEG-azide group or an amino-acid-PEG-alkyne group. In some embodiments, the modified amino acid α is a glutamate, aspartate, lysine, cysteine, or tyrosine modified to incorporate an azide, alkyne, or other conjugation handle group linked to the amino acid through a PEG spacer. In some embodiments, the modified amino acid α has a structure selected from:

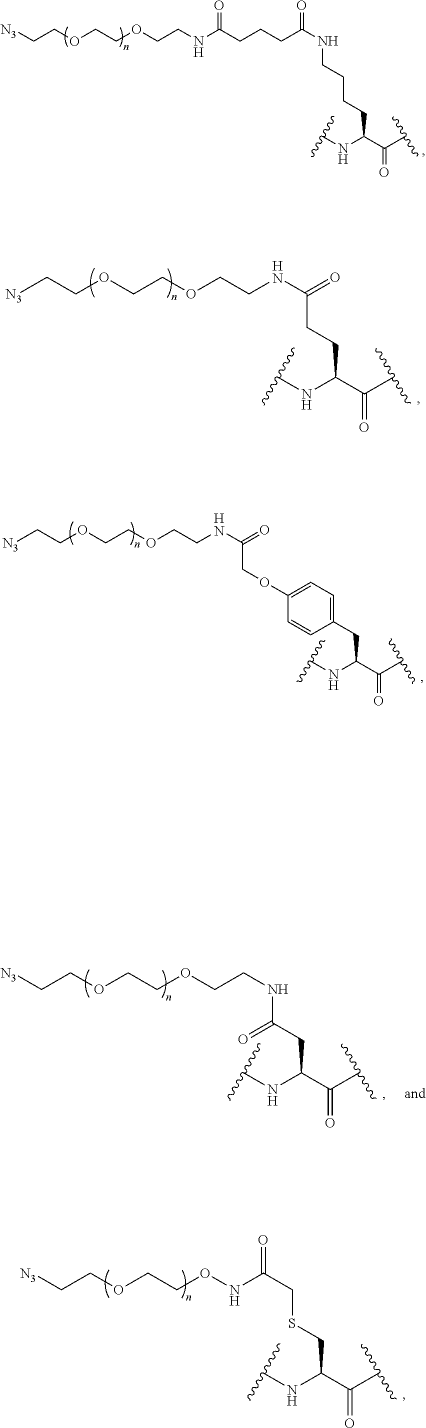

wherein each n is independently an integer from 1-30. In some embodiments, the modified amino acid α has a structure selected from:

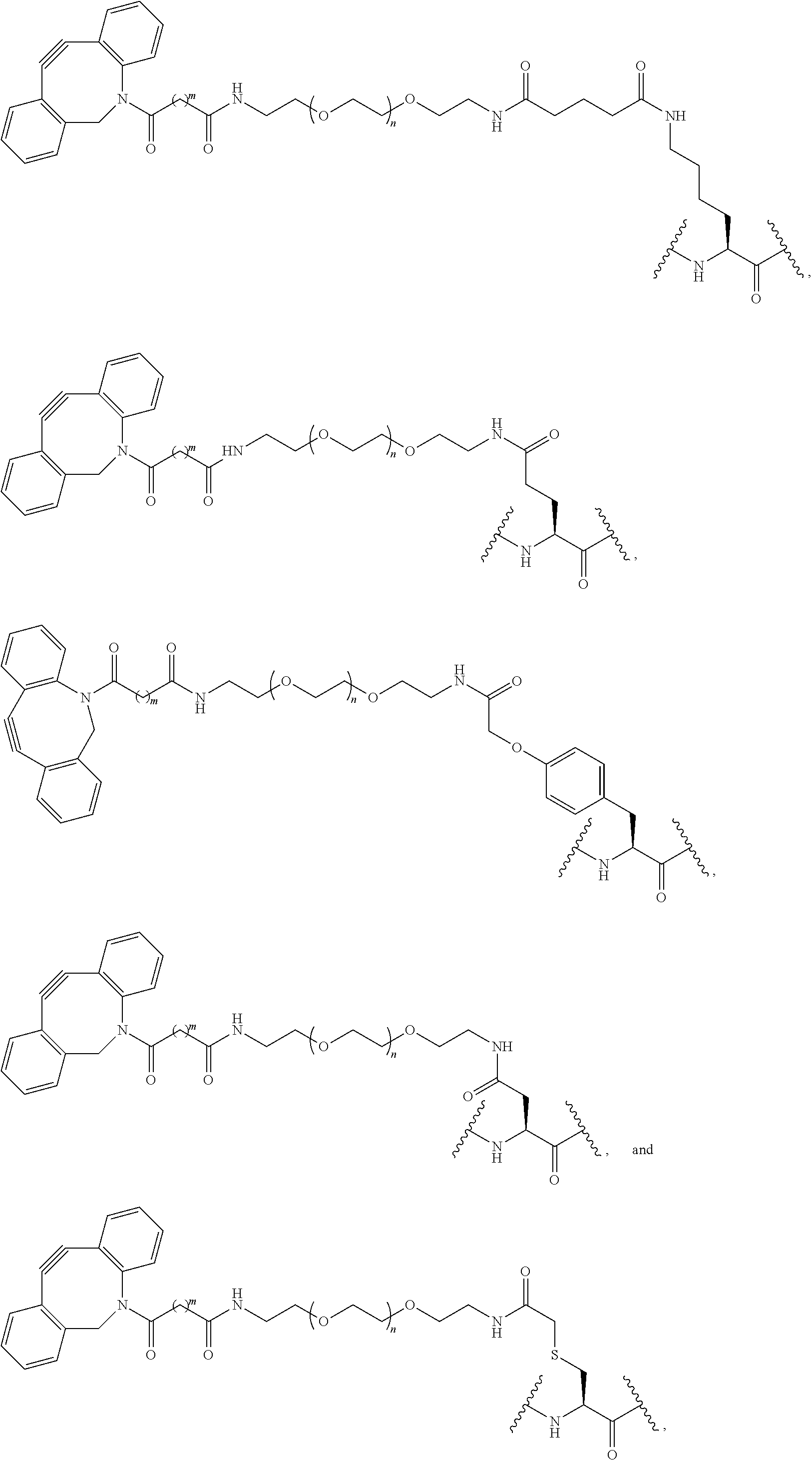

wherein each n is independently an integer from 1-30 and each m is independently an integer from 2-10. In some embodiments, n is an integer from 1-20, 1-10, 2-30, 2-20, 2-10, 5-30, 5-20, or 5-10. In some embodiments, n is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In some embodiments, n is 10. In some embodiments, n is 8. In some embodiments, n is 6. In some embodiments, n is 12. In some embodiments, each m is independently 2, 3, 4, 5, 6, 7, 8, 9, or 10. In some embodiments, m is 3. In some embodiments, m is 4. In some embodiments, m is 5. In some embodiments, m is 6. The modified amino acid α can be incorporated at any point of attachment of the IL-2 polypeptide as provided herein. In some embodiments, the modified amino acid α is located at a position on the modified IL-2 polypeptide selected from residue 5, residue 42, residue 45, or residue 65. In some embodiments, the modified amino acid α is located at residue 42 of the modified IL-2 polypeptide. In some embodiments, the modified amino acid α is located at residue 45 of the modified IL-2 polypeptide.

In some embodiments, the linker is attached to a terminal residue of the IL-2 polypeptide. In some embodiments, the linker is attached to the N-terminal residue of the IL-2 polypeptide. In some embodiments, the linker is attached to the N-terminal amine of the IL-2 polypeptide. In some embodiments, the linker is attached to the N-terminal amino of the IL-2 polypeptide through use of a conjugation handle attached to the N-terminal amine of the IL-2 polypeptide. In some embodiments, the linker is attached to the N-terminal amino of the IL-2 polypeptide through use of a conjugation handle attached to the N-terminal amine of the IL-2 polypeptide through a PEG group. In some embodiments, the conjugation handle comprises an azide or alkyne functionality. In some embodiments, the linker is attached to the N-terminal amine of the IL-2 polypeptide by use of a structure

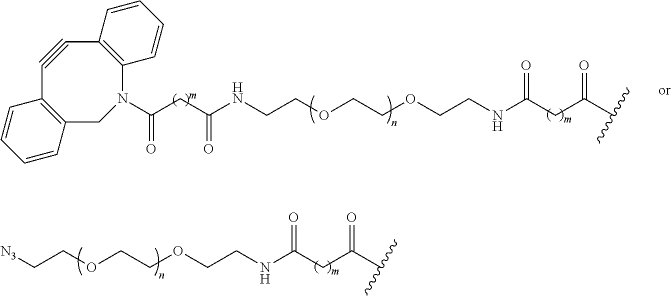

wherein each n is independently an integer from 1-30 and each m is independently an integer from 2-10. In some embodiments, n is an integer from 1-20, 1-10, 2-30, 2-20, 2-10, 5-30, 5-20, or 5-10. In some embodiments, n is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In some embodiments, n is 10. In some embodiments, n is 8. In some embodiments, n is 6. In some embodiments, n is 12. In some embodiments, each m is independently 2, 3, 4, 5, 6, 7, 8, 9, or 10. In some embodiments, m is 3. In some embodiments, m is 4. In some embodiments, m is 5. In some embodiments, m is 6.

In some embodiments, the linker is attached to the C-terminal residue of the IL-2 polypeptide. In some embodiments, the linker is attached to the C-terminal carboxyl group of the Il-2 polypeptide. In some embodiments, the linker is attached to the C-terminal carboxyl of the IL-2 polypeptide through use of a conjugation handle attached to the C-terminal carboxyl of the IL-2 polypeptide. In some embodiments, the linker is attached to the C-terminal carboxyl of the IL-2 polypeptide through use of a conjugation handle attached to C-terminal carboxyl e of the IL-2 polypeptide through a PEG group. In some embodiments, the conjugation handle comprises an azide or alkyne functionality.

Where IL-2 polypeptides contain unnatural amino acids or modified natural amino acids (e.g., those provided herein for purposes of conjugation), these amino acids may be incorporated into the IL-2 polypeptides using many techniques known in the art for introduction such modifications. For example, recombinant proteins with unnatural amino acids can be made using methods as described in Patent Cooperation Treaty Publication Nos. WO2016115168, WO2002085923, WO2005019415, and WO2005003294. Alternatively or in combination, unnatural or modified natural amino acids can be incorporated into chemically synthesized proteins during synthesis Modifications to IL-2 Polypeptides

In some embodiments, the IL-2 polypeptide of the bifunctional cytokine composition comprises one or more modifications to that of SEQ ID NO: 301. The modifications provided herein are in addition to any modification at the point of attachment as discussed supra. In some embodiments, the residue position numbering of the modified IL-2 polypeptide is based on SEQ ID NO: 301 as a reference sequence.

Modifications to the IL-2 polypeptide described herein encompass mutations, addition of various functionalities, deletion of amino acids, addition of amino acids, or any other alteration of the wild-type version of the protein or protein fragment. Functionalities which may be added to polypeptides include polymers, linkers, alkyl groups, detectable molecules such as chromophores or fluorophores, reactive functional groups, or any combination thereof. In some embodiments, functionalities are added to individual amino acids of the polypeptides. In some embodiments, functionalities are added site-specifically to the polypeptides.

In some embodiments, the IL-2 polypeptide of the bifunctional cytokine composition comprise one or more modifications in addition to a modification needed to attach the linker to the relevant residue of the IL-2 polypeptide (e.g., an amino acid substitution at a residue to which the linker is not attached).

In some embodiments, the IL-2 polypeptide of the bifunctional cytokine composition described herein contains 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more modified amino acid residues.

In some embodiments, the IL-2 polypeptide of the bifunctional cytokine composition comprises an amino acid sequence having at least 80%, 85%, 90%, 91%, 92%, 93%, 94%, 95%, 96%, 97%, 98%, or 99% sequence identity to the sequence set forth in SEQ ID NO: 301.

In some embodiments, the IL-2 polypeptide of the bifunctional cytokine composition provided herein comprises an amino acid sequence of any one of SEQ ID NOs: 301-323 provided herein. In some embodiments, the IL-2 polypeptide comprises an amino acid sequence at least 85% identical to the sequence of any one of SEQ ID NOs: 301-323. In some embodiments, the IL-2 polypeptide comprises an amino acid sequence of SEQ ID NO: 303. In some embodiments, the IL-2 polypeptide comprises an amino acid sequence at least 85% identical to the sequence of SEQ ID NO: 303. In some embodiments, the IL-2 polypeptide comprises an amino acid sequence of SEQ ID NO: 307. In some embodiments, the IL-2 polypeptide comprises an amino acid sequence at least 85% identical to the sequence of SEQ ID NO: 307. In some embodiments, the IL-2 polypeptide comprises an amino acid sequence of SEQ ID NO: 302. In some embodiments, the IL-2 polypeptide comprises an amino acid sequence at least 85% identical to the sequence of SEQ ID NO: 302. In some embodiments, the IL-2 polypeptide of the bifunctional cytokine composition is derived from SEQ ID NO: 300 (e.g., SEQ ID NO: 300 is used as the base molecule to form the bifunctional cytokine composition via a reaction with the azide attached to residue 42).

In some embodiments, the IL-2 polypeptide of the bifunctional cytokine composition described herein comprises at least 1, at least 2, at least 3, at least 4, at least 5, at least 6, at least 7, or at least 9 amino acid substitutions, wherein the amino acid substitutions are relative to SEQ ID NO: 301. In some embodiments, the IL-2 polypeptide comprises 1 to 9 amino acid substitutions. In some embodiments, the IL-2 polypeptide comprises 1 or 2 amino acid substitutions, 1 to 3 amino acid substitutions, 1 to 4 amino acid substitutions, 1 to 5 amino acid substitutions, 1 to 6 amino acid substitutions, 1 to 7 amino acid substitutions, 1 to 8 amino acid substitutions, 2 to 3 amino acid substitutions, 2 to 4 amino acid substitutions, 2 to 5 amino acid substitutions, 2 to 6 amino acid substitutions, 2 to 7 amino acid substitutions, 2 to 8 amino acid substitutions, 2 to 9 amino acid substitutions 3 or 4 amino acid substitutions, 3 to 5 amino acid substitutions, 3 to 6 amino acid substitutions, 3 to 7 amino acid substitutions, 3 to 9 amino acid substitutions, 4 or 5 amino acid substitutions, 4 to 6 amino acid substitutions, 4 to 7 amino acid substitutions, 4 to 9 amino acid substitutions, 5 or 6 amino acid substitutions, 5 to 7 amino acid substitutions, 5 to 9 amino acid substitutions, 6 or 7 amino acid substitutions, 6 to 9 amino acid substitutions, or 7 to 9 amino acid substitutions. In some embodiments, the IL-2 polypeptide comprises 3 amino acid substitutions, 4 amino acid substitutions, 5 amino acid substitutions, 6 amino acid substitutions, 7 amino acid substitutions, or 9 amino acid substitutions. In some embodiments, the IL-2 polypeptide comprises at most 4 amino acid substitutions, 5 amino acid substitutions, 6 amino acid substitutions, 7 amino acid substitutions, or 9 amino acid substitutions.

In some embodiments, the IL-2 polypeptide comprising of the bifunctional cytokine composition described herein comprises at least 1, at least 2, at least 3, at least 4, at least 5, at least 6, at least 7, or at least 9 natural amino acid substitutions, wherein the natural amino acid substitutions are relative to SEQ ID NO: 301. In some embodiments, the IL-2 polypeptide comprises 1 to 9 natural amino acid substitutions. In some embodiments, the IL-2 polypeptide comprises 1 or 2 natural amino acid substitutions, 1 to 3 natural amino acid substitutions, 1 to 4 natural amino acid substitutions, 1 to 5 natural amino acid substitutions, 1 to 6 natural amino acid substitutions, 1 to 7 natural amino acid substitutions, 1 to 8 natural amino acid substitutions, 2 to 3 natural amino acid substitutions, 2 to 4 natural amino acid substitutions, 2 to 5 natural amino acid substitutions, 2 to 6 natural amino acid substitutions, 2 to 7 natural amino acid substitutions, 2 to 8 natural amino acid substitutions, 2 to 9 natural amino acid substitutions, 3 or 4 natural amino acid substitutions, 3 to 5 natural amino acid substitutions, 3 to 6 natural amino acid substitutions, 3 to 7 natural amino acid substitutions, 3 to 9 natural amino acid substitutions, 4 or 5 natural amino acid substitutions, 4 to 6 natural amino acid substitutions, 4 to 7 amino acid substitutions, 4 to 9 natural amino acid substitutions, 5 or 6 natural amino acid substitutions, 5 to 7 amino acid substitutions, 5 to 9 natural amino acid substitutions, 6 or 7 natural amino acid substitutions, 6 to 9 natural amino acid substitutions, or 7 to 9 natural amino acid substitutions. In some embodiments, the IL-2 polypeptide comprises 3 natural amino acid substitutions, 4 natural amino acid substitutions, 5 amino acid substitutions, 6 natural amino acid substitutions, 7 natural amino acid substitutions, or 9 natural amino acid substitutions. In some embodiments, the IL-2 polypeptide comprises at most 4 natural amino acid substitutions, 5 natural amino acid substitutions, 6 natural amino acid substitutions, 7 natural amino acid substitutions, or 9 natural amino acid substitutions. In some embodiments, the IL-2 polypeptide further comprises up to 10 non-canonical amino acid substitutions. In some embodiments, the IL-18 polypeptide comprises 1, 2, 3, 4, 5, 6, 7, 8, 9 or 10 unnatural amino acid substitutions. In some embodiments, the IL-2 polypeptide further comprises unnatural amino acid substitutions at residues M23, M39, and/or M46. In some embodiments, the unnatural amino acid residues substituted for the methionines are each independently norleucine or O-methyl-homoserine. In some embodiments, the IL-2 polypeptide further comprises unnatural amino acid substitutions at residues 41, 71, and 104. In some embodiments, the IL-18 polypeptide further comprises homoserine (Hse) 41, Hse 71, and Hse 104.

In some embodiments, the IL-2 polypeptide of the bifunctional cytokine composition described herein comprises at least 1, at least 2, at least 3, at least 4, at least 5, at least 6, at least 7, or at least 9 amino acid substitutions, wherein the amino acid substitutions are relative to SEQ ID NO: 304. In some embodiments, the IL-2 polypeptide comprises 1 to 9 amino acid substitutions. In some embodiments, the IL-2 polypeptide comprises 1 or 2 amino acid substitutions, 1 to 3 amino acid substitutions, 1 to 4 amino acid substitutions, 1 to 5 amino acid substitutions, 1 to 6 amino acid substitutions, 1 to 7 amino acid substitutions, 1 to 8 amino acid substitutions, 2 to 3 amino acid substitutions, 2 to 4 amino acid substitutions, 2 to 5 amino acid substitutions, 2 to 6 amino acid substitutions, 2 to 7 amino acid substitutions, 2 to 8 amino acid substitutions, 2 to 9 amino acid substitutions 3 or 4 amino acid substitutions, 3 to 5 amino acid substitutions, 3 to 6 amino acid substitutions, 3 to 7 amino acid substitutions, 3 to 9 amino acid substitutions, 4 or 5 amino acid substitutions, 4 to 6 amino acid substitutions, 4 to 7 amino acid substitutions, 4 to 9 amino acid substitutions, 5 or 6 amino acid substitutions, 5 to 7 amino acid substitutions, 5 to 9 amino acid substitutions, 6 or 7 amino acid substitutions, 6 to 9 amino acid substitutions, or 7 to 9 amino acid substitutions. In some embodiments, the IL-2 polypeptide comprises 3 amino acid substitutions, 4 amino acid substitutions, 5 amino acid substitutions, 6 amino acid substitutions, 7 amino acid substitutions, or 9 amino acid substitutions. In some embodiments, the IL-2 polypeptide comprises at most 4 amino acid substitutions, 5 amino acid substitutions, 6 amino acid substitutions, 7 amino acid substitutions, or 9 amino acid substitutions.

In some embodiments, the IL-2 polypeptide comprises at least one substitution or modification (e.g., attachment of a polymer) to the amino acid sequence of SEQ ID NO: 301. In some embodiments, the at least one substitution or modification has an impact on the ability of the IL-2 polypeptide to bind to one or more IL-2 receptor subunits. In some embodiments, the at least one substitution or modification diminishes the ability of the IL-2 polypeptide to bind to the IL-2 receptor a subunit. Non-limiting examples such modifications are described in, for example, PCT Publication Nos. WO2021140416A2, WO2012065086A1, WO2019028419A1, WO2012107417A1, WO2018119114A1, WO2012062228A2, WO2019104092A1, WO2012088446A1, and WO2015164815A1, each of which is hereby incorporated by reference as if set forth herein in its entirety. In addition to modifications of IL-2 which may affect binding to one or more IL-2 receptor subunits (such as the alpha subunit), the IL-2 polypeptide provided herein may also comprises one or more modifications which improve the stability or pharmacokinetic properties of the IL-2 polypeptide. For example, the IL-2 polypeptide provided herein can comprise the modifications relative to SEQ NO: 301 which are contained in aldesluekin (Proleukin®) (SEQ ID NO: 302), namely a deletion of the N-terminal A residue of WT IL-2 and a C125S substitution relative to WT IL-2.

Non-limiting examples of modifications to IL-2 polypeptides include amino acid substitutions shown in Table 1 below. In some embodiments, the IL-2 polypeptide comprises 1, 2, 3, 4, 5, or more of the amino acid substitutions set forth in Table 1.

TABLE 1

| WT IL-2 | | |
|---|---|---|
| Residue | WT IL-2 | |
| Number* | Residue | Mutations |
| 35 | K | D, I, L, M, N, P, Q, T, Y |
| 36 | L | A, D, E, F, G, H, I, K, M, N, P, R, S, |
| | | W, Y |
| 38 | R | A, D, G, K, N, P, S, Y |
| 40 | L | D, G, N, S, Y |
| 41 | T | E, G, Y |
| 42 | F | A, D, E, G, I, K, L, N, Q, R, S, T, |
| | | V, Y |
| 43 | K | H, Y |
| 44 | F | K, Y |
| 45 | Y | A, D, E, G, K, L, N, Q, R, S, T, V |
| 46 | M | I, Y |
| 61 | E | K, M, R, Y |
| 62 | E | D, L, T, Y |
| 64 | K | D, E, G, L, Q, R, Y |
| 65 | P | D, E, F, G, H, I, K, L, N, Q, R, S, T, |
| | | V, W, Y |
| 66 | L | A, F, Y |
| 67 | E | A, Y |
| 68 | E | V, Y |
| 72 | L | A, D, E, G, K, N, Q, R, S, T, Y |
| 125 | C | S |

*Residue position numbering based on SEQ ID NO: 301 as a reference sequence

In some embodiments, the IL-2 polypeptide comprises 1, 2, 3, 4, 5, or more of the amino acid substitutions set forth in Table 2.

TABLE 2

| WT IL-2 | | |
|---|---|---|
| Residue | WT IL-2 | |
| Number* | Residue | Mutations |
| 20 | D | T, Y |
| 35 | K | D, I, L, M, N, P, Q, T Y |
| 38 | R | A, D, G, K, N, P, S, Y |
| 42 | F | A, D, E, G, I, K, L, N, Q, R, S, T, V, |
| | | Y |
| 43 | K | H, Y |
| 45 | Y | A, D, E, G, K, L, N, Q, R, S, T, V, Y |
| 62 | E | D, L, T, Y |
| 65 | P | D, E, F, G, H, I, K, L, N, Q, R, S, T, |
| | | V, W, Y |
| 68 | E | V, Y |
| 72 | L | A, D, E, G, K, N, Q, R, S, T, Y |
| 125 | C | S |

*Residue position numbering based on SEQ ID NO: 301 as a reference sequence

In some embodiments, the IL-2 polypeptide comprises at least one modification is in the range of amino acid residues 30-75. In some embodiments, the IL-2 polypeptide comprises at least one polymer attachment to the residue at position 42 and/or 45 and/or an amino acid substitution at residue position 42 and/or 45. In some embodiments, one modification is at amino acid residue 42. In some embodiments, one modification is a F42Y substitution. In some embodiments, one modification is a polymer attached to residue F42Y. In some embodiments, one modification is at residue 45. In some embodiments, the modification at residue 45 is a polymer attached to residue 45. In some embodiments, the modification at residue 45 is a polymer attached to residue Y45. In some embodiments, the IL-2 polypeptide comprises a first polymer attached at residue F42Y and a second polymer attached at residue Y45. In some embodiments, the IL-2 polypeptide comprises a deletion of residue 1 from SEQ ID NO: 301. In some embodiments, the IL-2 polypeptide comprises a C125S substitution. In some embodiments, the IL-2 polypeptide further comprises one or more substitutions of a synthetic IL-2 polypeptide as provided herein (e.g., Hse or Nle substitutions).

In one aspect, provided herein, is a modified IL-2 polypeptide of a bifunctional cytokine composition as provided herein (e.g., with a linker attached to a residue as provided herein, such as the N-terminal residue), further comprising a first polymer covalently attached at residue 42 and a second polymer covalently attached at residue 45, wherein residue position numbering of the IL-2 polypeptide is based on SEQ ID NO: 301 as a reference sequence. In some embodiments, the first polymer and the second polymer are the same. In some embodiments, the first polymer and the second polymer are different. In some embodiments, each polymer is attached through a tyrosine residue. In some embodiments, the IL-2 polypeptide comprises an amino acid sequence at least 80%, at least 85%, at least 90%, at least 95%, or at least 98% identical to the amino acid sequence of SEQ ID NO: 301. In some embodiments, the IL-2 polypeptide comprises an amino acid sequence at least 80%, at least 85%, at least 90%, at least 95%, or at least 98% identical to the amino acid sequence of SEQ ID NO: 302. In some embodiments, the IL-2 polypeptide comprises a C125S or C125A substitution. In some embodiments, the IL-2 polypeptide comprises a deletion of A1 from the sequence of SEQ ID NO: 301. In some embodiments, the IL-2 polypeptide comprises amino acid substitutions at 1, 2, 3, or 4 methionine residues from SEQ ID NO: 301. In some embodiments, the IL-2 polypeptide further comprises unnatural amino acid substitutions at residues M23, M39, and/or M46. In some embodiments, the unnatural amino acid residues substituted for the methionines are each independently norleucine or O-methyl-homoserine. In some embodiments, the IL-2 polypeptide further comprises homoserine Hse 41, Hse 71, and Hse 104.

In some embodiments, the IL-2 polypeptide comprises a polypeptide sequence having at least about 80%, at least about 85%, at least about 90%, at least about 95%, or about 100% sequence identity to SEQ ID NO: 301-323. In some embodiments, the polypeptide sequence is at least about 80% identical to SEQ ID NO: 303 or SEQ ID NO: 304. In some embodiments, the polypeptide sequence is at least about 80% identical to SEQ ID NO: 303. In some embodiments, the polypeptide sequence is at least about 90% identical to SEQ ID NO: 303. In some embodiments, the polypeptide sequence is at least about 95% identical to SEQ ID NO: 303. In some embodiments, the polypeptide sequence is at least about 80% identical to SEQ ID NO: 304. In some embodiments, the polypeptide sequence is at least about 90% identical to SEQ ID NO: 304. In some embodiments, the polypeptide sequence is at least about 95% identical to SEQ ID NO: 304. In some embodiments, the IL-2 polypeptide is synthetic.

### Polymers Attached to IL-2 Polypeptides

In some embodiments, the IL-2 polypeptide of the bifunctional cytokine composition comprises a polymer attached to a residue of the IL-2 polypeptide (e.g., a polymer in addition to the linker attached at the point of attachment). In some embodiments, the polymer is attached to a different residue than the residue to which the linker is attached.

In some embodiments, the polymer is attached to an amino acid residue of the IL-2 polypeptide. In some embodiments, the polymer is attached to any amino acid residue of the IL-2 polypeptide (e.g., at a position corresponding to any one of positions 1-133 of SEQ ID NO: 301). In some embodiments, the polymer is attached at a non-terminal residue (e.g., a residue other than the C-terminal residue or N-terminal residue) of the IL-2 polypeptide (e.g., a residue at position corresponding to any one of positions 2-132 of SEQ ID NO: 301). In some embodiments, the polymer is attached at a terminal residue of the IL-2 polypeptide, wherein the IL-2 polypeptide has been extended or truncated by one or more amino acids relative to SEQ ID NO: 301 (e.g., the linker is attached to a residue corresponding to residue 2 of SEQ ID NO: 301 and residue 1 of SEQ ID NO: 301 has been deleted). In some embodiments, the polymer is attached to the N-terminal residue of the IL-2 polypeptide. In some embodiments, the polymer is attached to the N-terminal amine of the IL-2 polypeptide. In some embodiments, the polymer is attached to the C-terminal residue of the IL-2 polypeptide. In some embodiments, the polymer is attached to the C-terminal carboxyl group of the IL-2 polypeptide.

In some embodiments, the polymer is attached to the IL-2 polypeptide at a residue in a region comprising residues 2-132, wherein residue position numbering is based on SEQ ID NO: 301 as a reference sequence. In some embodiments, the polymer is attached to the IL-2 polypeptide at a residue in a region comprising residues 30-75. In some embodiments, the polymer is attached to the IL-2 polypeptide at a residue in a region comprising residues 35-55, residues 35-50, residues 35-45, residues 30-50, residues 40-45, residues 60-75, residues 60-70, residues 65-70, or residues 2-5. In some embodiments, the polymer is attached to the IL-2 polypeptide at a residue selected from residue 65, 66, 67, 68, 69, and 70. In some embodiments, the polymer is attached to the IL-2 polypeptide at a residue selected from residue 40, 41, 42, 43, 44, and 45. In some embodiments, the polymer is attached to the IL-2 polypeptide at residue 42 or 45. In some embodiments, the polymer is attached to the IL-2 polypeptide at residue 42. In some embodiments, the polymer is attached to the IL-2 polypeptide at residue 45.

In some embodiments, the polymer is attached to the IL-2 polypeptide at a residue which disrupts binding of the IL-2 polypeptide with the IL-2 receptor alpha subunit (IL-2R). Examples of these residues include residues 3, 5, 34, 35, 36, 37, 38, 40, 41, 42, 43, 44, 45, 60, 61, 62, 63, 64, 65, 67, 68, 69, 71, 72, 103, 104, 105, and 107, as described in, for example, PCT Pub. Nos. WO2019028419A1, WO2020056066A1, WO2021140416A2, and WO2021216478A1 each of which is hereby incorporated by reference as if set forth in its entirety. In some embodiments, the polymer is covalently attached at a residue selected from residues corresponding to residues 3, 34, 35, 36, 37, 38, 40, 41, 42, 43, 44, 45, 60, 61, 62, 63, 64, 65, 67, 68, 69, 71, 72, 103, 104, 105, and 107 of SEQ ID NO: 301. In some embodiments, the polymer is covalently attached at residue 1, 35, 37, 38, 41, 42, 43, 44, 45, 60, 61, 62, 64, 65, 68, 69, 71, 72, 104, 105, or 107 of the IL-2 polypeptide. In some embodiments, the polymer is covalently attached at residue 5. In some embodiments, the polymer is covalently attached at residue 38. In some embodiments, the polymer is covalently attached at residue 42. In some embodiments, the polymer is covalently attached at residue 45. In some embodiments, the polymer is covalently attached at residue 61. In some embodiments, the polymer is covalently attached at residue 65. In some embodiments, the polymer is covalently attached at residue 68.

In some embodiments, the residue to which the polymer is attached is a natural amino acid residue. In some embodiments, the residue to which the polymer is covalently attached is selected from cysteine, aspartate, asparagine, glutamate, glutamine, serine, threonine, lysine, and tyrosine. In some embodiments, the residue to which the polymer is covalently attached is selected from asparagine, aspartic acid, cysteine, glutamic acid, glutamine, lysine, and tyrosine. In some embodiments, the polymer is covalently attached to a cysteine. In some embodiments, the polymer is covalently attached to a lysine. In some embodiments, the polymer is covalently attached to a glutamine. In some embodiments, the polymer is covalently attached to an asparagine. In some embodiments, the residue to which the polymer is attached is a tyrosine. In some embodiments, the residue to which the polymer is attached is the natural amino acid in that position in SEQ ID NO: 301 (e.g., Y45 or A1).

In some embodiments, the polymer is attached to a different natural amino acid which is substituted at the relevant position. The substitution can be for a naturally occurring amino acid which is more amenable to attachment of additional functional groups (e.g., aspartic acid, cysteine, glutamic acid, lysine, serine, threonine, or tyrosine), a derivative of modified version of any naturally occurring amino acid, or any unnatural amino acid (e.g., an amino acid containing a desired reactive group, such as a CLICK chemistry reagent such as an azide, alkyne, etc.). In some embodiments, the 1 polymer is covalently attached to site-specifically to a natural amino acid.

In some embodiments, the polymer is attached to a tyrosine residue. In some embodiments, the polymer attached to the tyrosine residue has a structure

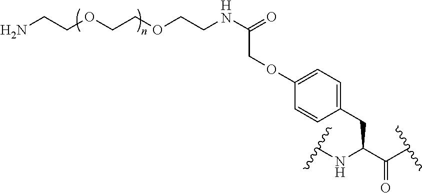

wherein n is an integer from 1-30. In some embodiments, n is an integer from 1-20, 1-10, 2-30, 2-20, 2-10, 5-30, 5-20, or 5-10. In some embodiments, n is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In some embodiments, n is 10. In some embodiments, n is 8. In some embodiments, n is 6. In some embodiments, n is 12. In some embodiments, the polymer attached to the tyrosine residue is at residue F42Y. In some embodiments, the polymer attached to the tyrosine residue is at Y45. In some embodiments, the IL-2 polypeptide comprises two polymers attached to tyrosine residues at F42Y and Y45. In some embodiments, the two polymers are the same size.

In some embodiments, the polymer is attached at an unnatural amino acid residue. In some embodiments, the unnatural amino acid residue comprises a conjugation handle. In some embodiments, the conjugation handle facilitates the addition of the polymer to the modified IL-2 polypeptide. The conjugation handle can be any of the conjugation handles provided herein, and is preferably a different conjugation handle which is non-reactive with a conjugation handle used to attach or form part of the linker (where a conjugation handle is used to form the linker). In some embodiments, the polymer is covalently attached site-specifically to the unnatural amino acid. Non-limiting examples of amino acid residues comprising conjugation handles can be found, for example, in PCT Pub. Nos. WO2015054658A1, WO2014036492A1, and WO2021133839A1 WO2006069246A2, and WO2007079130A2, each of which is incorporated by reference as if set forth in its entirety. In some embodiments, the polymer is attached to an unnatural amino acid residue without use of a conjugation handle.

In some embodiments, the polymer is covalently attached at residue 42. In some embodiments, the polymer is covalently attached at residue F42E, F42D, F42Q, F42K, F42N, or F42Y. In some embodiments, the polymer is covalently attached at residue F42Y. In some embodiments, the polymer is covalently attached to an unnatural amino acid at residue 42.

In some embodiments, the polymer is covalently attached at residue 45. In some embodiments, the polymer is covalently attached at residue Y45, Y45E, Y45C, Y45D, Y45Q, Y45K, or Y45N. In some embodiments, the polymer is covalently attached at residue Y45. In some embodiments, the polymer is covalently attached to an unnatural amino acid at residue 45.

In some embodiments, the polymer is covalently attached at residue 65. In some embodiments, the polymer is covalently attached at residue P65C, P65D, P65Q, P65E, P65N, P65K, or P65Y. In some embodiments, the polymer is covalently attached to an unnatural amino acid at residue 65.

In some embodiments, the polymer is covalently attached at residue 5. In some embodiments, the polymer is covalently attached at residue S5C, S5D, S5Q, S5K, S5N, S5K, or S5Y. In some embodiments, the polymer is covalently attached to an unnatural amino acid at residue 5.

In some embodiments, the polymer is covalently attached at residue 1. In some embodiments, the polymer is covalently attached at residue A1. In some embodiments, the polymer is covalently attached to the N-terminal amine of the IL-2 polypeptide.

In some embodiments, the polymer comprises a water-soluble polymer. In some embodiments, the water-soluble polymer comprises poly(alkylene oxide), polysaccharide, poly(vinyl pyrrolidone), poly(vinyl alcohol), polyoxazoline, poly(acryloylmorpholine), or a combination thereof. In some embodiments, the water-soluble polymer is poly(alkylene oxide). In some embodiments, the water-soluble polymer is polysaccharide. In some embodiments, the water-soluble polymer is poly(ethylene oxide) (PEG).

In some embodiments, the polymer has a molecular weight of from about 0.1 kDa to about 50 kDa. In some embodiments, the polymer has a molecular weight of from about 0.1 kDa to about 0.5 kDa from about 0.1 kDa to about 1 kDa, from about 0.1 kDa to about 2 kDa, from about 0.1 kDa to about 5 kDa from about 0.2 kDa to about 1 kDa, from about 0.2 kDa to about 2 kDa, from about 0.2 kDa to about 5 kDa, from about 0.2 kDa to about 10 kDa, from about 0.2 kDa to about 30 kDa, from about 0.5 kDa to about 2 kDa, from about 0.5 kDa to about 5 kDa, from about 0.5 kDa to about 10 kDa, from about 0.5 kDa to about 30 kDa, from about 1 kDa to about 5 kDa, from about 1 kDa to about 10 kDa, from about 1 kDa to about 30 kDa, from about 1 kDa to about 50 kDa, from about 2 kDa to about 10 kDa, from about 2 kDa to about 30 kDa, from about 2 kDa to about 50 kDa, from about 5 kDa to about 30 kDa, or from about 5 kDa to about 50 kDa. In some embodiments, the polymer has a molecular weight of at least about 0.2 kDa, at least about 0.5 kDa, at least about 1 kDa, at least about 2 kDa, at least about 5 kDa, at least about 10 kDa, or at least about 30 kDa. In some embodiments, the polymer has a molecular weight of at most about 30 kDa, at most about 10 kDa, at most about 5 kDa, at most about 2 kDa, at most about 1 kDa, at most about 0.5 kDa or at most about 0.2 kDa. In some embodiments, the polymer has a molecular weight of about 0.5 kDa, about 1 kDa, about 2 kDa, about 3 kDa, about 4 kDa, about 5 kDa, about 7.5 kDa, about 10 kDa, about 12.5 kDa, about 15 kDa, about 20 kDa, about 25 kDa, about 30 kDa, about 35 KDa, about 40 kDa, about 45 kDa, or about 50 kDa. In some embodiments, the polymer is a PEG polymer.

In some embodiments, the polymer is linear. In some embodiments, the polymer is a linear PEG polymer. In some embodiments, the polymer is branched. In some embodiments, the polymer is a branched PEG polymer. In some embodiments, the branched PEG polymer comprises a plurality of PEG chains from a central source molecule (e.g., a lysine or poly-lysine source molecule). In some embodiments, the polymer comprises from 1 to 10 polyethylene glycol chains. In some embodiments, the polymer comprises 1 polyethylene glycol chains to 10 polyethylene glycol chains. In some embodiments, the polymer comprises 1 polyethylene glycol chains to 2 polyethylene glycol chains, 1 polyethylene glycol chains to 4 polyethylene glycol chains, 1 polyethylene glycol chains to 6 polyethylene glycol chains, 1 polyethylene glycol chains to 10 polyethylene glycol chains, 2 polyethylene glycol chains to 4 polyethylene glycol chains, 2 polyethylene glycol chains to 6 polyethylene glycol chains, 2 polyethylene glycol chains to 10 polyethylene glycol chains, 4 polyethylene glycol chains to 6 polyethylene glycol chains, 4 polyethylene glycol chains to 10 polyethylene glycol chains, or 6 polyethylene glycol chains to 10 polyethylene glycol chains. In some embodiments, the polymer comprises 1 polyethylene glycol chains, 2 polyethylene glycol chains, 4 polyethylene glycol chains, 6 polyethylene glycol chains, or 10 polyethylene glycol chains. In some embodiments, the first water-soluble polymer comprises at least 1 polyethylene glycol chains, 2 polyethylene glycol chains, 4 polyethylene glycol chains, or 6 polyethylene glycol chains. In some embodiments, the first water-soluble polymer comprises at most 2 polyethylene glycol chains, 4 polyethylene glycol chains, 6 polyethylene glycol chains, or 10 polyethylene glycol chains. In some embodiments, the polymer comprises 4 polyethylene glycol chains.

In some embodiments, the polymer is an end-capped polymer. In some embodiments, the polymer is an end-capped polyethylene glycol. In some embodiments, the polymer is end-capped with a functional group selected from amine, alkoxy (e.g., methoxy, ethoxy, propoxy, etc.), hydroxyl, amide (e.g., —NH(C═O)(C₁-C₄ alkyl), carboxylate, and ester (e.g., methyl ester, ethyl ester, etc.). In some embodiments, the polymer as an amine end-capped PEG.

In some embodiments, the IL-2 polypeptide comprises two polymer covalently attached to two separate residues of the IL-2 polypeptide. In some embodiments, the two polymers are a first polymer and a second polymer. Each of the first polymer and the second polymer can be attached to the IL-2 polypeptide at any of the residues as provided herein and can be any of the polymers provided herein (e.g., having any combination of sizes as provided herein). In some embodiments, both of the first polymer and the second polymer are the same size or about the same size. In some embodiments, both polymers are at most about 1 kDa. In some embodiments, one polymer is substantially larger than the other. In some embodiments, one polymer is at most about 1 kDa and the other polymer is at least about 5 kDa.

A non-limiting set of IL-2 polypeptides provided herein with various linker points of attachment and polymers as provided herein is shown in Table 3 below.

TABLE 3

| IL-2 | Linker Point of | Polymer 1 Point of | Polymer 2 Point of |
|---|---|---|---|
| Construct | Attachment | Attachment | Attachment |
| IL-2-A | N-terminus | Residue 42 | Residue 45 |
| IL-2-B | N-terminus | Residue 42 | None |
| IL-2-C | N-terminus | Residue 45 | None |
| IL-2-D | Residue 42 | Residue 45 | None |
| IL-2-E | Residue 42 | N-terminus | Residue 45 |
| IL-2-F | Residue 42 | N-terminus | None |
| IL-2-G | Residue 45 | Residue 42 | None |
| IL-2-H | Residue 45 | N-terminus | Residue 42 |
| IL-2-I | Residue 45 | N-terminus | None |
| IL-2-J | N-terminus | Residue 65 | None |
| IL-2-K | Residue 65 | N-terminus | None |

*Residue position numbering based on SEQ ID NO: 1 as a reference sequence

In some embodiments, the IL-2 polypeptide comprises the linker covalently attached to the N-terminus, a first polymer covalently attached at residue 42, and a second polymer covalently attached at residue 45. In some embodiments, the first polymer is covalently attached at residue F42Y. In some embodiments, the second polymer is covalently attached at residue Y45. In some embodiments, the first polymer and the second polymer are different sizes. In some embodiments, the first polymer has a molecular weight of at most about 1 kDa and the second polymer has a molecular weight of at least about 5 kDa. In some embodiments, the first polymer has a molecular weight of from about 0.1 kDa to about 1 kDa and the second polymer has a molecular weight of from about 5 kDa to about 50 kDa. In some embodiments, the first polymer has a molecular weight of at least about 5 kDa and the second polymer has a molecular weight of at most about 1 kDa. In some embodiments, the first polymer has a molecular weight of from about 5 kDa to about 50 kDa and the second polymer has a molecular weight of from about 0.1 kDa to about 1 kDa. In some embodiments, the first polymer and the second polymer are the same or about the same size. In some embodiments, the first polymer and the second polymer each have a molecular weight of from about 0.1 kDa to about 1 kDa, about 0.2 kDa to about 1 kDa, or from about 0.5 kDa to about 1 kDa.

In some embodiments, the IL-2 polypeptide comprises the linker covalently to residue 42, a first polymer covalently attached at residue 45, and a second polymer covalently attached at the N-terminus. In some embodiments, the linker is attached at residue F42Y. In some embodiments, the first polymer is covalently attached at residue Y45. In some embodiments, the first polymer and the second polymer are different sizes. In some embodiments, the first polymer has a molecular weight of at most about 1 kDa and the second polymer has a molecular weight of at least about 5 kDa. In some embodiments, the first polymer has a molecular weight of from about 0.1 kDa to about 1 kDa and the second polymer has a molecular weight of from about 5 kDa to about 50 kDa. In some embodiments, the first polymer has a molecular weight of at least about 5 kDa and the second polymer has a molecular weight of at most about 1 kDa. In some embodiments, the first polymer has a molecular weight of from about 5 kDa to about 50 kDa and the second polymer has a molecular weight of from about 0.1 kDa to about 1 kDa. In some embodiments, the first polymer and the second polymer are the same or about the same size. In some embodiments, the first polymer and the second polymer each have a molecular weight of from about 0.1 kDa to about 1 kDa, about 0.2 kDa to about 1 kDa, or from about 0.5 kDa to about 1 kDa.

In some embodiments, the IL-2 polypeptide comprises the linker covalently attached to residue 45, a first polymer covalently attached at residue 42, and a second polymer covalently attached at the N-terminus. In some embodiments, the first polymer is covalently attached at residue F42Y. In some embodiments, the linker is covalently attached at residue Y45. In some embodiments, the first polymer and the second polymer are different sizes. In some embodiments, the first polymer has a molecular weight of at most about 1 kDa and the second polymer has a molecular weight of at least about 5 kDa. In some embodiments, the first polymer has a molecular weight of from about 0.1 kDa to about 1 kDa and the second polymer has a molecular weight of from about 5 kDa to about 50 kDa. In some embodiments, the first polymer has a molecular weight of at least about 5 kDa and the second polymer has a molecular weight of at most about 1 kDa. In some embodiments, the first polymer has a molecular weight of from about 5 kDa to about 50 kDa and the second polymer has a molecular weight of from about 0.1 kDa to about 1 kDa. In some embodiments, the first polymer and the second polymer are the same or about the same size. In some embodiments, the first polymer and the second polymer each have a molecular weight of from about 0.1 kDa to about 1 kDa, about 0.2 kDa to about 1 kDa, or from about 0.5 kDa to about 1 kDa.

In some embodiments, the IL-2 polypeptide comprises the linker covalently attached to residue 45 and a polymer covalently attached at residue 42. In some embodiments, the linker is attached at residue Y45. In some embodiments, the polymer is attached at residue F42Y. In some embodiments, the polymer has a molecular weight of at most about 1 kDa. In some embodiments, the polymer has a molecular weight of from about 0.1 kDa to about 1 kDa. In some embodiments, the polymer has a molecular weight of at least about 5 kDa. In some embodiments, the polymer has a molecular weight of from about 5 kDa to about 50 kDa.

In some embodiments, the IL-2 polypeptide comprises the linker covalently attached to residue 42 and a polymer covalently attached at residue 45. In some embodiments, the linker is attached at residue F42Y. In some embodiments, the polymer is attached at residue Y45. In some embodiments, the polymer has a molecular weight of at most about 1 kDa. In some embodiments, the polymer has a molecular weight of from about 0.1 kDa to about 1 kDa. In some embodiments, the polymer has a molecular weight of at least about 5 kDa. In some embodiments, the polymer has a molecular weight of from about 5 kDa to about 50 kDa.

### Synthetic IL-2 Polypeptides

In some embodiments, the IL-2 polypeptide of the bifunctional cytokine composition provided herein is a synthetic IL-2 polypeptide. In some embodiments, the synthetic IL-2 polypeptide is prepared from one or more chemically synthesized fragments. Synthetic IL-2 polypeptides have been previously described, at least in PCT Publication No WO2021140416A2, US Patent Application Publication No US20210155665A1, and Asahina et al., Angew. Chem. Int. Ed. 2015, 54, 8226-8230, each of which is incorporated by reference as if set forth herein in its entirety.

Any IL-2 polypeptide provided herein may be prepared as a synthetic IL-2 polypeptide (e.g., having any of the amino acid substitutions (e.g., F42Y, C125S, etc.) or other modifications (e.g., polymer attachment) provided herein in conjunction with the substitutions provided herein for synthetic IL-2 polypeptides, such as homoserine or norleucine residues). In some embodiments, the IL-2 polypeptide comprises any of the amino acid substitutions present in a synthetic IL-2 polypeptide as provided herein (e.g., one or more homoserine or norleucine residues as provided herein). In some embodiments, a synthetic IL-2 polypeptide exhibits a similar or substantially identical activity to a corresponding recombinant IL-2 (e.g., a synthetic IL-2 polypeptide having the same functional modifications to the structure or sequence of the IL-2 polypeptide).

In some embodiments, the synthetic IL-2 polypeptide comprises a homoserine (Hse) residue located in any one of residues 35-45. In some embodiments, the synthetic IL-2 polypeptide comprises a Hse residue located in any one of residues 61-81. In some embodiments, the synthetic IL-2 polypeptide comprises a Hse residue located in any one of residues 94-114. In some embodiments, the synthetic IL-2 polypeptide comprises 1, 2, 3, or more Hse residues. In some embodiments, the synthetic IL-2 polypeptide comprises Hse41, Hse71, Hse104, or a combination thereof. In some embodiments, the synthetic IL-2 polypeptide comprises Hse41, Hse71, and Hse104. In some embodiments, the synthetic IL-2 polypeptide comprises at least two amino acid substitutions, wherein the at least two amino acid substitutions are selected from (a) a homoserine (Hse) residue located in any one of residues 35-45; (b) a homoserine residue located in any one of residues 61-81; and (c) a homoserine residue located in any one of residues 94-114. In some embodiments, the synthetic IL-2 polypeptide comprises Hse41 and Hse71. In some embodiments, the synthetic IL-2 polypeptide comprises Hse41 and Hse104. In some embodiments, the synthetic IL-2 polypeptide comprises Hse71 and Hse104. In some embodiments, the synthetic IL-2 polypeptide comprises Hse41. In some embodiments, the synthetic IL-2 polypeptide comprises Hse71. In some embodiments, the synthetic IL-2 polypeptide comprises Hse104. In some embodiments, the synthetic IL-2 polypeptide comprises 1, 2, 3, or more norleucine (Nle) residues. In some embodiments, the synthetic IL-2 polypeptide comprises a Nle residue located in any one of residues 18-28. In some embodiments, the synthetic IL-2 polypeptide comprises one or more Nle residues located in any one of residues 34-50. In some embodiments, the synthetic IL-2 polypeptide comprises a Nle residue located in any one of residues 20-60. In some embodiments, the synthetic IL-2 polypeptide comprises three Nle substitutions. In some embodiments, the synthetic IL-2 polypeptide comprises Nle23, Nle39, and Nle46.

### Biological Activity of IL-2 Polypeptides

In some embodiments, the bifunctional cytokine composition exhibits one or more activities associated with the IL-2 polypeptide of the bifunctional cytokine composition.

In some embodiments, the bifunctional cytokine composition retains binding to one or more IL-2 receptor subunits. In some embodiments, the bifunctional cytokine composition has an altered binding to one or more IL-2 receptor subunits relative to WT IL-2. In some embodiments, the bifunctional cytokine composition has a reduced binding to one or more IL-2 receptor subunits relative to WT IL-2. In some embodiments, the bifunctional cytokine composition has a substantially reduced binding to one IL-2 receptor subunit relative to WT IL-2.

In some embodiments, the bifunctional cytokine composition is biased towards the IL-2 receptor beta (IL-2Rβ) compared to WT IL-2. In some embodiments, the bifunctional cytokine composition exhibits an enhanced ability to signal through IL-2Rβ relative to IL-2Rα as compared to WT IL2.

In some embodiments, the bifunctional cytokine composition exhibits reduced binding to the IL-2 receptor alpha subunit (IL-2Rα). In some embodiments, the bifunctional cytokine composition exhibits binding to IL-2Rα which is reduced by at least about 10-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1000-fold, at least about 2000-fold, at least about 3000-fold, or at least about 5000-fold relative to WT IL-2. In some embodiments, the bifunctional cytokine composition does not exhibit substantial binding to IL-2Rα. In some embodiments, the bifunctional cytokine composition exhibits a reduced ability to signal through the IL-2 receptor alpha subunit. In some embodiments, the bifunctional cytokine composition exhibits an ability to signal through the IL-2 receptor alpha subunit which is reduced by at least 100-fold, 500-fold, 1000-fold, 2000-fold, 5000-fold, or 10000-fold compared to WT IL-2. In some embodiments, the bifunctional cytokine composition exhibits substantially no ability to signal through the IL-2 receptor alpha subunit.

In some embodiments, the bifunctional cytokine composition binds to the IL-2 receptor beta subunit (IL-2Rβ). In some embodiments, the bifunctional cytokine composition binds to IL-2Rβ with similar affinity to WT IL-2. In some embodiments, the affinity of the bifunctional cytokine composition to IL-2Rβ is within about 2-fold, within about 5-fold, within about 10-fold, within about 20-fold, within about 30-fold, within about 40-fold, or within about 50-fold of the affinity of WT IL-2 to IL-2Rβ. In some embodiments, the IL-2 polypeptide of the bifunctional cytokine composition binds to IL-2Rβ with an affinity which is at most about 2-fold lower than, at most about 5-fold lower then, or at most about 10-fold lower than the affinity with which WT IL-2 binds to IL-2Rβ. In some embodiment, the bifunctional cytokine composition retains the ability to signal through the IL-2 receptor beta subunit.

### Activity of IL-2/IL-18 Bifunctional Cytokine Compositions

In some embodiments, a bifunctional cytokine composition which contains an IL-2 polypeptide and an IL-18 polypeptide retains activities associated with both cytokines. In some embodiments, the linkage of the IL-2 polypeptide with the IL-18 polypeptide results in synergistic activity. In some embodiments, the bifunctional cytokine composition exhibits enhanced activity associated with one or both of the individual cytokines as compared to the corresponding cytokine alone (e.g., the IL-2 polypeptide not comprised in a bifunctional cytokine composition, or the IL-18 polypeptide not comprised in a bifunctional cytokine composition). In some embodiments, the bifunctional cytokine composition exhibits an activity which is enhanced compared to the IL-2 polypeptide and the IL-18 polypeptide together (e.g., co administered as individual cytokines).

In some embodiments, the bifunctional cytokine composition induces STAT5 phosphorylation in CD4⁺ T cells, CD8⁺ T cells, and/or NK cells more potently than a corresponding IL-2 polypeptide (e.g., the same IL-2 polypeptide of the bifunctional cytokine composition but not attached to the IL-18 polypeptide). In some embodiments, the bifunctional cytokine composition induces STAT5 phosphorylation in CD4⁺ T cells more potently than the corresponding IL-2 polypeptide. In some embodiments, the bifunctional cytokine composition induces STAT5 phosphorylation in CD4⁺ T cells at least 1.5-fold or at least 2-fold more potently than the corresponding IL-2 polypeptide (e.g., by comparing EC₅₀ values). In some embodiments, the bifunctional cytokine composition induces STAT5 phosphorylation in CD8⁺ T cells more potently than the corresponding IL-2 polypeptide. In some embodiments, the bifunctional cytokine composition induces STAT5 phosphorylation in CD8⁺ T cells at least 2-fold, 3-fold, 4-fold, or 5-fold more potently than the corresponding IL-2 polypeptide (e.g., by comparing EC₅₀ values). In some embodiments, the bifunctional cytokine composition induces STAT5 phosphorylation in NK cells more potently than the corresponding IL-2 polypeptide. In some embodiments, the bifunctional cytokine composition induces STAT5 phosphorylation in NK cells at least 2-fold, 5-fold, 10-fold, 15-fold, 20-fold, 25-fold, 30-fold, 35-fold, or 40-fold more potently than the corresponding IL-2 polypeptide. In some embodiments, the bifunctional cytokine composition induces STAT5 phosphorylation in each of CD4⁺ T cells, CD8⁺ T cells, and NK cells more potently than the corresponding IL-2 polypeptide. In some embodiments, the CD4⁺ T cells, CD8⁺ T cells, and/or NK cells are derived from PBMCs. In some embodiments, the effect is measured after 40 minutes of stimulation. In some embodiments, the bifunctional cytokine composition does not substantially induce STAT5 phosphorylation in B cells or dendritic cells.

In some embodiments, the bifunctional cytokine composition increases p65 levels in NK cells more potently than a corresponding IL-18 polypeptide. In some embodiments, the bifunctional cytokine composition increases p65 levels in NK cells to a greater degree than a corresponding IL-18 polypeptide. In some embodiments, the bifunctional cytokine composition does not substantially increase p65 levels in CD4⁺ T-cells, CD8⁺ T-cells, B cells, dendritic cells, or any combination thereof. In some embodiments, the cells are derived from PBMCs. In some embodiments, the effect is measured after 40 minutes of stimulation.

In some embodiments, the bifunctional cytokine composition induces greater IFNγ production than a corresponding IL-18 polypeptide. In some embodiments, the bifunctional cytokine composition induces at least 50%, 75%, 100%, 150%, 200%, 250%, or 300% greater IFNγ production than a corresponding IL-18 polypeptide. In some embodiments, the IFNγ production measured is the max level of IFNγ produced by the IL-18 polypeptide or the bifunctional cytokine composition. In some embodiments, the IFNγ production is measured at the EC₅₀ value of each of the bifunctional cytokine composition and the IL-18 polypeptide.

In some embodiments, the bifunctional cytokine composition exhibits an EC₅₀ of IFNγ production which is lower than for a corresponding IL-2 polypeptide. In some embodiments, the bifunctional cytokine composition exhibits an EC₅₀ of IFNγ production which is at least 1.5-fold, 2-fold, 3-fold, 5-fold, or 10-fold lower than for a corresponding IL-2 polypeptide.

In some embodiments, the bifunctional cytokine composition induces maximum levels of IFNγ production at levels comparable to a corresponding IL-2 polypeptide but with an EC₅₀ that is comparable to a corresponding IL-18 polypeptide. In some embodiments, the bifunctional cytokine composition induces greater maximum IFNγ production than a corresponding IL-18 polypeptide at an EC₅₀ which lower than for a corresponding IL-2 polypeptide.

### Interleukin 7 (IL-7) Cytokines and Derivatives Thereof

In some embodiments, at least one cytokine of a bifunctional cytokine composition provided herein is an IL-7 polypeptide. Interleukin-7 or a polypeptide having a similar activity thereto (hereinafter, “IL-7” or “IL7) refers to an immunostimulatory cytokine which can promote immune responses mediated by B cells and T cells. In particular, IL-7 plays an important role in an adaptive immune system. IL-7 is mostly secreted by stromal cells in the bone marrow and thymus, but it is also produced by keratinocytes, dendritic cells, hepatocytes, neurons, and epithelial cells. IL-7 activates immune functions through the survival, development, and differentiation of T cells and B cells, survival of lymphoid cells, stimulation of activity of natural killer (NK) cell, etc. IL-7 can regulate development of lymph nodes through lymphoid tissue inducer (LTi) cells, promotes the survival and division of naive T cells or memory T cells, maintains naive T cells or memory T cells, and enhances immune response in humans by inducing secretion of IL-2 and interferon-7. When a recombinant IL-7 is produced for the purpose of medicinal utilization, there are problems in that impurities increase compared to the general recombinant proteins, the amount of IL-7 degradation, and large-scale production cannot be easily achieved. However, since production of synthetic IL-7 requires a complicated denaturation process, the manufacturing process is not easy. Non-limiting examples of IL-7 amino acid sequences to be utilized in embodiments described herein are provided below in Table 4B. An IL-7 polypeptide utilized in conjugate described herein can have, for example, an amino acid sequence that is at least 80%, 85%, 90%, 91%, 92%, 93%, 94%, 95%, 96%, 97%, 98%, 99%, or 100% identical to any one of the sequences in Table 4B.

### Interleukin 10 (IL-10) Cytokines and Derivatives Thereof

In some embodiments, at least one cytokine of a bifunctional cytokine composition provided herein is an IL-10 polypeptide. In some embodiments, the IL-10 polypeptide comprises, an amino acid sequence that is at least 80%, 85%, 90%, 91%, 92%, 93%, 94%, 95%, 96%, 97%, 98%, 99%, or 100% identical to

(SEQ ID NO: 330)
MHSSALLCCL VLLTGVRASP GQGTQSENSC THFPGNLPNM
LRDLRDAFSR VKTFFQMKDQ LDNLLLKESL LEDFKGYLGC
QALSEMIQFY LEEVMPQAEN QDPDIKAHVN SLGENLKTLR
LRLRRCHRFL PCENKSKAVE QVKNAFNKLQ EKGIYKAMSE
FDIFINYIEA YMTMKIRN

### Interleukin 12 (IL-12) Cytokines and Derivatives Thereof

In some embodiments, at least one cytokine of a bifunctional cytokine composition provided herein is an IL-12 polypeptide. In some embodiments, the IL-12 polypeptide comprises, an amino acid sequence that is at least 80%, 85%, 90%, 91%, 92%, 93%, 94%, 95%, 96%, 97%, 98%, 99%, or 100% identical to

(SEQ ID NO: 329)
MCPARSLLLV ATLVLLDHLS LARNLPVATP DPGMFPCLHH
SQNLLRAVSN MLQKARQTLE FYPCTSEEID HEDITKDKTS
TVEACLPLEL TKNESCLNSR ETSFITNGSC LASRKTSFMM
ALCLSSIYED LKMYQVEFKT MNAKLLMDPK RQIFLDQNML
AVIDELMQAL NFNSETVPQK SSLEEPDFYK TKIKLCILLH
AFRIRAVTID RVMSYLNAS

### Uniformity of Bifunctional Cytokine Compositions

In some embodiments, bifunctional cytokine compositions provided herein are prepared in a manner which results in a population of the bifunctional cytokine compositions with a high level of uniformity. In some embodiments, a population of bifunctional cytokine composition consists of individual bifunctional cytokine compositions where all or nearly all of the individual bifunctional cytokines are the same (e.g., having the linker attached to the same residue in each of the cytokines of the bifunctional cytokine compositions). This is in contrast to methodologies which could be used to create bifunctional cytokine compositions in a non-site specific manner, such as by random attachment of heterobifunctional linking reagents to the surface of first cytokines followed by attachment of the heterobifunctional linking reagent to the surface of second cytokines. For example, if a cytokine comprising many surface exposed lysine residues was contacted with a heterobifunctional linking reagent comprising an N-hydroxysuccinimde (NHS) ester, each individual cytokine of a population would have the linking reagent attached at different residues, and potentially different numbers of the linking reagent attached. Using site specific attachment of the linker, as occurs some embodiments of the instant disclosure, thus provides for a more uniform final population of bifunctional cytokine compositions than that achieved over other linking strategies which could be employed to generate bifunctional cytokine compositions.

In some embodiments, a population of bifunctional cytokine compositions provided herein comprise a linker attached to a residue of a cytokine. In some embodiments, the linker is attached to at least about 80%, at least about 85%, at least about 90%, at least about 95%, at least about 96%, at least about 97%, at least about 98%, or at least about 99% of the individual bifunctional cytokine compositions at the same residue position of the cytokine. In some embodiments, the linker is also attached to at least about 80%, at least about 85%, at least about 90%, at least about 95%, at least about 96%, at least about 97%, at least about 98%, or at least about 99% of the individual bifunctional cytokine compositions at the same residue position of the second cytokine. the population comprises at least 10,000 individual bifunctional cytokine compositions. In some embodiments, the population comprises at least about 1 mg, at least about 10 mg, at least about 100 mg, or at least about 1 g of the bifunctional cytokine composition.

### Methods of Manufacturing

In one aspect, described herein, is a method of making a bifunctional cytokine composition. In some embodiments, the method comprises attaching a linker (e.g., a bifunctional linking reagent) to both the first cytokine and the second cytokine (for example, in a one-pot reaction). In some embodiments, the first cytokine and the second cytokine are reacted together directly (e.g., through conjugation handles affixed to the side chains of amino acids of the cytokines). In some embodiments, the first cytokine is reacted with a bifunctional linking reagent, then the first cytokine is reacted with the second cytokine. In some embodiments, the first cytokine is reacted with a first bifunctional linking reagent, the second cytokine is reacted with a second bifunctional linking reagent, then the first cytokine and the second cytokine are reacted to form the linker (e.g., through a reaction of the first bifunctional linking reagent and the second bifunctional linking reagent).

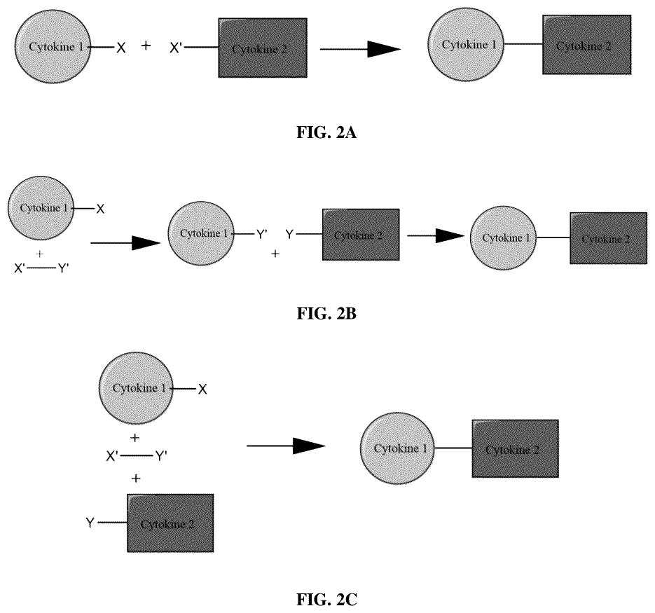

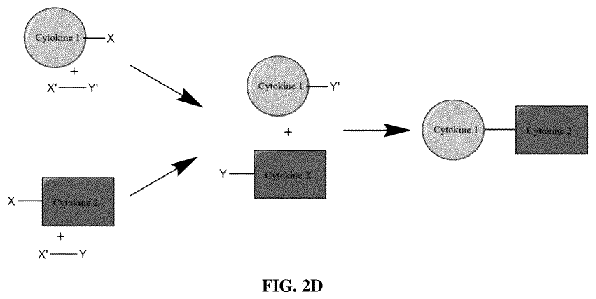

FIG. 2A illustrates one potential reaction scheme which can be used to conjugate a first cytokine and a second cytokine. A first cytokine comprising a conjugation handle X is contacted with a second cytokine comprising a complementary conjugation handle X′. The conjugation handle X is linked to a residue of the first cytokine. The conjugation handle X′ is linked to a residue of the second cytokine. The reaction proceeds as X reacts with X′ to complete the formation of the linker between the first cytokine and the second cytokine.

FIG. 2B illustrates another potential reaction scheme which can be used to conjugate a first cytokine and a second cytokine. In a first step, a first cytokine comprising a conjugation handle X (linked to a residue of the first cytokine) is reacted with a bifunctional linking reagent having a conjugation handle X′ (complementary to the conjugation handle X) and a conjugation handle Y′ (which is orthogonal to (e.g., unreactive with) the conjugation handle X. The result of this first step is that the first cytokine now comprises the conjugation handle Y′ linked to the residue of the first cytokine which contained the conjugation handle X. In a second step, the first cytokine is then reacted with a second cytokine comprising a conjugation handle Y which is complementary to the conjugation handle Y′. The conjugation handles Y and Y′ form a bond, thus forming the linker between the first cytokine and the second cytokine.

FIG. 2C illustrates another potential reaction scheme which can be used to conjugate a first cytokine and a second cytokine. In one reaction vessel, a first cytokine comprising a conjugation handle X linked to a residue of the cytokine, a second cytokine comprising a conjugation handle Y linked to a residue of the second cytokine, and a bifunctional linking reagent comprising a conjugation handle X′ (complementary to conjugation handle X) and a conjugation handle Y′ (complementary to conjugation handle Y) are combined. The bifunctional linking reagent reacts with the conjugation handles of the first and second cytokines to produce the bifunctional cytokine composition.

FIG. 2D illustrates another potential reaction scheme which can be used to conjugate a first cytokine and a second cytokine. In one reaction, a first cytokine comprising a conjugation handle X linked to a residue of the first cytokine is reacted with a bifunctional linking reagent comprising conjugation handles X′ and Y′, where X′ is complementary to conjugation handle X. After the reaction, the first cytokine comprises conjugation handle Y′ linked to a residue of the first cytokine. In a separate reaction, a second cytokine comprising a conjugation handle X linked to a residue of the second cytokine is reacted with a bifunctional linking reagent comprising conjugation handles X′ and Y, where X′ is complementary to conjugation handle X. After the reaction, the second cytokine comprises conjugation handle Y linked to a residue of the second cytokine. The reacted cytokines form the aforementioned reactions are then pooled (optionally with one or more purification steps). The conjugation handles Y and Y′ are complementary and reacted to complete formation of the linker between the first cytokine and the second cytokine in the bifunctional cytokine composition.

In one aspect, provided herein, is a method of making a bifunctional cytokine composition, wherein the bifunctional cytokine composition comprises a first cytokine, a second cytokine, and a linker comprising a first point of attachment to the first cytokine and a second point of attachment to the second cytokine; the method comprising: a) providing the first cytokine, wherein the first cytokine comprises a first cytokine conjugation handle; b) providing the second cytokine, wherein the second cytokine comprises a second cytokine conjugation handle; and forming a covalent bond through a reaction of the first cytokine conjugation handle with the second cytokine conjugation handle to form the linker.

In one aspect, provided herein, is a method of making a bifunctional cytokine composition, comprising: a) providing a first cytokine, wherein the first cytokine comprises a first cytokine conjugation handle; b) providing a second cytokine, wherein the second cytokine comprises a second cytokine conjugation handle; and forming a covalent bond through a reaction of the first cytokine conjugation handle with the second cytokine conjugation handle to form a linker.

In one aspect, provided herein, is a method of making a bifunctional cytokine composition, wherein the bifunctional cytokine composition comprises a first cytokine, a second cytokine, and a linker comprising a first point of attachment to the first cytokine and a second point of attachment to the second cytokine, the method comprising: a) providing the first cytokine, wherein the first cytokine comprises a first cytokine conjugation handle; b) providing the second cytokine, wherein the second cytokine comprises a second cytokine conjugation handle; c) providing a bifunctional linking reagent, wherein the bifunctional linking reagent comprises a first reagent conjugation handle and a second reagent conjugation handle; wherein the first reagent conjugation handle is complementary to the first cytokine conjugation handle, and wherein the second reagent conjugation handle is complementary to the second cytokine conjugation handle; d) forming a first covalent bond through a reaction of the first cytokine conjugation handle and the first reagent conjugation handle; and forming a second covalent bond through a reaction of the second cytokine conjugation handle and the second reagent conjugation handle, thereby forming the linker.

In one aspect, provided herein, is a method of making a bifunctional cytokine composition, comprising a) providing a first cytokine, wherein the first cytokine comprises a first cytokine conjugation handle; b) providing a second cytokine, wherein the second cytokine comprises a second cytokine conjugation handle; c) providing a bifunctional linking reagent, wherein the bifunctional linking reagent comprises a first reagent conjugation handle and a second reagent conjugation handle; wherein the first reagent conjugation handle is complementary to the first cytokine conjugation handle, and wherein the second reagent conjugation handle is complementary to the second cytokine conjugation handle; d) forming a first covalent bond through a reaction of the first cytokine conjugation handle and the first reagent conjugation handle; and forming a second covalent bond through a reaction of the second cytokine conjugation handle and the second reagent conjugation handle, thereby forming a linker between the first cytokine and the second cytokine.

The bifunctional cytokine composition made using the methods provided herein may be any of the bifunctional cytokine compositions provided herein (e.g., an IL-2/IL-18 bifunctional cytokine composition). The conjugation handles used in the methods may be any of the conjugation handles provided herein.

In some embodiments, providing a cytokine as provided herein (e.g., the first cytokine or the second cytokine) comprising synthesizing the cytokine. In some embodiments, synthesizing the cytokine comprises ligating one or more precursor fragments of the cytokine. In some embodiments, synthesizing the cytokine comprises incorporating an amino acid residue which contains a conjugation handle into the cytokine (e.g., during synthesis of a precursor fragment of the cytokine).

In some embodiments, a bifunctional linking reagent is used to link the first cytokine and the second cytokine. In some embodiments, a bifunctional linking reagent comprises two conjugation handles separated by a spacer moiety. Non-limiting examples of spacer moieties include poly(ethylene glycol) and other polymers, aliphatic hydrocarbon chains, and the like. In some embodiments, a bifunctional linking reagent is a heterobifunctional linking reagent (e.g., it contains two different conjugation handles, preferably which are not cross-reactive with each other). Many bifunctional linking reagents are known in the art and sold by commercial suppliers, including BroadPharm®, Santa Cruz Biotechnology, Inc., TCI America, Sigma-Aldrich, and ThermoFisher. Bifunctional linking reagents as described herein can also be referred to as “Bifunctional Linkers,” “Heterobifunctional Linkers,” “Bifunctional Crosslinkers,” and “Heterobifunctional Crosslinkers” in the art, but the term “bifunctional linking reagent” is used herein for clarity and differentiation from the “linker” of a bifunctional cytokine composition.

### Pharmaceutical Compositions

In one aspect, described herein is a pharmaceutical composition comprising: a bifunctional cytokine composition described herein; and a pharmaceutically acceptable carrier or excipient. In some embodiments, the pharmaceutical composition further comprises one or more excipients, wherein the one or more excipients include, but are not limited to, selected from a carbohydrate, an inorganic salt, an antioxidant, a surfactant, a buffer, or any combination thereof. In some embodiments the pharmaceutical composition further comprises one, two, three, four, five, six, seven, eight, nine, ten, or more excipients, wherein the one or more excipients include, but are not limited to, a carbohydrate, an inorganic salt, an antioxidant, a surfactant, a buffer, or any combination thereof.

In some embodiments, the pharmaceutical composition further comprises a carbohydrate. In certain embodiments, the carbohydrate is selected from the group consisting of fructose, maltose, galactose, glucose, D-mannose, sorbose, lactose, sucrose, trehalose, cellobiose raffinose, melezitose, maltodextrins, dextrans, starches, mannitol, xylitol, maltitol, lactitol, xylitol, sorbitol (glucitol), pyranosyl sorbitol, myoinositol, cyclodextrins, and combinations thereof.

Alternately, or in addition, the pharmaceutical composition further comprises an inorganic salt. In certain embodiments, the inorganic salt is selected from the group consisting of sodium chloride, potassium chloride, magnesium chloride, calcium chloride, sodium phosphate, potassium phosphate, sodium sulfate, or combinations thereof.

Alternately, or in addition, the pharmaceutical composition further comprises an antioxidant. In certain embodiments, the antioxidant is selected from the group consisting of ascorbyl palmitate, butylated hydroxyanisole, butylated hydroxytoluene, potassium metabisulfite, propyl gallate, sodium metabisulfite, sodium thiosulfate, vitamin E, 3,4-dihydroxybenzoic acid, and combinations thereof.

Alternately, or in addition, the pharmaceutical composition further comprises a surfactant. In certain embodiments, the surfactant is selected from the group consisting of polysorbates, sorbitan esters, lipids, phospholipids, phosphatidylethanolamines, fatty acids, fatty acid esters, steroids, EDTA, zinc, and combinations thereof.

Alternately, or in addition, the pharmaceutical composition further comprises a buffer. In certain embodiments, the buffer is selected from the group consisting of citric acid, sodium phosphate, potassium phosphate, acetic acid, ethanolamine, histidine, amino acids, tartaric acid, succinic acid, fumaric acid, lactic acid, tris, HEPES, or combinations thereof.

In some embodiments, the pharmaceutical composition is formulated for parenteral or enteral administration. In some embodiments, the pharmaceutical composition is formulated for intravenous (IV) or subcutaneous administration. In some embodiments, the pharmaceutical composition is in a lyophilized form.

In one aspect, described herein is a liquid or lyophilized composition that comprises a described bifunctional cytokine composition. In some embodiments, the bifunctional cytokine composition is a lyophilized powder. In some embodiments, the lyophilized powder is resuspended in a buffer solution. In some embodiments, the buffer solution comprises a buffer, a sugar, a salt, a surfactant, or any combination thereof. In some embodiments, the buffer solution comprises a phosphate salt. In some embodiments, the phosphate salt is sodium Na₂HPO₄. In some embodiments, the salt is sodium chloride. In some embodiments, the buffer solution comprises phosphate buffered saline. In some embodiments, the buffer solution comprises mannitol. In some embodiments, the lyophilized powder is suspended in a solution comprising about 10 mM Na₂HPO₄ buffer, about 0.022% SDS, and about 50 mg/mL mannitol, and having a pH of about 7.5.

### Dosage Forms

The bifunctional cytokine composition described herein can be in a variety of dosage forms. In some embodiments, the bifunctional cytokine composition is dosed as a lyophilized powder. In some embodiments, the bifunctional cytokine composition is dosed as a suspension. In some embodiments, the bifunctional cytokine composition is dosed as a solution. In some embodiments, the bifunctional cytokine composition is dosed as an injectable solution. In some embodiments, the bifunctional cytokine composition is dosed as an IV solution.

### Methods of Treatment

Methods of treating a cancer in a subject with a bifunctional cytokine composition are contemplated herein. The bifunctional cytokine composition can be any bifunctional cytokine composition described herein (e.g., an IL-2/IL-18 bifunctional cytokine composition).

In some embodiments, the cancer is a solid cancer. In some embodiments, the solid cancer is adrenal cancer, anal cancer, bile duct cancer, bladder cancer, bone cancer, brain cancer, breast cancer, carcinoid cancer, cervical cancer, colorectal cancer, esophageal cancer, eye cancer, gallbladder cancer, gastrointestinal stromal tumor, germ cell cancer, head and neck cancer, kidney cancer, liver cancer, lung cancer, nasal cavity and paranasal sinus cancer, nasopharyngeal cancer, neuroblastoma, neuroendocrine cancer, oral cancer, oropharyngeal cancer, ovarian cancer, pancreatic cancer, pediatric cancer, penile cancer, pituitary cancer, prostate cancer, skin cancer, soft tissue cancer, spinal cord cancer, stomach cancer, testicular cancer, thymus cancer, thyroid cancer, ureteral cancer, uterine cancer, vaginal cancer, or vulvar cancer.

In some embodiments, the cancer is a blood cancer. In some embodiments, the blood cancer is leukemia, non-Hodgkin lymphoma, Hodgkin lymphoma, an AIDS-related lymphoma, multiple myeloma, plasmacytoma, post-transplantation lymphoproliferative disorder, or Waldenstrom macroglobulinemia.

A bifunctional cytokine composition described herein can be administered to a subject in one or more doses. In some embodiments, the bifunctional cytokine composition is administered in a single dose of the effective amount of the bifunctional cytokine composition, including further embodiments in which (i) the bifunctional cytokine composition is administered once a day; or (ii) the bifunctional cytokine composition is administered to the subject multiple times over the span of one day. In some embodiments, the conjugate is administered daily, every other day, twice a week, 3 times a week, once a week, every 2 weeks, every 3 weeks, every 4 weeks, every 5 weeks, every 6 weeks, every 12 weeks, every 3 days, every 4 days, every 5 days, every 6 days, 2 times a week, 3 times a week, 4 times a week, 5 times a week, 6 times a week, once a month, twice a month, 3 times a month, 4 times a month, once every 2 months, once every 3 months, once every 4 months, once every 5 months, or once every 6 months. Administration includes, but is not limited to, injection by any suitable route (e.g., parenteral, enteral, intravenous, subcutaneous, etc.).

An effective response is achieved when the subject experiences partial or total alleviation or reduction of signs or symptoms of illness, and specifically includes, without limitation, prolongation of survival. The expected progression-free survival times may be measured in months to years, depending on prognostic factors including the number of relapses, stage of disease, and other factors. Prolonging survival includes without limitation times of at least 1 month (mo), about at least 2 mos., about at least 3 mos., about at least 4 mos., about at least 6 mos., about at least 1 year, about at least 2 years, about at least 3 years, about at least 4 years, about at least 5 years, etc. Overall or progression-free survival can be also measured in months to years. Alternatively, an effective response may be that a subject's symptoms remain static and do not worsen. Further indications of treatment of indications are described in more detail below. In some instances, a cancer or tumor is reduced by at least 2%, 5%, 10%, 15%,20%,25%,30%,35%,40%,45%,50%,55%,60%,65%,70%,75%, 80%, 85%,90%, 95%, or 100%.

Combination therapies with one or more additional active agents are contemplated herein.

### Definitions

All terms are intended to be understood as they would be understood by a person skilled in the art. Unless defined otherwise, all technical and scientific terms used herein have the same meaning as commonly understood by one of ordinary skill in the art to which the disclosure pertains.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only and is not intended to be limiting.

The terminology used herein is for the purpose of describing particular cases only and is not intended to be limiting. In this application, the use of the singular includes the plural unless specifically stated otherwise. As used herein, the singular forms “a”, “an” and “the” are intended to include the plural forms as well, unless the context clearly indicates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 15%, up to 10%, up to 5%, or up to 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below.

Referred to herein are polymers which are “attached” or “covalently attached” to residues of IL-18 polypeptides. As used herein, “attached” or “covalently attached” means that the polymer is tethered to the indicated reside, and such tethering can include a linking group (i.e., a linker). Thus, for a polymer “attached” or “covalently attached” to a residue, it is expressly contemplated that such linking groups are also encompassed.

As used herein, an “alpha-keto amino acid” or the phrase “alpha-keto” before the name of an amino acid refers to an amino acid or amino acid derivative having a ketone functional group positioned between the carbon bearing the amino group and the carboxylic acid of an amino acid. Alpha-keto amino acids of the instant disclosure have a structure as set forth in the following formula:

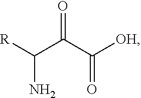

wherein R is the side chain of any natural or unnatural amino acid. The R functionality can be in either the L or D orientation in accordance with standard amino acid nomenclature. In preferred embodiments, alpha-keto amino acids are in the L orientation. When the phrase “alpha-keto” is used before the name of a traditional natural amino acid (e.g., alpha-keto leucine, alpha-keto phenylalanine, etc.) or a common unnatural amino acid (e.g., alpha-keto norleucine, alpha-keto O-methyl-homoserine, etc.), it is intended that the alpha-keto amino acid referred to matches the above formula with the side chain of the referred to amino acid. When an alpha-keto amino acid residue is set forth in a peptide or polypeptide sequence herein, it is intended that a protected version of the relevant alpha-keto amino acid is also encompassed (e.g., for a sequence terminating in a C-terminal alpha-keto amino acid, the terminal carboxylic acid group may be appropriately capped with a protecting group such as a tert-butyl group, or the ketone group with an acetal protecting group). Other protecting groups encompassed are well known in the art.

Binding affinity refers to the strength of a binding interaction between a single molecule and its ligand/binding partner. A higher binding affinity refers to a higher strength bond than a lower binding affinity. In some instances, binding affinity is measured by the dissociation constant (K_{D}) between the two relevant molecules. When comparing K_{D} values, a binding interaction with a lower value will have a higher binding affinity than a binding interaction with a higher value. For a protein-ligand interaction, K_{D} is calculated according to the following formula:

$${K}_{D} = \frac{[ L ] [ P ]}{[ LP ]}$$

where [L] is the concentration of the ligand, [P] is the concentration of the protein, and [LP] is the concentration of the ligand/protein complex.

Referred to herein are certain amino acid sequences (e.g., polypeptide sequences) which have a certain percent sequence identity to a reference sequence or refer to a residue at a position corresponding to a position of a reference sequence. Sequence identity is measured by protein-protein BLAST algorithm using parameters of Matrix BLOSUM62, Gap Costs Existence: 11, Extension: 1, and Compositional Adjustments Conditional Compositional Score Matrix Adjustment. This alignment algorithm is also used to assess if a residue is at a “corresponding” position through an analysis of the alignment of the two sequences being compared.

Unless otherwise specified, is contemplated that “protected” versions of amino acids (e.g., those containing a chemical protecting group affixed to a functionality of the amino acid, particularly a side chain of the amino acid but also at another point of the amino acid) qualify as the same amino acid as the “unprotected” version for sequence identity purposes, particularly for chemically synthesized polypeptides. It is also contemplated that such protected versions are also encompassed by the SEQ ID NOs provided herein. Non-limiting examples of protecting groups which may be encompassed include fluorenylmethyloxycarbonyl (Fmoc), triphenylmethyl (trityl or trt), tert-Butyloxycarbonyl (Boc), 2,2,4,6,7-pentamethyldihydrobenzofuran-5-sulfonyl (Pbf), acetamidomethyl (Acm), tert-butyl (tBu or OtBu), 2,2-dimethyl-1-(4-methoxyphenyl)propane-1,3-diol ketal or acetal, and 2,2-dimethyl-1-(2-nitrophenyl)propane-1,3-diol ketal or acetal. Other protecting groups well known in the art are also encompassed. Similarly, modified versions of natural amino acids are also intended to qualify as natural version of the amino acid for sequence identity purposes. For example, an amino acid comprising a side chain heteroatom which can be covalently modified (e.g., to add a conjugation handle, optionally through a linker), such as a lysine, glutamine, glutamic acid, asparagine, aspartic acid, cysteine, or tyrosine, which has been covalently modified would be counted as the base amino acid (see, e.g., Structure 2 below, which would be counted as a lysine for sequence identity and SEQ ID purposes). Similarly, an amino acid comprising another group added to the C or N-terminus would be counted as the base amino acid.

The term “pharmaceutically acceptable” refers to approved or approvable by a regulatory agency of the federal or a state government or listed in the U.S. Pharmacopeia (U.S.P.) or other generally recognized pharmacopeia for use in animals, including humans.

A “pharmaceutically acceptable excipient, carrier, or diluent” refers to an excipient, carrier, or diluent that can be administered to a subject, together with an agent, and which does not destroy the pharmacological activity thereof and is nontoxic when administered in doses sufficient to deliver a therapeutic amount of the agent.

A “pharmaceutically acceptable salt” suitable for the disclosure may be an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethyl sulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH₂)n-COOH where n is 0-4, and the like. Similarly, pharmaceutically acceptable cations include, but are not limited to sodium, potassium, calcium, aluminum, lithium and ammonium. Those of ordinary skill in the art will recognize from this disclosure and the knowledge in the art that further pharmaceutically acceptable salts include those listed by Remington's Pharmaceutical Sciences, 17th ed., Mack Publishing Company, Easton, PA, p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The term “subject” refers to an animal which is the object of treatment, observation, or experiment. By way of example only, a subject includes, but is not limited to, a mammal, including, but not limited to, a human or a non-human mammal, such as a non-human primate, bovine, equine, canine, ovine, or feline.

Certain formulas and other illustrations provided herein depict triazole reaction products resulting from azide-alkyne cycloaddition reactions. While such formulas generally depict only a single regioisomer of the resulting triazole formed in the reaction, it is intended that the formulas encompass both resulting regioisomers. Thus, while the formulas depict only a single regioisomer (e.g.

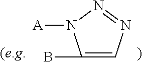

it is intended that the other regioisomer

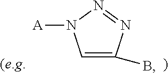

is also encompassed.

The term “optional” or “optionally” denotes that a subsequently described event or circumstance can but need not occur, and that the description includes instances where the event or circumstance occurs and instances in which it does not.

The term “moiety” refers to a specific segment or functional group of a molecule. Chemical moieties are often recognized chemical entities embedded in or appended to a molecule.

As used herein, the term “number average molecular weight” (Mn) means the statistical average molecular weight of all the individual units in a sample, and is defined by Formula (1):

$$Mn = \frac{\sum {N}_{i} {M}_{i}}{\sum {N}_{i}} \qquad Formula ( 1 )$$

where Mᵢ is the molecular weight of a unit and Nᵢ is the number of units of that molecular weight.

As used herein, the term “weight average molecular weight” (Mw) means the number defined by Formula (2):

$$Mw = \frac{\sum {N}_{i} {M}_{i}^{2}}{\sum {N}_{i} {M}_{i}} \qquad Formula ( 2 )$$

where Mᵢ is the molecular weight of a unit and Nᵢ is the number of units of that molecular weight.

As used herein, “peak molecular weight” (Mp) means the molecular weight of the highest peak in a given analytical method (e.g., mass spectrometry, size exclusion chromatography, dynamic light scattering, analytical centrifugation, etc.).

As used herein, “conjugation handle” refers to a reactive group capable of forming a bond upon contacting a complementary reactive group. In some instances, a conjugation handle preferably does not have a substantial reactivity with other molecules which do not comprise the intended complementary reactive group. Non-limiting examples of conjugation handles, their respective complementary conjugation handles, and corresponding reaction products can be found in the table below. While table headings place certain reactive groups under the title “conjugation handle” or “complementary conjugation handle,” it is intended that any reference to a conjugation handle can instead encompass the complementary conjugation handles listed in the table (e.g., a trans-cyclooctene can be a conjugation handle, in which case tetrazine would be the complementary conjugation handle). In some instances, amine conjugation handles and conjugation handles complementary to amines are less preferable for use in biological systems owing to the ubiquitous presence of amines in biological systems and the increased likelihood for off-target conjugation.

Table of Conjugation Handles

| | | Reaction |
|---|---|---|
| Conjugation Handle | Complementary Conjugation Handle | Product |
| Sulfhydryl | alpha-halo-carbonyl (e.g., bromoacetamide), alpha- | thioether |
| | beta unsaturated carbonyl (e.g., maleimide, | |
| | acrylamide) | |
| Azide | alkyne (e.g., terminal alkyne, substituted cyclooctyne | triazole |
| | (e.g., dibenzocycloocytne (DBCO), | |
| | difluorocyclooctyne, bicyclo[6.1.0]nonyne, etc.)) | |
| Phosphine | Azide/ester pair | amide |
| Tetrazine | trans-cyoclooctene | dihydropyridazine |
| Amine | Activated ester (e.g., N-hydroxysuccinimide ester, | amide |
| | pentaflurophenyl ester) | |
| isocyanate | amine | urea |
| epoxide | amine | alkyl-amine |
| hydroxyl amine | aldehyde, ketone | oxime |
| hydrazide | aldehyde, ketone | hydrazone |
| potassium acyl | O-substituted hydroxylamine (e.g., O- | amide |
| trifluoroborate | carbamoylhydroxylamine) | |

Throughout the instant application, prefixes are used before the term “conjugation handle” to denote the functionality to which the conjugation handle is linked. For example, a “protein conjugation handle” is a conjugation handle attached to a protein (either directly or through a linker), an “antibody conjugation handle” is a conjugation handle attached to an antibody (either directly or through a linker), and a “linker conjugation handle” is a conjugation handle attached to a linker group (e.g., a bifunctional linker used to link a synthetic protein and an antibody).

The term “alkyl” refers to a straight or branched hydrocarbon chain radical, having from one to twenty carbon atoms, and which is attached to the rest of the molecule by a single bond. An alkyl comprising up to 10 carbon atoms is referred to as a C₁-C₁₀ alkyl, likewise, for example, an alkyl comprising up to 6 carbon atoms is a C₁-C₆ alkyl. Alkyls (and other moieties defined herein) comprising other numbers of carbon atoms are represented similarly. Alkyl groups include, but are not limited to, C₁-C₁₀ alkyl, C₁-C₉ alkyl, C₁-C₈ alkyl, C₁-C₇ alkyl, C₁-C₆ alkyl, C₁-C₅ alkyl, C₁-C₄ alkyl, C₁-C₃ alkyl, C₁-C₂ alkyl, C₂-C₈ alkyl, C₃-C₈ alkyl and C₄-C₈ alkyl. Representative alkyl groups include, but are not limited to, methyl, ethyl, n-propyl, 1-methyl ethyl (i-propyl), n-butyl, i-butyl, s-butyl, n-pentyl, 1,1-dimethyl ethyl (t-butyl), 3-methylhexyl, 2-methylhexyl, 1-ethyl-propyl, and the like. In some embodiments, the alkyl is methyl or ethyl. In some embodiments, the alkyl is —CH(CH₃)₂ or —C(CH₃)₃. Unless stated otherwise specifically in the specification, an alkyl group may be optionally substituted. “Alkylene” or “alkylene chain” refers to a straight or branched divalent hydrocarbon chain linking the rest of the molecule to a radical group. In some embodiments, the alkylene is —CFF—, —CH₂CH₂—, or —CH₂CH₂CH₂—. In some embodiments, the alkylene is —CH₂—. In some embodiments, the alkylene is —CH₂CH₂—. In some embodiments, the alkylene is —CH₂CH₂CH₂—. Unless stated otherwise specifically in the specification, an alkylene group may be optionally substituted.

The term “alkenylene” or “alkenylene chain” refers to a straight or branched divalent hydrocarbon chain in which at least one carbon-carbon double bond is present linking the rest of the molecule to a radical group. In some embodiments, the alkenylene is —CH═CH—, —CH₂CH═CH—, or —CH═CHCH₂—. In some embodiments, the alkenylene is —CH═CH—. In some embodiments, the alkenylene is —CH₂CH═CH—. In some embodiments, the alkenylene is —CH═CHCH₂—.

The term “alkynyl” refers to a type of alkyl group in which at least one carbon-carbon triple bond is present. In one embodiment, an alkenyl group has the formula —C^{∘}C—R^{X}, wherein R^{x} refers to the remaining portions of the alkynyl group. In some embodiments, R^{x} is H or an alkyl. In some embodiments, an alkynyl is selected from ethynyl, propynyl, butynyl, pentynyl, hexynyl, and the like.

The term “alkynyl” refers to a type of alkyl group in which at least one carbon-carbon triple bond is present. In one embodiment, an alkenyl group has the formula —C^{∘}C—R^{X}, wherein R^{x} refers to the remaining portions of the alkynyl group. In some embodiments, R^{x} is H or an alkyl. In some embodiments, an alkynyl is selected from ethynyl, propynyl, butynyl, pentynyl, hexynyl, and the like.

The term “aryl” refers to a radical comprising at least one aromatic ring wherein each of the atoms forming the ring is a carbon atom. Aryl groups can be optionally substituted. Examples of aryl groups include, but are not limited to phenyl, and naphthyl. In some embodiments, the aryl is phenyl. Depending on the structure, an aryl group can be a monoradical or a diradical (i.e., an arylene group). Unless stated otherwise specifically in the specification, the term “aryl” or the prefix “ar-” (such as in “aralkyl”) is meant to include aryl radicals that are optionally substituted. In some embodiments, an aryl group comprises a partially reduced cycloalkyl group defined herein (e.g., 1,2-dihydronaphthalene). In some embodiments, an aryl group comprises a fully reduced cycloalkyl group defined herein (e.g., 1,2,3,4-tetrahydronaphthalene). When aryl comprises a cycloalkyl group, the aryl is bonded to the rest of the molecule through an aromatic ring carbon atom. An aryl radical can be a monocyclic or polycyclic (e.g., bicyclic, tricyclic, or tetracyclic) ring system, which may include fused, spiro or bridged ring systems.

The term “cycloalkyl” refers to a monocyclic or polycyclic non-aromatic radical, wherein each of the atoms forming the ring (i.e. skeletal atoms) is a carbon atom. In some embodiments, cycloalkyls are saturated or partially unsaturated. In some embodiments, cycloalkyls are spirocyclic or bridged compounds. In some embodiments, cycloalkyls are fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloalkyls having from three to ten carbon atoms, from three to eight carbon atoms, from three to six carbon atoms, or from three to five carbon atoms. Monocyclic cycloalkyl radicals include, for example, cyclopropyl, cyclobutyl, cyclopentyl, cyclohexyl, cycloheptyl, and cyclooctyl. In some embodiments, the monocyclic cycloalkyl is cyclopentyl. In some embodiments, the monocyclic cycloalkyl is cyclopentenyl or cyclohexenyl. In some embodiments, the monocyclic cycloalkyl is cyclopentenyl. Polycyclic radicals include, for example, adamantyl, 1,2-dihydronaphthalenyl, 1,4-dihydronaphthalenyl, tetrainyl, decalinyl, 3,4-dihydronaphthalenyl-1(2H)-one, spiro[2.2]pentyl, norbornyl and bicycle[1.1.1]pentyl. Unless otherwise stated specifically in the specification, a cycloalkyl group may be optionally substituted.

The term “heteroalkylene” or “heteroalkylene chain” refers to a straight or branched divalent heteroalkyl chain linking the rest of the molecule to a radical group. Unless stated otherwise specifically in the specification, the heteroalkyl or heteroalkylene group may be optionally substituted as described below. Representative heteroalkylene groups include, but are not limited to —CH₂—O—CH₂—, —CH₂—N(alkyl)-CH₂—, —CH₂—N(aryl)-CH₂—, —OCH₂CH₂O—, —OCH₂CH₂OCH₂CH₂O—, or —O CH₂CH₂ O CH₂CH₂ O CH₂CH₂ O—.

The term “heteocycloalkyl” refers to a cycloalkyl group that includes at least one heteroatom selected from nitrogen, oxygen, and sulfur. Unless stated otherwise specifically in the specification, the heterocycloalkyl radical may be a monocyclic, or bicyclic ring system, which may include fused (when fused with an aryl or a heteroaryl ring, the heterocycloalkyl is bonded through a non-aromatic ring atom) or bridged ring systems. The nitrogen, carbon or sulfur atoms in the heterocyclyl radical may be optionally oxidized. The nitrogen atom may be optionally quaternized. The heterocycloalkyl radical is partially or fully saturated. Examples of heterocycloalkyl radicals include, but are not limited to, dioxolanyl, thienyl[1,3]dithianyl, tetrahydroquinolyl, tetrahydroisoquinolyl, decahydroquinolyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl. The term heterocycloalkyl also includes all ring forms of carbohydrates, including but not limited to monosaccharides, disaccharides and oligosaccharides. Unless otherwise noted, heterocycloalkyls have from 2 to 12 carbons in the ring. In some embodiments, heterocycloalkyls have from 2 to 10 carbons in the ring. In some embodiments, heterocycloalkyls have from 2 to 10 carbons in the ring and 1 or 2 N atoms. In some embodiments, heterocycloalkyls have from 2 to 10 carbons in the ring and 3 or 4 N atoms. In some embodiments, heterocycloalkyls have from 2 to 12 carbons, 0-2 N atoms, 0-2 O atoms, 0-2 P atoms, and 0-1 S atoms in the ring. In some embodiments, heterocycloalkyls have from 2 to 12 carbons, 1-3 N atoms, 0-1 O atoms, and 0-1 S atoms in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl group may be optionally substituted.

The term “heteroaryl” refers to an aryl group that includes one or more ring heteroatoms selected from nitrogen, oxygen, and sulfur. In some embodiments, heteroaryl is monocyclic or bicyclic. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, furazanyl, indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Illustrative examples of bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, heteroaryl is pyridinyl, pyrazinyl, pyrimidinyl, thiazolyl, thienyl, thiadiazolyl or furyl. In some embodiments, a heteroaryl contains 0-6 N atoms in the ring. In some embodiments, a heteroaryl contains 1-4 N atoms in the ring. In some embodiments, a heteroaryl contains 4-6 N atoms in the ring. In some embodiments, a heteroaryl contains 0-4 N atoms, 0-1 O atoms, 0-1 P atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉ heteroaryl. In some embodiments, monocyclic heteroaryl is a C₁-C₅ heteroaryl. In some embodiments, monocyclic heteroaryl is a 5-membered or 6-membered heteroaryl. In some embodiments, a bicyclic heteroaryl is a C6-C9 heteroaryl. In some embodiments, a heteroaryl group comprises a partially reduced cycloalkyl or heterocycloalkyl group defined herein (e.g., 7,8-dihydroquinoline). In some embodiments, a heteroaryl group comprises a fully reduced cycloalkyl or heterocycloalkyl group defined herein (e.g., 5,6,7, 8-tetrahydroquinoline). When heteroaryl comprises a cycloalkyl or heterocycloalkyl group, the heteroaryl is bonded to the rest of the molecule through a heteroaromatic ring carbon or hetero atom. A heteroaryl radical can be a monocyclic or polycyclic (e.g., bicyclic, tricyclic, or tetracyclic) ring system, which may include fused, spiro or bridged ring systems.

The term “optionally substituted” or “substituted” means that the referenced group is optionally substituted with one or more additional group(s) individually and independently selected from D, halogen, —CN, —NH2, —NH(alkyl), —N(alkyl)2, —OH, —CO2H, —C0₂alkyl, —C(=0)NH₂, —C(=0)NH(alkyl), —C(=0)N(alkyl)₂, —S(=0)₂NH₂, —S(=0)₂NH(alkyl), —S(=0)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from D, halogen, —CN, —NH₂, —NH(CH₃), —N(CH₃)₂, —OH, —C0₂H, —C0₂(Ci-C₄alkyl), —C(=0)NH₂, —C(=0)NH(C 1-C₄alkyl), —C(=0)N(Ci-C₄alkyl)₂, —S(=0)₂NH₂, —S(=0)₂NH(Ci-C₄alkyl), —S(=0)₂N(Ci-C₄alkyl)₂, Ci-C₄alkyl, C₃-C₆cycloalkyl, Ci-C₄fluoroalkyl, Ci-C₄heteroalkyl, Ci-C₄alkoxy, Ci-C₄fluoroalkoxy, —SCi-C₄alkyl, —S(=0)Ci-C₄alkyl, and —S(=0)₂Ci-C₄alkyl. In some embodiments, optional substituents are independently selected from D, halogen, —CN, —NH₂, —OH, —NH(CH₃), —N(CH₃)₂, —NH(cyclopropyl), —CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic) includes oxo (=0).

### Sequences (SEQ ID NOS) of Exemplary Cytokines

TABLE 4
Exemplary IL-18 Sequences

SEQ ID

| | | |
|---|---|---|
| NO: | Modification | Sequence |

| | | |
|---|---|---|
| 1 | Native | YFGKLESKLS VIRNLNDQVL FIDQGNRPLF EDMTDSDCRD |
| | sequence | NAPRTIFIIS MYKDSQPRGM AVTISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 2 | E6K, C38A, | YFGKLKSKLS VIRNLNDQVL FIDQGNRPLF |
| | K53A, C68A, | EDMTDSDARD NAPRTIFIIS MYADSQPRGM |
| | E85C | AVTISVKAEK ISTLSCENKI ISFKCMNPPD |
| | | NIKDTKSDII FFQRSVPGHD NKMQFESSSY |
| | | EGYFLACEKE RDLFKLILKK EDELGDRSIM |
| | | FTVQNED |
| 3 | E6K, V11I, | YFGKLKSKLS IIRNLNDQVL FIDQGNRPLF |
| | C38A, K53A, | EDMTDSDARD NAPRTIFIIS MYADSQPRGM |
| | T63A, C68A, | AVAISVKAEK ISTLSAENKI ISFKCMNPPD |
| | C76A, E85C, | NIKDTKSDII FFQRSVPGHD NKMQFESSSY |
| | C127A | EGYFLAAEKE RDLFKLILKK EDELGDRSIM |
| | | FTVQNED |
| 4 | E6K, C38A, | YFGKLKSKLS VIRNLNDQVL FIDQGNRPLF |
| | K53A, C68A, | EDMTDSDARD NAPRTIFIIS MYADSQPRGM |
| | M86C | AVTISVKAEK ISTLSCENKI ISFKECNPPD |
| | | NIKDTKSDII FFQRSVPGHD NKMQFESSSY |
| | | EGYFLACEKE RDLFKLILKK EDELGDRSIM |
| | | FTVQNED |

| | | |
|---|---|---|
| 5 | E6K, V11I, | YFGKLKSKLS IIRNLNDQVL FIDQGNRPLF EDMTDSDARD |
| | C38A, K53A, | NAPRTIFIIS MYADSQPRGM AVAISVKAEK ISTLSAENKI |
| | T63A, C68A, | ISFKECNPPD NIKDTKSDII FFQRSVPGHD NKMQFESSSY |
| | C76A, M86C, | EGYFLAAEKE RDLFKLILKK EDELGDRSIM FTVQNED |
| | C127A | |
| 6 | E6K, V11I, | YFGKLKSKLS IIRNLNDQVL FIDQGNRPLF |
| | C38A, K53A, | EDMTDSDARD NAPRTIFIIS MYADSQPRGM |
| | T63A, C68A, | AVAISVKAEK ISTLSAENKI ISFKEMNPPD |
| | C76A, D98C, | NIKDTKSCII FFQRSVPGHD NKMQFESSSY |
| | C127A | EGYFLAAEKE RDLFKLILKK EDELGDRSIM |
| | | FTVQNED |
| 7 | E6K, C38A, | YFGKLKSKLS VIRNLNDQVL FIDQGNRPLF EDMTDSDARD |
| | K53A, C68A, | NAPRTIFIIS MYADSQPRGM AVTISVKAEK ISTLSAENKI |
| | C76A, M86C, | ISFKECNPPD NIKDTKSDII FFQRSVPGHD NKMQFESSSY |
| | C127A | EGYFLAAEKE RDLFKLILKK EDELGDRSIM FTVQNED |
| 8 | E6K, V11I, | YFGKLKSKLS IIRNLNDQVL FIDQGNRPLF EDMTDSDARD |
| | C38A, K53A, | NAPRTIFIIS MYADSQPRGM AVAISVKAEK ISTLSAENKI |
| | T63A, C68A, | ISFKEMNPPD NIKDCKSDII FFQRSVPGHD NKMQFESSSY |
| | C76A, T95C, | EGYFLAAEKE RDLFKLILKK EDELGDRSIM FTVQNED |
| | C127A | |
| 9 | E6K, C38A, | YFGKLKSKLS VIRNLNDQVL FIDQGNRPLF |
| | K53A, C68A, | EDMTDSDARD NAPRTIFIIS MYADSQPRGM |
| | D98C | AVTISVKAEK ISTLSCENKI ISFKEMNPPD |
| | | NIKDTKSCII FFQRSVPGHD NKMQFESSSY |
| | | EGYFLACEKE RDLFKLILKK EDELGDRSIM |
| | | FTVQNED |
| 10 | E6K, C38A, | YFGKLKSKLS VIRNLNDQVL FIDQGNRPLF EDMTDSDARD |
| | K53A, C68A, | NAPRTIFIIS MYADSQPRGM AVTISVKAEK ISTLSAENKI |
| | C76A, D98C, | ISFKEMNPPD NIKDTKSCII FFQRSVPGHD NKMQFESSSY |
| | C127A | EGYFLAAEKE RDLFKLILKK EDELGDRSIM FTVQNED |
| 11* | E6K, V11I, | YFGKLKSKLS IIRNLNDQVL FIDQGNRPLF EDMTDSCARD |
| | C38A, K53A, | NAPRTIFIIS MYADSQPRGM AVTISVKCEK ISTLSAENKI |
| | C76A, C127A | ISFKEMNPPD NIKDTKSDII FFQRSVPGHD NKMQFESSSY |
| | | EGYFLAAEKE RDLFKLILKK EDELGDRSIM FTVQNED |
| 12* | E6K, C38A, | YFGKLKSKLS VIRNLNDQVL FIDQGNRPLF EDMTDSDARD |
| | K53A, T63A, | NAPRTIFIIS MYADSQPRGM AVAISVKCEK ISTLSAENKI |
| | C76A, C127A | ISFKEMNPPD NIKDTKSDII FFQRSVPGHD NKMQFESSSY |
| | | EGYFLAAEKE RDLFKLILKK EDELGDRSIM FTVQNED |
| 13 | E6K, K53A, | YFGKLKSKLS VIRNLNDQVL FIDQGNRPLF |
| | T63N | EDMTDSDCRD NAPRTIFIIS MYADSQPRGM |
| | | AVNISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 14 | E6K, K53A, | YFGKLKSKLS VIRNLNDQVL FIDQGNRPLF |
| | S50A, T63N | EDMTDSDCRD NAPRTIFIIA MYADSQPRGM |
| | | AVNISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 15 | E6K, K53A, | YFGKLKSKLS VIRNLNDQVL FIDQGNRPLF |
| | S50H, T63N | EDMTDSDCRD NAPRTIFIIH MYADSQPRGM |
| | | AVNISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 16 | E6K, K53A, | YFGKLKSKLS VIRNLNDQVL FIDQGNRPLF |
| | T63N, S65A | EDMTDSDCRD NAPRTIFIIS MYADSQPRGM |
| | | AVNIAVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 17 | E6K, K53A, | YFGKLKSKLS VIRNLNDQVL FIDQGNRPLF |
| | S50H | EDMTDSDCRD NAPRTIFIIH MYADSQPRGM |
| | | AVTISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 18 | E6K, C38A, | YFGKLKSKLS VIRNLNDQVL FIDQGNRPLF |
| | K53A, C68A | EDMTDSDARD NAPRTIFIIS MYADSQPRGM |
| | | AVTISVKAEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 19 | E6K, K53A, | YFGKLKSKLS VIRNLNDQVL FIDQGNRPLF EDMTDSDCRD |
| | K79A | NAPRTIFIIS MYADSQPRGM AVTISVKCEK ISTLSCENAI |
| | | ISFKEMNPPD NIKDTKSDII FFQRSVPGHD NKMQFESSSY |
| | | EGYFLACEKE RDLFKLILKK EDELGDRSIM FTVQNED |
| 20 | E6K, K53A, | YFGKLKSKLS VIRNLNDQVL FIDQGNRPLF EDMTDSDCRD |
| | R104A | NAPRTIFIIS MYADSQPRGM AVTISVKCEK ISTLSCENAI |
| | | ISFKEMNPPD NIKDTKSDII FFQRSVPGHD NKMQFESSSY |
| | | EGYFLACEKE RDLFKLILKK EDELGDRSIM FTVQNED |
| 21 | E6K, K53A, | YFGKLKSKLS VIRNLNDQVL FIDQGNRPLF EDMTDSDCRD |
| | G108A | NAPRTIFIIS MYADSQPRGM AVTISVKCEK ISTLSCENKI |
| | | ISFKEMNPPD NIKDTKSDII FFQRSVPAHD NKMQFESSSY |
| | | EGYFLACEKE RDLFKLILKK EDELGDRSIM FTVQNED |
| 22 | E6K, K53A, | YFGKLKSKLS VIRNLNDQVL FIDQGNRPLF EDMTDSDCRD |
| | H109A | NAPRTIFIIS MYADSQPRGM AVTISVKCEK ISTLSCENKI |
| | | ISFKEMNPPD NIKDTKSDII FFQRSVPGAD NKMQFESSSY |
| | | EGYFLACEKE RDLFKLILKK EDELGDRSIM FTVQNED |
| 23 | E6K, K53A, | YFGKLKSKLS VIRNLNDQVL FIDQGNRPLF EDMTDSDCRD |
| | K112A | NAPRTIFIIS MYADSQPRGM AVTISVKCEK ISTLSCENKI |
| | | ISFKEMNPPD NIKDTKSDII FFQRSVPGHD NAMQFESSSY |
| | | EGYFLACEKE RDLFKLILKK EDELGDRSIM FTVQNED |
| 24 | E6K, C38A, | YFGKLKSKLS VIRNLNDQVL FIDQGNRPLF |
| | K53A, T63A, | EDMTDSDARD NAPRTIFIIS MYADSQPRGM |
| | C76A | AVAISVKCEK ISTLSAENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 25 | E6K, C38Q, | YFGKLKSKLS VIRNLNDQVL FIDQGNRPLF |
| | K53A, T63A, | EDMTDSDQRD NAPRTIFIIS MYADSQPRGM |
| | C76A | AVAISVKCEK ISTLSAENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 26 | E6K, C38A, | YFGKLKSKLS VIRNLNDQVL FIDQGNRPLF |
| | K53A, T63A, | EDMTDSDARD NAPRTIFIIS MYADSQPRGM |
| | C127A | AVAISVKCEK ISTLSCENKI ISFKEMNPPD |
| | | NIKDTKSDII FFQRSVPGHD NKMQFESSSY |
| | | EGYFLAAEKE RDLFKLILKK EDELGDRSIM |
| | | FTVQNED |
| 27 | E6K, C38Q, | YFGKLKSKLS VIRNLNDQVL FIDQGNRPLF |
| | K53A, T63A, | EDMTDSDQRD NAPRTIFIIS MYADSQPRGM |
| | C127A | AVAISVKCEK ISTLSCENKI ISFKEMNPPD |
| | | NIKDTKSDII FFQRSVPGHD NKMQFESSSY |
| | | EGYFLAAEKE RDLFKLILKK EDELGDRSIM |
| | | FTVQNED |
| 28 | E6K, C38A, | YFGKLKSKLS VIRNLNDQVL FIDQGNRPLF |
| | K53A, T63A, | EDMTDSDARD NAPRTIFIIS MYADSQPRGM |
| | C76A, C127A | AVAISVKCEK ISTLSAENKI ISFKEMNPPD |
| | | NIKDTKSDII FFQRSVPGHD NKMQFESSSY |
| | | EGYFLAAEKE RDLFKLILKK EDELGDRSIM |
| | | FTVQNED |
| 29 | E6K, V11I, | YFGKLKSKLS IIRNLNDQVL FIDQGNRPLF |
| | C38A, K53A, | EDMTDSDARD NAPRTIFIIS MYADSQPRGM |
| | T63A | AVAISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 30 | E6K, V11I, | YFGKLKSKLS IIRNLNDQVL FIDQGNRPLF |
| | C38A, K53A, | EDMTDSDARD NAPRTIFIIS MYADSQPRGM |
| | T63A, C76A, | AVAISVKCEK ISTLSAENKI ISFKEMNPPD |
| | C127A | NIKDTKSDII FFQRSVPGHD NKMQFESSSY |
| | | EGYFLAAEKE RDLFKLILKK EDELGDRSIM |
| | | FTVQNED |
| 31 | C38A, C76A, | YFGKLESKLS VIRNLNDQVL FIDQGNRPLF |
| | C127A | EDMTDSDARD NAPRTIFIIS MYKDSQPRGM |
| | | AVTISVKCEK ISTLSAENKI ISFKEMNPPD |
| | | NIKDTKSDII FFQRSVPGHD NKMQFESSSY |
| | | EGYFLAAEKE RDLFKLILKK EDELGDRSIM |
| | | FTVQNED |
| 32 | C38A | YFGKLESKLS VIRNLNDQVL FIDQGNRPLF |
| | | EDMTDSDARD NAPRTIFIIS MYKDSQPRGM |
| | | AVTISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 33 | E6K, C38A, | YFGKLKSKLS VIRNLNDQVL FIDQGNRPLF |
| | K53A, T63A | EDMTDSDARD NAPRTIFIIS MYADSQPRGM |
| | | AVAISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 34 | E06K, K53A, | YFGKLKSKLS VIRNLNDQVL FIDQGNRPLF |
| | S55A | EDMTDSDCRD NAPRTIFIIS MYADAQPRGM AVTISVKCEK |
| | | ISTLSCENKI ISFKEMNPPD NIKDTKSDII FFQRSVPGHD |
| | | NKMQFESSSY EGYFLACEKE RDLFKLILKK EDELGDRSIM |
| | | FTVQNED |
| 35 | Y01G, F02A, | GAGKLKSKLS VIRNLNDQVL FIDQGNRPLF |
| | E06K, M51G, | EDMTDSDCRD NAPRTIFIIS GYAAAQPRGM AVAISVKCEK |
| | K53A, D54A, | ISTLSCENKI ISFKEMNPPD NIKDTKSDII FFQRSVPGHD |
| | S55A, T63A | NKMQFESSSY EGYFLACEKE RDLFKLILKK EDELGDRSIM |
| | | FTVQNED |
| 36 | K53A | YFGKLESKLS VIRNLNDQVL FIDQGNRPLF EDMTDSDCRD |
| | | NAPRTIFIIS MYADSQPRGM AVTISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 37 | S55A | YFGKLESKLS VIRNLNDQVL FIDQGNRPLF EDMTDSDCRD |
| | | NAPRTIFIIS MYKDAQPRGM AVTISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 38 | E06K | YFGKLKSKLS VIRNLNDQVL FIDQGNRPLF |
| | | EDMTDSDCRD NAPRTIFIIS MYKDSQPRGM AVTISVKCEK |
| | | ISTLSCENKI ISFKEMNPPD NIKDTKSDII FFQRSVPGHD |
| | | NKMQFESSSY EGYFLACEKE RDLFKLILKK EDELGDRSIM |
| | | FTVQNED |
| 39 | E06K, K53A | YFGKLKSKLS VIRNLNDQVL FIDQGNRPLF |
| | | EDMTDSDCRD NAPRTIFIIS MYADSQPRGM AVTISVKCEK |
| | | ISTLSCENKI ISFKEMNPPD NIKDTKSDII FFQRSVPGHD |
| | | NKMQFESSSY EGYFLACEKE RDLFKLILKK EDELGDRSIM |
| | | FTVQNED |
| 40 | E06K, S55A | YFGKLKSKLS VIRNLNDQVL FIDQGNRPLF |
| | | EDMTDSDCRD NAPRTIFIIS MYKDAQPRGM AVTISVKCEK |
| | | ISTLSCENKI ISFKEMNPPD NIKDTKSDII FFQRSVPGHD |
| | | NKMQFESSSY EGYFLACEKE RDLFKLILKK EDELGDRSIM |
| | | FTVQNED |
| 41 | K53A, S55A | YFGKLESKLS VIRNLNDQVL FIDQGNRPLF EDMTDSDCRD |
| | | NAPRTIFIIS MYADAQPRGM AVTISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 42 | E06K, K53A, | YFGKLKSKLS VIRNLNDQVL FIDQGNRPLF |
| | S55A, T63A | EDMTDSDCRD NAPRTIFIIS MYADAQPRGM |
| | | AVAISVKCEK ISTLSCENKI ISFKEMNPPD NIKDTKSDII |
| | | FFQRSVPGHD NKMQFESSSY EGYFLACEKE RDLFKLILKK |
| | | EDELGDRSIM FTVQNED |
| 43 | E06K, K53A, | GFGKLKSKLS VIRNLNDQVL FIDQGNRPLF |
| | S55A, Y01G | EDMTDSDCRD NAPRTIFIIS MYADAQPRGM AVTISVKCEK |
| | | ISTLSCENKI ISFKEMNPPD NIKDTKSDII FFQRSVPGHD |
| | | NKMQFESSSY EGYFLACEKE RDLFKLILKK EDELGDRSIM |
| | | FTVQNED |
| 44 | E06K, K53A, | YAGKLKSKLS VIRNLNDQVL FIDQGNRPLF |
| | S55A, F02A | EDMTDSDCRD NAPRTIFIIS MYADAQPRGM AVTISVKCEK |
| | | ISTLSCENKI ISFKEMNPPD NIKDTKSDII FFQRSVPGHD |
| | | NKMQFESSSY EGYFLACEKE RDLFKLILKK EDELGDRSIM |
| | | FTVQNED |
| 45 | E06K, K53A, | YFGKLKSKLS VIRNLNDQVL FIDQGNRPLF |
| | S55A, D54A | EDMTDSDCRD NAPRTIFIIS MYAAAQPRGM AVTISVKCEK |
| | | ISTLSCENKI ISFKEMNPPD NIKDTKSDII FFQRSVPGHD |
| | | NKMQFESSSY EGYFLACEKE RDLFKLILKK EDELGDRSIM |
| | | FTVQNED |
| 46 | E06K, K53A, | YFGKLKSKLS VIRNLNDQVL FIDQGNRPLF |
| | S55A, M51G | EDMTDSDCRD NAPRTIFIIS GYADAQPRGM AVTISVKCEK |
| | | ISTLSCENKI ISFKEMNPPD NIKDTKSDII FFQRSVPGHD |
| | | NKMQFESSSY EGYFLACEKE RDLFKLILKK EDELGDRSIM |
| | | FTVQNED |
| 47 | C38S, C68S, | YFGKLESKLS VIRNLNDQVL FIDQGNRPLF EDMTDSDSRD |
| | C76S, C127S | NAPRTIFIIS MYKDSQPRGM AVTISVKSEK ISTLSSENKI |
| | | ISFKEMNPPD NIKDTKSDII FFQRSVPGHD NKMQFESSSY |
| | | EGYFLASEKE RDLFKLILKK EDELGDRSIM FTVQNED |
| 48 | C38S, C68S, | YFGKLESKLS VIRNLNDQVL FIDQGNRPLF EDMTDSDSRD |
| | C76S, C127S, | NAPRTIFIIS MYKDSQPRGM AVTISVKSEC ISTLSSENKI |
| | K70C | ISFKEMNPPD NIKDTKSDII FFQRSVPGHD NKMQFESSSY |
| | | EGYFLASEKE RDLFKLILKK EDELGDRSIM FTVQNED |
| 49 | E06K, K53A, | YFGKLKSKLS VIRNLNDQVL FIDQGNRPLF EDMTDSDSRD |
| | S55A, C38S, | NAPRTIFIIS MYADAQPRGM AVTISVKSEC ISTLSSENKI |
| | C68S, C76S, | ISFKEMNPPD NIKDTKSDII FFQRSVPGHD NKMQFESSSY |
| | C127S, K70C | EGYFLASEKE RDLFKLILKK EDELGDRSIM FTVQNED |
| 50 | E06K, K53A, | YFGKLKSKLS VIRNLNDQVL FIDQGNRPLF |
| | T63A | EDMTDSDCRD NAPRTIFIIS MYADSQPRGM AVAISVKCEK |
| | | ISTLSCENKI ISFKEMNPPD NIKDTKSDII FFQRSVPGHD |
| | | NKMQFESSSY EGYFLACEKE RDLFKLILKK EDELGDRSIM |
| | | FTVQNED |
| 51 | T63A | YFGKLESKLS VIRNLNDQVL FIDQGNRPLF EDMTDSDCRD |
| | | NAPRTIFIIS MYKDSQPRGM AVAISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 52 | E06K, T63A | YFGKLKSKLS VIRNLNDQVL FIDQGNRPLF |
| | | EDMTDSDCRD NAPRTIFIIS MYKDSQPRGM AVAISVKCEK |
| | | ISTLSCENKI ISFKEMNPPD NIKDTKSDII FFQRSVPGHD |
| | | NKMQFESSSY EGYFLACEKE RDLFKLILKK EDELGDRSIM |
| | | FTVQNED |
| 53 | K53A, T63A | YFGKLESKLS VIRNLNDQVL FIDQGNRPLF EDMTDSDCRD |
| | | NAPRTIFIIS MYADSQPRGM AVAISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 54 | E06K, K53A, | YFGKLKSKLS VIRNLNDQVL FIDQGNRPLF EDMTDSDSRD |
| | C38S, C68S, | NAPRTIFIIS MYADSQPRGM AVTISVKSEC ISTLSSENKI |
| | C76S, C127S, | ISFKEMNPPD NIKDTKSDII FFQRSVPGHD NKMQFESSSY |
| | K70C | EGYFLASEKE RDLFKLILKK EDELGDRSIM FTVQNED |
| 55 | K53A, T63A, | YFGKLESKLS VIRNLNDQVL FIDQGNRPLF EDMTDSDSRD |
| | C38S, C68S, | NAPRTIFIIS MYADSQPRGM AVAISVKSEC ISTLSSENKI |
| | C76S, C127S, | ISFKEMNPPD NIKDTKSDII FFQRSVPGHD NKMQFESSSY |
| | K70C | EGYFLASEKE RDLFKLILKK EDELGDRSIM FTVQNED |
| 56 | E6K, K53A, | YFGKLKSKLS VIRNLNDQVL FIDQGNRPLF EDMTDSDSRD |
| | C38S, C76S, | NAPRTIFIIS MYADSQPRGM AVTISVKCEK ISTLSSENKI |
| | C127S | ISFKEMNPPD NIKDTKSDII FFQRSVPGHD NKMQFESSSY |
| | | EGYFLASEKE RDLFKLILKK EDELGDRSIM FTVQNED |
| 57 | E6K, C38S, | YFGKLKSKLS VIRNLNDQVL FIDQGNRPLF EDMTDSDSRD |
| | K53A | NAPRTIFIIS MYADSQPRGM AVTISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 58 | E6K, K53A, | YFGKLKSKLS VIRNLNDQVL FIDQGNRPLF EDMTDSDSRD |
| | C38S, C68S, | NAPRTIFIIS MYADSQPRGM AVTISVKSEC ISTLSSENKI |
| | C76S, C127S, | ISFKEMNPPD NIKDTKSDII FFQRSVPGHD NKMQFESSSY |
| | K70C | EGYFLASEKE RDLFKLILKK EDELGDRSIM FTVQNED |
| 59 | E6K, C38A, | YFGKLKSKLS VIRNLNDQVL FIDQGNRPLF EDMTDSDARD |
| | K53A | NAPRTIFIIS MYADSQPRGM AVTISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 60 | E6K, C38Q, | YFGKLKSKLS VIRNLNDQVL FIDQGNRPLF EDMTDSDQRD |
| | K53A | NAPRTIFIIS MYADSQPRGM AVTISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 61 | E6K, C38A, | YFGKLKSKLS VIRNLNDQVL FIDQGNRPLF EDMTDSDARD |
| | K53A, C76A | NAPRTIFIIS MYADSQPRGM AVTISVKCEK ISTLSAENKI |
| | | ISFKEMNPPD NIKDTKSDII FFQRSVPGHD NKMQFESSSY |
| | | EGYFLACEKE RDLFKLILKK EDELGDRSIM FTVQNED |
| 62 | E6K, C38A, | YFGKLKSKLS VIRNLNDQVL FIDQGNRPLF EDMTDSDARD |
| | K53A, C127A | NAPRTIFIIS MYADSQPRGM AVTISVKCEK ISTLSCENKI |
| | | ISFKEMNPPD NIKDTKSDII FFQRSVPGHD NKMQFESSSY |
| | | EGYFLAAEKE RDLFKLILKK EDELGDRSIM FTVQNED |
| 63 | E6K, C38A, | YFGKLKSKLS VIRNLNDQVL FIDQGNRPLF EDMTDSDARD |
| | K53A, C76A, | NAPRTIFIIS MYADSQPRGM AVTISVKCEK ISTLSAENKI |
| | C127A | ISFKEMNPPD NIKDTKSDII FFQRSVPGHD NKMQFESSSY |
| | | EGYFLAAEKE RDLFKLILKK EDELGDRSIM FTVQNED |
| 64 | E6K, K53A, | YFGKLKSKLS VIRNLNDQVL FIDQGNRPLF EDMTDSDARD |
| | C38A, S55A, | NAPRTIFIIS MYADAQPRGM AVAISVKCEK ISTLSCENKI |
| | T63A | ISFKEMNPPD NIKDTKSDII FFQRSVPGHD NKMQFESSSY |
| | | EGYFLACEKE RDLFKLILKK EDELGDRSIM FTVQNED |
| 65 | E6K, C38Q, | YFGKLKSKLS VIRNLNDQVL FIDQGNRPLF EDMTDSDQRD |
| | K53A, S55A, | NAPRTIFIIS MYADAQPRGM AVAISVKCEK ISTLSCENKI |
| | T63A | ISFKEMNPPD NIKDTKSDII FFQRSVPGHD NKMQFESSSY |
| | | EGYFLACEKE RDLFKLILKK EDELGDRSIM FTVQNED |
| 66 | E6K, K53A, | YFGKLKSKLS VIRNLNDQVL FIDQGNRPLF EDMTDSDCRD |
| | K84A | NAPRTIFIIS MYADSQPRGM AVTISVKCEK ISTLSCENKI |
| | | ISFAEMNPPD NIKDTKSDII FFQRSVPGHD NKMQFESSSY |
| | | EGYFLACEKE RDLFKLILKK EDELGDRSIM FTVQNED |
| 67 | E6K, K53A, | YFGKLKSKLS VIRNLNDQVL FIDQGNRPLF EDMTDSDCRD |
| | D98A | NAPRTIFIIS MYADSQPRGM AVTISVKCEK ISTLSCENKI |
| | | ISFKEMNPPD NIKDTKSAII FFQRSVPGHD NKMQFESSSY |
| | | EGYFLACEKE RDLFKLILKK EDELGDRSIM FTVQNED |
| 68 | V11I, C38A, | YFGKLESKLS IIRNLNDQVL FIDQGNRPLF EDMTDSDARD |
| (a.k.a. | M51G, K53A, | NAPRTIFIIS GYADSQPRGM AVTISVKCEK ISTLSAENKI |
| C146) | C76A, C127A | ISFKEMNPPD NIKDTKSDII FFQRSVPGHD NKMQFESSSY |
| | | EGYFLAAEKE RDLFKLILKK EDELGDRSIM FTVQNED |
| 69 | E6K, V11I, | YFGKLKSKLS IIRNLNDQVL FIDQGNRPLF EDMTDSDARD |
| (a.k.a. | C38A, M51G, | NAPRTIFIIS GYADSQPRGM AVAISVKCEK ISTLSAENKI |
| C183) | K53A, T63A, | ISFKEMNPPD NIKDTKSDII FFQRSVPGHD NKMQFESSSY |
| | C76A, C127A | EGYFLAAEKE RDLFKLILKK EDELGDRSIM FTVQNED |
| 70 | N-terminal G, | GYFGKLKSKL SIIRNLNDQV LFIDQGNRPL FEDMTDSDAR |
| (a.k.a. | E6K, V11I, | DNAPRTIFII SGYADSQPRG MAVAISVKCE KISTLSAENK |
| C192) | C38A, M51G, | IISFKEMNPP DNIKDTKSDI IFFQRSVPGH DNKMQFESSS |
| | K53A, T63A, | YEGYFLAAEK ERDLFKLILK KEDELGDRSI MFTVQNED |
| | C76A, C127A | |
| 71 | N-terminal G, | GYFGKLKSKL SIIRNLNDQV LFIDQGNRPL FEDMTDSDAR |
| (a.k.a. | E6K, V11I, | DNAPRTIFII SMYADSQPRG MAVAISVKCE KISTLSAENK |
| C141) | C38A, K53A, | IISFKEMNPP DNIKDTKSDI IFFQRSVPGH DNKMQFESSS |
| | T63A, C76A, | YEGYFLAAEK ERDLFKLILK KEDELGDRSI MFTVQNED |
| | C127A | |
| 72 | N-terminal | GGGGYFGKLK SKLSIIRNLN DQVLFIDQGN RPLFEDMTDS |
| (a.k.a. | 4xG, E6K, | DARDNAPRTI FIISMYADSQ PRGMAVAISV KCEKISTLSA |
| C140) | V11I, C38A, | ENKIISFKEM NPPDNIKDTK SDIIFFQRSV PGHDNKMQFE |
| | K53A, T63A, | SSSYEGYFLA AEKERDLFKL ILKKEDELGD RSIMFTVQNE D |
| | C76A, C127A | |

### Additional Exemplary Il-18 Constructs

Also provided herein are IL-18 polypeptides which comprise the modifications to SEQ ID NO: 1 listed in the table below, each of which is assigned a Composition ID, which can be incorporated into a bifunctional cytokine composition as provided herein. In some embodiments, the IL-18 polypeptide of a bifunctional cytokine composition (e.g., an IL-2/IL-18 bifunctional cytokine composition) comprises the set of amino acid substitutions shown for any one of the constructs depicted below. In the constructs depicted below, each of the substitutions is listed using SEQ ID NO: 1 as a reference sequence. In some embodiments, the IL-18 polypeptide of bifunctional cytokine composition comprises only the substitutions shown for a construct below relative to SEQ ID NO: 1 (i.e., the IL-18 polypeptide has only the indicated set of substitutions and the remaining residues are those set forth in SEQ ID NO: 1).

TABLE 4′
Additional IL-18 Polypeptide

| Composition ID/ | | Composition ID/ | | Composition ID/ | |
|---|---|---|---|---|---|
| Substitutions to SEQ ID NO: 1 | | Substitutions to SEQ ID NO: 1 | | Substitutions to SEQ ID NO: 1 | |
| C143 | V11I, C38A, | C156 | V11I, C38A, N41A, | C168 | V11I, C38A, C76A, |
| | K53A, C76A, | | K53A, C76A, | | S105K, C127A |
| | C127A | | C127A | | |
| C144 | V11I, C38A, | C157 | V11I, C38A, K53A, | C174 | K8L, E6K, V11I, |
| | K53A, T63A, | | C76A, C127A, | | C38A, K53A, T63A, |
| | C76A, C127A | | D132A | | C76A, C127A |
| C145 | V11I, C38A, | C158 | V11I, C38A, K53A, | C175 | E6K, V11I, C38A, |
| | K53A, S55A, | | C76A, G108A, | | I49E, K53A, T63A, |
| | C76A, C127A | | C127A | | C76A, C127A |
| C147 | V11I, C38A, | C159 | V11I, C38A, K53A, | C176 | E6K, V11I, C38A, |
| | K53A, D54A, | | C76A, H109A, | | I49M, K53A, T63A, |
| | C76A, C127A | | C127A | | C76A, C127A |
| C148 | F2A, V11I, C38A, | C160 | V11I, C38A, K53A, | C177 | E6K, V11I, C38A, |
| | K53A, C76A, | | C76A, D110A, | | I49R, K53A, T63A, |
| | C127A | | C127A | | C76A, C127A |
| C149 | V11I, E31A, | C161 | K8R, V11I, C38A, | C178 | E6K, V11I, C38A, |
| | C38A, K53A, | | C76A, Q103E, | | K53A, T63A, C76A, |
| | C76A, C127A | | C127A | | Q103R, C127A |
| C150 | V11I, T34A, | C162 | K8E, V11I, C38A, | C179 | E6K, K8E, V11I, |
| | C38A, K53A, | | C76A, Q103R, | | C38A, K53A, T63A, |
| | C76A, C127A | | C127A | | C76A, Q103R, C127A |
| C151 | V11I, D35A, | C163 | V11I, C38A, C76A, | C180 | E6K, V11I, C38A, |
| | C38A, K53A, | | Q103K, C127A | | K53A, T63A, C76A, |
| | C76A, C127A | | | | C127A, V153R |
| C152 | V11I, S36A, C38A, | C164 | V11I, C38A, S55H, | C181 | E6K, V11I, C38A, |
| | K53A, C76A, | | C76A, C127A | | K53A, T63A, C76A, |
| | C127A | | | | C127A, V153E |
| C153 | V11I, D37A, | C165 | V11I, C38A, S55R, | C182 | E6K, V11I, C38A, |
| | C38A, K53A, | | C76A, C127A | | K53A, T63A, C76A, |
| | C76A, C127A | | | | C127A, V153Y |
| C154 | V11I, E31A, | C166 | V11I, C38A, S55T, | C184 | E6R, V11I, C38A, |
| | D37A, C38A, | | C76A, C127A | | K53A, T63A, C76A, |
| | K53A, C76A, | | | | C127A |
| | C127A | | | | |
| C155 | V11I, C38A, | C167 | V11I, C38A, C76A, | C142 | Y1M, E6K, V11I, |
| | D40A, K53A, | | S105I, C127A | | C38A, K53A, T63A, |
| | C76A, C127A | | | | C76A, C127A |

TABLE 4A
(IL-2 Polypeptides)

| SEQ | |
|---|---|
| ID | |
| NO | Sequence |
| 300 | APTSSSTKKTQLQLEHLLLDLQ-Nle-ILNGINNYKNPKLTR-Nle-L-Hse-Yn3-K |
| | F-Ygp-NlePKKATELKHLQCLEEELKPLEEVL-Hse-LAQSKNFHLRPRDLISNI |
| | NVIVLELKGSETTF-Hse-CEYADETATIVEFLNRWITFSQSIISTLT |
| 301 | APTSSSTKKTQLQLEHLLLDLQMILNGINNYKNPKLTRMLTFKFYMPKK |
| | ATELKHLQCLEEELKPLEEVLNLAQSKNFHLRPRDLISNINVIVLELKGSE |
| | TTFMCEYADETATIVEFLNRWITFCQSIISTLT |
| 302 | PTSSSTKKTQLQLEHLLLDLQMILNGINNYKNPKLTRMLTFKFYMPKKA |
| | TELKHLQCLEEELKPLEEVLNLAQSKNFHLRPRDLISNINVIVLELKGSET |
| | TFMCEYADETATIVEFLNRWITFSQSIISTLT |
| 303 | APTSSSTKKTQLQLEHLLLDLQ(Nle)ILNGINNYKNPKLTR(Nle)L(Hse)YK |
| | FY(Nle)PKKATELKHLQCLEEELKPLEEVL(Hse)LAQSKNFHLRPRDLISNI |
| | NVIVLELKGSETTF(Hse)CEYADETATIVEFLNRWITFSQSIISTLT |
| 304 | APTSSSTKKTQLQLEHLLLDLQ(Nle)ILNGINNYKNPKLTR(Nle)L(Hse)FK |
| | FY(Nle)PKKATELKHLQCLEEELKPLEEVL(Hse)LAQSKNFHLRPRDLISNI |
| | NVIVLELKGSETTF(Hse)CEYADETATIVEFLNRWITFSQSIISTLT |
| 305 | APTSSSTKKTQLQLEHLLLDLQ(Nle)ILNGINNYKNPKLTR(Nle)L(Hse)YK |
| | FY(Nle)PKKATELKHLQCLEEELKPLEEVL(Hse)YAQSKNFHLRPRDLISNI |
| | NVIVLELKGSETTF(Hse)CEYADETATIVEFLNRWITFSQSIISTLT |
| 306 | APTSSSTKKTQLQLEHLLLDLQ(Nle)ILNGINNYKNPKLTR(Nle)L(Hse)FK |
| | FY(Nle)PKKATELKHLQCLEEELKPLEEVL(Hse)YAQSKNFHLRPRDLISNI |
| | NVIVLELKGSETTF(Hse)CEYADETATIVEFLNRWITFSQSIISTLT |
| 307 | APTSSSTKKTQLQLEHLLLDLQ(Nle)ILNGINNYKNPKLTR(Nle)L(Hse)YK |
| | FY(Nle)PKKATELKHLQCLEEELKPLEEVL(Hse)GAQSKNFHLRPRDLISNI |
| | NVIVLELKGSETTF(Hse)CEYADETATIVEFLNRWITFSQSIISTLT |
| 308 | APTSSSTKKTQLQLEHLLLDLQ(Nle)ILNGINNYKNPKLTR(Nle)L(Hse)YK |
| | FY(Nle)PKKATELKHLQCLEEELKYLEEVL(Hse)LAQSKNFHLRPRDLISNI |
| | NVIVLELKGSETTF(Hse)CEYADETATIVEFLNRWITFSQSIISTLT |
| 309 | APTSSSTKKTQLQLEHLLLDLQ(Nle)ILNGINNYKNPKLTR(Nle)L(Hse)FK |
| | FY(Nle)PKKATELKHLQCLEEELKYLEEVL(Hse)LAQSKNFHLRPRDLISNI |
| | NVIVLELKGSETTF(Hse)CEYADETATIVEFLNRWITFSQSIISTLT |
| 310 | APTSSSTKKTQLQLEHLLLDLQ(Nle)ILNGINNYKNPKLTY(Nle)L(Hse)YK |
| | FY(Nle)PKKATELKHLQCLEEYLKYLEEVL(Hse)LAQSKNFHLRPRDLISN |
| | INVIVLELKGSETTF(Hse)CEYADETATIVEFLNRWITFSQSIISTLT |
| 311 | APTSSSTKKTQLQLEHLLLDLQ(Nle)ILNGINNYKNPKLTR(Nle)L(Hse)FK |
| | FY(Nle)PKKATELKHLQCLEEYLKYLEEVL(Hse)LAQSKNFHLRPRDLISN |
| | INVIVLELKGSETTF(Hse)CEYADETATIVEFLNRWITFSQSIISTLT |
| 312 | APTSSSTKKTQLQLEHLLLDLQ(Nle)ILNGINNYKNPKLTR(Nle)L(Hse)FK |
| | FY(Nle)PKKATELKHLQCLEEYLKPLEEVL(Hse)LAQSKNFHLRPRDLISNI |
| | NVIVLELKGSETTF(Hse)CEYADETATIVEFLNRWITFSQSIISTLT |
| 313 | APTSSSTKKTQLQLEHLLLDLQ(Nle)ILNGINNYKNPKLTR(Nle)L(Hse)FY |
| | FY(Nle)PKKATELKHLQCLEEELKPLEEVL(Hse)LAQSKNFHLRPRDLISNI |
| | NVIVLELKGSETTF(Hse)CEYADETATIVEFLNRWITFSQSIISTLT |
| 314 | APTSSSTKKTQLQLEHLLLDLQ(Nle)ILNGINNYKNPKLTR(Nle)L(Hse)FK |
| | YY(Nle)PKKATELKHLQCLEEELKPLEEVL(Hse)LAQSKNFHLRPRDLISNI |
| | NVIVLELKGSETTF(Hse)CEYADETATIVEFLNRWITFSQSIISTLT |
| 315 | APTSSSTKKTQLQLEHLLLDLQ(Nle)ILNGINNYKNPYLTR(Nle)L(Hse)FK |
| | FY(Nle)PKKATELKHLQCLEEELKPLEEVL(Hse)LAQSKNFHLRPRDLISNI |
| | NVIVLELKGSETTF(Hse)CEYADETATIVEFLNRWITFSQSIISTLT |
| 316 | APTSSSTKKTQLQLEYLLLDLQ(Nle)ILNGINNYKNPKLTR(Nle)L(Hse)FK |
| | FY(Nle)PKKATELKHLQCLEEELKPLEEVL(Hse)LAQSKNFHLRPRDLISNI |
| | NVIVLELKGSETTF(Hse)CEYADETATIVEFLNRWITFSQSIISTLT |
| 317 | APTSSSTKKTQLQLEYLLLDLQ(Nle)ILNGINNYKNPKLTR(Nle)L(Hse)YK |
| | FY(Nle)PKKATELKHLQCLEEELKPLEEVL(Hse)LAQSKNFHLRPRDLISNI |
| | NVIVLELKGSETTF(Hse)CEYADETATIVEFLNRWITFSQSIISTLT |
| 318 | APTSSSTKKTQLQLEHLLLYLQ(Nle)ILNGINNYKNPKLTR(Nle)L(Hse)FK |
| | FY(Nle)PKKATELKHLQCLEEELKPLEEVL(Hse)LAQSKNFHLRPRDLISNI |
| | NVIVLELKGSETTF(Hse)CEYADETATIVEFLNRWITFSQSIISTLT |
| 319 | APTSSSTKKTQLQLEHLLLYLQ(Nle)ILNGINNYKNPKLTR(Nle)L(Hse)YK |
| | FY(Nle)PKKATELKHLQCLEEELKPLEEVL(Hse)LAQSKNFHLRPRDLISNI |
| | NVIVLELKGSETTF(Hse)CEYADETATIVEFLNRWITFSQSIISTLT |
| 320 | APTSSSTKKTQLQLEHLLLDLQ(Nle)ILNGINNYKNPYLTR(Nle)L(Hse)YK |
| | FY(Nle)PKKATELKHLQCLEEELKPLEEVL(Hse)LAQSKNFHLRPRDLISNI |
| | NVIVLELKGSETTF(Hse)CEYADETATIVEFLNRWITFSQSIISTLT |
| 321 | APTSSSTKKTQLQLEHLLLDLQ(Nle)ILNGINNYKNPKLTR(Nle)L(Hse)YK |
| | FY(Nle)PKKATELKHLQCLEEYLKPLEEVL(Hse)LAQSKNFHLRPRDLISNI |
| | NVIVLELKGSETTF(Hse)CEYADETATIVEFLNRWITFSQSIISTLT |
| 322 | APTSSSTKKTQLQLEHLLLDLQ(Nle)ILNGINNYKNPKLTR(Nle)L(Hse)YK |
| | FY(Nle)PKKATELKHLQCLEEYLKYLEEVL(Hse)LAQSKNFHLRPRDLISN |
| | INVIVLELKGSETTF(Hse)CEYADETATIVEFLNRWITFSQSIISTLT |
| 323 | APTSSSTKKTQLQLEHLLLDLQ(Nle)ILNGINNYKNPKLTR(Nle)L(Hse)FK |
| | FY(Nle)PKKATELKHLQCLEEELKPLEEVL(Hse)LAQSKNFHLRPRDLISNI |
| | NVIVLELKGSETTF(Hse)CEYADETATIVEFLNRWITFCQSIISTLT |

In Table 4A above, Nle is a norleucine residue, Hse is a homoserine residue, Yn3 is a tyrosine residue modified with an azide-capped PEG9 group (see below), and Ygp is a tyrosine residue modified with an amino-capped PEG8 group (see below):

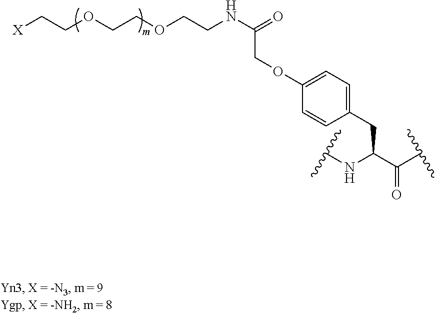

TABLE 4B
(IL-7 Polypeptides)

| SEQ | |
|---|---|
| ID NO | IL-7 Sequence |
| 324 | MFHVSFRYIFGLPPLILVLLPVASSDCDIEGKDG |
| | KQYESVLMVSIDQLLDSMKEIGSNCLNNEFNFFK |
| | RHICDANKEGMFLFRAARKLRQFLKMNSTGDFDL |
| | HLLKVSEGTTILLNCTGQVKGRKPAALGEAQPTK |
| | SLEENKSLKEQKKLNDLCFLKRLLQEIKTCWNKI |
| | LMGTKEH |
| 325 | MFHVSFRYIFGIPPLILVLLPVTSSDCHIKDKDG |
| | KAFGSVLMISINQLDKMTGTDSDCPNNEPNFFKK |
| | HLCDDTKEAAFLNRAARKLRQFLKMNISEEFNDH |
| | LLRVSDGTQTLVNCTSKEEKTIKEQKKNDPCFLK |
| | RLLREIKTCWNKILKGSI |
| 326 | MFHVSFRYIFGIPPLILVLLPVTSSECHIKDKEG |
| | KAYESVLMISIDELDKMTGTDSNCPNNEPNFFRK |
| | HVCDDTKEAAFLNRAARKLKQFLKMNISEEFNVH |
| | LLTVSQGTQTLVNCTSKEEKNVKEQKKNDACFLK |
| | RLLREIKTCWNKILKGSI |
| 327 | MFHVSFRYIFGIPPLILVLLPVASSDCDISGKDG |
| | GAYQNVLMVNIDDLDNMINFDSNCLNNEPNFFKK |
| | HSCDDNKEASFLNRASRKLRQFLKMNISDDFKLH |
| | LSTVSQGTLTLLNCTSKGKGRKPPSLSEAQPTKN |
| | LEENKSSKEQKKQNDLCFLKILLQKIKTCWNKIL |
| | RGIKEH |
| 328 | MFHVSFRYIFGIPPLILVLLPVASSDCDFSGKDG |
| | GAYQNVLMVSIDDLDNMINFDSNCLNNEPNFFKK |
| | HSCDDNKEASFLNRAARKLKQFLKMNISDDFKLH |
| | LSTVSQGTLTLLNCTSKGKGRKPPSLGEAQPTKN |
| | LEENKSLKEQRKQNDLCFLKILLQKIKTCWNKIL |
| | RGITEH |

The present disclosure is further illustrated in the following Examples which are given for illustration purposes only and are not intended to limit the disclosure in any way.

### EXAMPLES

### Example 1A: Preparation of IL-18 Polypeptides

IL-18 variants suitable for linking to another cytokine as provided herein can be prepared according to the protocols provided below. In some instances, the IL-18 will contain a cysteine residue at the desired point of attachment of the linker, or may include an unnatural amino acid (e.g., azidolysine) or modified natural amino acid suitable for attachment of the linker at the desired point of attachment. The expression systems used below for preparation of recombinant IL-18 can be replaced with an expression system with an orthogonal or expanded genetic code for use in incorporating amino acids comprising conjugation handles.

### Recombinant IL-18 Preparation

Soluble his-SUMO-IL18 Variants

E. coli BL21 (DE3) harboring a plasmid encoding a N-His-SUMO tagged IL-18 variant fusion is inoculated into 3 L LB culture medium and induced with 0.4 mM IPTG at 30° C. for 6 h. Cells are pelleted and cell lysis is done by sonication in lysis buffer: PBS, pH 7.4. Soluble protein is purified via Ni-NTA beads 6FF (wash 1 with: PBS, 20 mM imidazole, pH7.4; wash 2 with PBS, 50 mM Imidazole, pH7.4; elution with PBS, 500 mM imidazole, pH7.4).

Fractions containing the protein are pooled, dialyzed into PBS pH 7.4 and followed by SUMO digestion. Then the protein is two-step purified with Ni-NTA beads (continue with flow through sample) and gel filtration. Fractions containing the protein are pooled and QC is performed using analytical techniques, such as SDS-PAGE and analytical SEC.

Insoluble his-SUMO-IL18 Variants

E. coli BL21 (DE3) harboring a plasmid encoding a N-His-SUMO tagged IL-18 variant fusion are inoculated into 10 L LB culture medium and induced with 0.4 mM IPTG at 30° C. for 6 h. Cells are pelleted and cell lysis is done by sonication in lysis buffer: PBS, 8 M urea, pH 7.4. Protein is purified via Ni-NTA beads 6FF (wash 1 with: PBS, 8 M urea, 20 mM imidazole, pH7.4; wash 2 with PBS, 8 M urea, 50 mM Imidazole, pH7.4; elution with PBS, 8 M urea, 500 mM imidazole, pH7.4).

Fractions containing the protein are pooled, dialyzed into PBS pH 7.4 and followed by SUMO digestion. Then the protein is purified with Ni-NTA beads (equilibrate column with PBS, 8 M urea, pH 7.4, wash with PBS, 8 M urea, pH 7.4, elution with PBS, 8 M urea, pH 7.4). Fractions containing the protein are pooled, dialyzed into PBS pH 7.4 and QC is performed using analytical techniques, such as SDS-PAGE and analytical SEC.

### Insoluble Tagless IL18 Variants

E. coli BL21 (DE3) harboring a plasmid encoding mIL-18 is inoculated into 2 L LB culture medium and induced with 0.4 mM IPTG at 30° C. for 6 h. Cells are pelleted and cell lysis was done by sonication in lysis buffer: 110 mM Tris, 1.1 M guanidine HCl, 5 mM DTT, pH 8.9. Protein as purified via Q Sepharose FF (balance buffer 20 mM MES, pH 7.0, elution with an increasing gradient from 0 to 1 M NaCl).

### Bicistronic System

A single colony of E. coli BL21 containing the plasmid (e.g., SEQ ID: 59) is used as an inoculum for 10 mL LB containing 25 μg/mL kanamycin sulfate and incubated overnight at 37° C. and 200 rpm. 1 mL of the preculture are used to inoculate 1 L autoinducing terrific broth containing 100 μg/mL kanamycin sulfate. The culture is incubated at 37° C. and 110 rpm for 4 h and then transferred to 15° C. for another 15 h. Cells are resuspended in 10-15 mL lysis buffer (100 mM HEPES, 1 mM EDTA, 5 mM DTT, 20 μg/mL lysozyme, 0.1 mg/mL DNase I, 1 mM PMSF, pH 7.5) and gently shaken at 4° C. for 1 h. Then the cells are lysed with sonication and the soluble protein fraction is obtained by centrifugation (16′000×g, 30 min, 4° C.) and filtration (0.2 μm membrane).

The supernatant is adjusted to ca. pH 7 and loaded on a tandem column system (2×SP CIEX+1× HiPrep DEAE FF 16/10, all from cytiva) using a 50 mL superloop (loading less than 30 mL lysate per run). The system is run with wash buffer (25 mM HEPES, 1 mM EDTA, 5 mM DTT, pH 7.0) and fractions containing the protein (second main peak) are collected and pooled.

The tandem columns are separated into their respective types. The DEAE columns were eluted with buffers E1 and E2 (25 mM Bus-Tris Propane HCl, pH 9.5 and 25 mM Bis-Tris Propane HCl, 1 M NaCl, pH 9.5 respectively) with a stepwise gradient. First, 100% E1 was run for 8 CV, followed by a gradient from 0% to 12% E2 over 5 CV and then keeping it at 12% for another 10 CV. This is followed by a gradient from 12% to 40% E2 over 5 CV and keeping it at 40% for another 5 CV. Fractions containing the protein (second main peak) are collected and pooled with the previous fractions. The SP columns are washed with the same method and discard, as no protein should be found in this elution.

The pooled samples are adjusted to pH 9.5 and loaded on a Mono Q (small scale) or Hitrap Q (large scale) column. Buffers used are E2 and E3 (25 mM Bis-Tris Propane HCl, 1.5 M Ammonium Sulfate, pH 9.5). The stepwise elution gradient starts at 8% E3 for 15 CV, increasing to 16% E3 over 5 CV and the increasing to 50% E3 over 3 CV. Fractions containing the protein are found in the second main peak.

The fractions containing the target protein are pooled and concentrated by diafiltration (10 kDa MWCO, less than 3500×g, 4° C.). The concentrated sample is loaded on a Superdex 75 equilibrated with buffer (20 mM potassium phosphate, 150 mM KCl, 1 mM DTT, pH 6.0). Fractions containing the target protein are collected, pooled and concentrated.

### Example 1B—Additional Methods for Recombinant IL18 Expression and Purification

The following protocols were also used to prepare certain IL-18 polypeptides provided herein which were subsequently used either in assays for conversion into bifunctional cytokine compositions as provided herein.

### Expression of IL-18 Polypeptides

IL-18 polypeptide were produced as an N-terminal fusion to N-His-SUMO-IL18. The gene was synthesized and cloned by a commercial vendor. Plasmids were transformed into E. coli BL21 (DE3). Expression was performed in shake flasks with TB medium. The cells were grown at 37° C. until an OD600 of approximately 1.2 was reached, after which they were induced by 0.1 mM IPTG and cultured for another 20 hours at 18° C. Cells were harvested by centrifugation.

Purification of IL18 Polypeptides

Cell lysis—Cells were resuspended in lysis buffer (20 mM Tris/HCl, pH 8.0, 0.15 M NaCl, 10 mM Imidazole, 1 tablet of EDTA-free complete protease inhibitor (Roche, COEDTAF-RO) per liter production) at 100 mL buffer/L culture and disrupted twice with a homogenizer at 1000 bar. The lysate was cleared of debris by centrifugation at 40′000 g for 2×45 minutes, changing flask in between, and subsequent filtration through a 0.22 μm filter.

Affinity Purification and Endotoxin Removal—The lysate was loaded on Ni NTA resin (Cytiva, 17524802) pre-equilibrated with 20 mM Tris/HCl, pH 8.0, 0.15 M NaCl, 10 mM Imidazole, at 5 mL/min and washed with the same buffer for 5 CV. To remove endotoxins, the column was washed with 20 mM Tris/HCl, pH 8.0, 0.15 M NaCl, 10 mM Imidazole, 0.1% Tryton X-114 at 10 mL/min for 30 CV. The column was washed with 20 mM Tris/HCl, pH 8.0, 0.15 M NaCl, 10 mM Imidazole, for 5 CV at 5 mL/min and the protein of interest eluted by linear increase of imidazole concentration. The column was then regenerated by 0.5M NaOH.

SUMO digestion and dialysis—To cleave the SUMO tag, SUMO protease was added to the elution pool at a w/w ratio of 1:250 (protein:SUMO enzyme) and incubated for 18 hours at 4° C. At the same time, the protein was dialysed (20 mM Tris, pH 8.0, 150 mM NaCl), to reduce the imidazole concentration.

Purification by reverse IMAC—In order to remove the cleaved tag and the SUMO protease, the digested protein was flown through a Ni NTA resin column pre-equilibrated with 20 mM Tris/HCl, pH 8.0, 0.15 M NaCl, 10 mM Imidazole, at 5 mL/min. The flow-through was collected.

Buffer Exchange—The flow-through was concentrated to 2.6 mg/mL and buffer exchanged into either 20 mM HEPES, 150 mM NaCl, 0.5 mM TCEP, 10% glycerol, pH7.5 or PBS, 10% glycerol, pH7.4. Proteins were stored at −70° C. until further quality controls.

### Example 2: Preparation of IL-2 Polypeptides

An IL-2 polypeptide suitable for preparation of a bifunctional cytokine composition as provided herein can be prepared by either recombinant or synthetic means known in the art.

Examples of suitable IL-2 polypeptides expressed recombinantly are described at least in PCT Publication No. WO2019028419A1, which describes recombinant expression of IL-2 polypeptides with amino acids comprising conjugation handles incorporated using an expression system with an orthogonal or expanded genetic code.

Examples of chemically synthesized IL-2 polypeptides suitable for the preparation of bifunctional cytokine composition are described at least in PCT Publication No. WO2021140416A2, which describes the chemical synthesis of IL-2 polypeptides by α-ketoacid-hydroxylamine (KAHA) ligation which incorporate amino acids comprising conjugation handles.

### Example 3: Preparation of IL-2/IL-18 Bifunctional Cytokine Compositions

Conjugation of IL-18 Polypeptide with Bifunctional Linking Group

An IL-18 polypeptide as provided herein can conjugated to a bifunctional linking group prior to forming the full linker of the bifunctional cytokine composition. Bifunctional linking group structures used herein are shown below. Other bifunctional linking groups could also be used.

### Linking Groups Used

| | Linker | | |
|---|---|---|---|
| Linking Group | No | MW | Structure |
| Bromoacetamido- PEG4-DBCO | 001 | 644.55 | 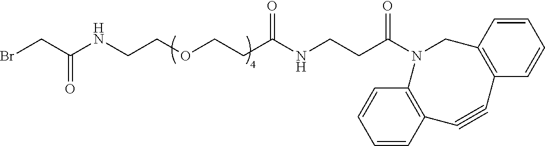 |
| Bromoacetamido- PEG12-DBCO | 002 | 996.97 | 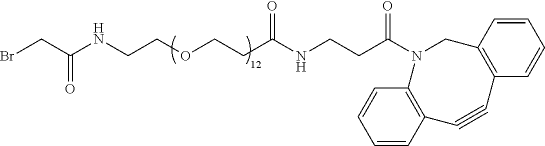 |
| Bromoacetamido- PEG24-DBCO | 003 | 1525.60 | 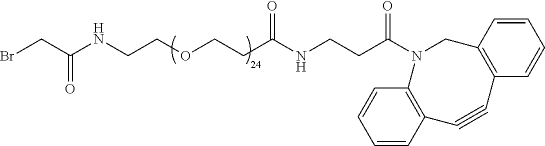 |
| SulfoDBCO- Maleimide | 004 | 578.60 | 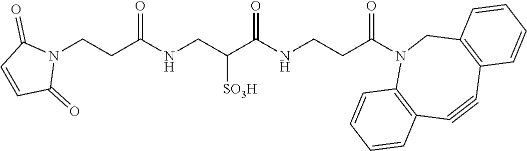 [SMILES: O=C(CCN1C(=O)C=CC1=O)NCC(C(=O)NCCC(=O)N1Cc2ccccc2C#Cc2ccccc21)S(=O)(=O)O] |
| SulfoDBCO-PEG4- Maleimide | 005 | 825.89 | 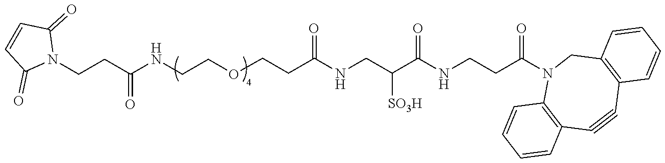 |
| SulfoDBCO-PEG24- Maleimide | 006 | 1555.81 | 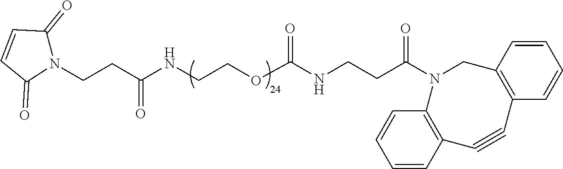 |

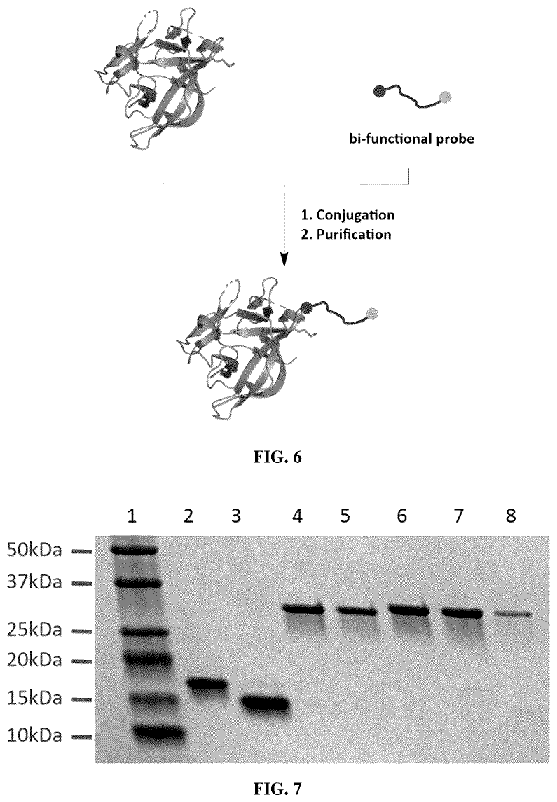

In some cases, the bifunctional linking group first attaches to a desired residue of the IL-18 polypeptide at the point of attachment of the linker. Once attached to the IL-18 polypeptide, the second functionality of the bifunctional linking group is used to attach to a second portion. An exemplary schematic of such a process is shown in FIG. 6. Not all modified IL-18 polypeptides suitable for inclusion in bifunctional cytokine compositions require binding of a bifunctional linking group as shown in FIG. 6. In some instances, modified IL-18 polypeptides already have the desired conjugation handle for forming a bifunctional cytokine composition (e.g., incorporated during synthesis or recombinantly expressed with an unnatural amino acid). An exemplary protocol on an IL-18 polypeptide with a cysteine residue point of attachment (e.g., residue C68 of SEQ ID NO: 1) provided herein is described below.

Conjugation—The IL-18 polypeptide is stored at a concentration of 2.4 mg/mL at −80° C. in potassium phosphate buffer (pH 7.0) containing 50 mM KCl and 1 mM DTT or in PBS pH7.4+10% glycerol. The sample is thawed on ice yielding a clear solution. The protein solution is diluted in PBS, pH 7.4. A clear solution is obtained at a concentration of ˜0.4 mg/mL.

The protein solution is dialyzed against PBS, pH 7.4 (twice against 600 mL for 2 h and once against 800 mL for 18 h). After dialysis, a clear solution is obtained with no sign of precipitation. Protein concentration is obtained using UV absorbance at 280 nm and by BCA protein assay. A stock solution of bi-functional linking group (e.g., bromoacetamido-PEG4-DBCO, Product #: 11221 from Quanta Biodesign Ltd) in water is prepared at a concentration of 20 mM. 500 μL of the protein solution are mixed with 25 μL of linking group solution. pH was adjusted to 7.5 and it was let to react for 3 h at 20° C.

The progress of the synthesis is monitored by reverse-phase HPLC using a gradient of 5 to 30% (2.5 min) and 30 to 75% (7.5 min) CH₃CN with 0.1% TFA (v/v) on a Aeris WIDEPORE C18 200 Å column (3.6 μm, 150×4.6 mm) at a flow rate of 1 mL/min at 40° C. and by MALDI-TOF MS.

Purification—In some cases, ion-exchange chromatography is used to purify the conjugated protein. To remove the excess of probe, the reaction mixture (volume is around 500 μL) is flowed through a Hi-Trap-Q-FF-1 mL column using 25 mM Tris (pH 7.4) as the buffer. The column is eluted with a linear gradient of 0-0.35 M NaCl in the same buffer. The fractions containing the target protein are gathered, buffer exchanged (25 mM Tris, pH 7.4, 75 mM NaCl) and concentrated at 0.4 mg/mL. The concentration of purified protein is determined by UV absorbance at 280 nm and by BCA protein assay. The protein solution is kept at −80° C.

Characterization—The purity and identity of the recombinant protein from commercial source and the conjugated protein is confirmed by analytical SEC, HPLC and MALDI-TOF MS.

### Conjugation of IL-18 Polypeptide to IL-2 Polypeptide

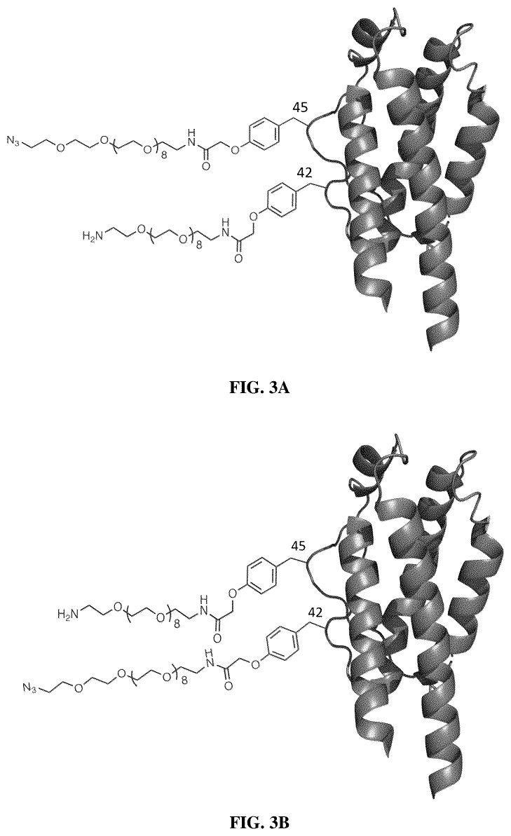

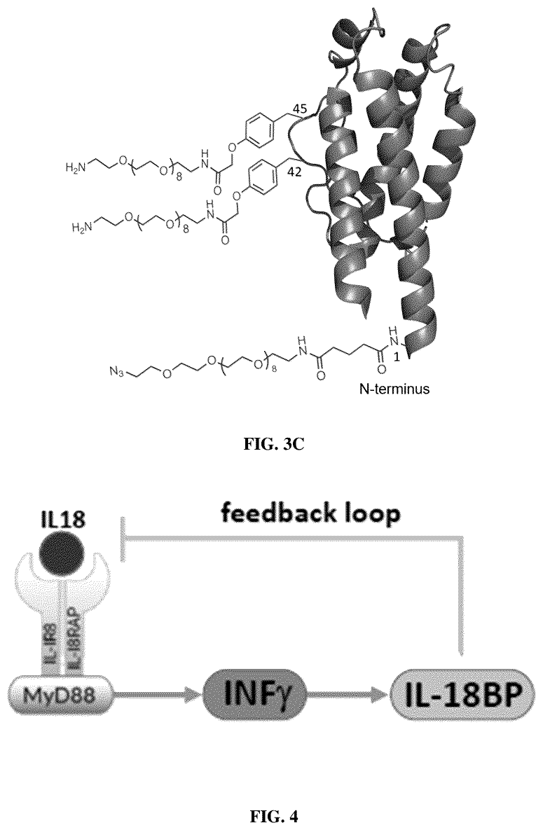

An IL-18 polypeptide comprising a DBCO conjugation handle (e.g., an IL-18 polypeptide conjugated to a bifunctional reagent as described supra or an IL-18 polypeptide comprising an amino acid residue comprising a DBCO conjugation, such as an unnatural or modified amino acid provided herein) is conjugated to an IL-2 polypeptide comprising an azide conjugation handle provided herein. Exemplary IL-2 polypeptides include those depicted in FIG. 3A (IL-2 polypeptide with conjugation handle attached at residue Y45), FIG. 3B (IL-2 polypeptide with conjugation handle attached at residue F42Y), and/or FIG. 3C (IL-2 polypeptide with conjugation handle attached at N-terminal amine).

Briefly, DBCO containing IL-18 is reacted with 2-10 equivalents of azide containing IL-2 (e.g., pH 5.2 buffer (e.g., sodium acetate), 5% trehalose, rt, 24 h). Progress of the reaction is monitored by HPLC. The resulting IL-2/IL-18 bifunctional cytokine composition is purified by cation-exchange chromatography, anion-exchange chromatography, and/or size exclusion chromatography to obtain purified bifunctional cytokine composition. IL-2/IL-18 bifunctional cytokine composition is purified from unreacted IL-18 and aggregates using a desalting column, CIEX and SEC (e.g., GE Healthcare Life Sciences AKTA pure, mobile phase: Histidine 5.2/150 mM NaCV/5% Trehalose, column: GE Healthcare Life Sciences SUPERDEX™ 200 increase 3.2/300, flow rate: 0.5 mL/min). Alternatively, IL-2/IL-18 bifunctional cytokine composition is instead purified from unreacted IL-18 and aggregates using AIEX with a Tris-based buffer (instead of or in addition to other purification steps).

The purity and identity of the IL-2/IL-18 bifunctional cytokine composition is confirmed by Rβ-HPLC (HPLC: ThermoFisher Scientific UHPLC Ultimate 3000, column: Waters BEH C-4 300A, 3.0 μm, 4.6 mm, 250 mm, mobile phase A: 0.05% TFA in Water, mobile phase B: 0.05% TFA in mixture of ACN:IPA:ETOH:H2O (5:1.5:2:1.5), flow rate: 0.5 mL/min, injection amount: 10 μg (10 μL Injection of 1 mg/mL), gradient: 0% to 20% mobile phase B in 50 min) and SDS-PAGE.

The resulting purified IL-2/IL-18 bifunctional cytokine composition is then tested for its IL-2 and IL-18 associated activities as provided below.

### Example 3B—Exemplary Bifunctional Cytokine Compositions Prepared Herein

The bifunctional cytokine compositions provided in the table below were prepared according to the following general protocols.

### List of Generated and Tested Bifunctional Cytokine Compositions.

| Composition ID NO: | IL-18 payload | Linker | IL-2 payload |
|---|---|---|---|
| 1 | SEQ ID NO: 30 | 003 | SEQ ID NO: 300 |
| 2 | SEQ ID NO: 30 | 005 | SEQ ID NO: 300 |
| 3 | SEQ ID NO: 30 | 006 | SEQ ID NO: 300 |
| 4 | SEQ ID NO: 30 | 004 | SEQ ID NO: 300 |
| 5 | SEQ ID NO: 3 | 005 | SEQ ID NO: 300 |

Bifunctional linking reagents (i.e., Linkers 001-006 provided herein) were prepared as 40 mM stock solutions in MilliQ water or PBS pH 7.4. Linker 001 and 002 did not dissolve well and could only be used as turbid suspensions. As a first step either IL-2 of SEQ ID NO: 300 or IL-18 polypeptide of SEQ ID NO: 30 or 300 were incubated with 2-80 eq of bifunctional linking reagent 20-24° C. Reactions were incubated between 2 and 4 hours. Linker 004 and 005 could be separated from the protein fraction by a desalting run on the AKTA™ protein purification system (Cytiva), while bigger linkers needed anion exchange chromatography using Capto Q ImpRes (Cytiva) resin (for IL-18 polypeptides of SEQ ID NO: 3 and 30) or cation exchange chromatography using Capto S ImpAct (Cytiva) resin (for IL-2 polypeptide of SEQ ID NO: 300) to separate excess linker from the protein.

The second step to form the bifunctional cytokine composition involved taking the modified protein (i.e., the IL-2 polypeptide of SEQ ID NO: 300 or the IL-18 polypeptide of SEQ ID NO: 3 or 30 reacted with bifunctional linking group) and mixing it with the other protein (i.e., the protein not reacted with the bifunctional linking group) (roughly 1 eq). Reactions were incubated overnight at 24° C. All resulting bifunctional cytokine compositions were purified using Capto™ Q ImpRes resin according to manufacturer's instructions.

The resulting bifunctional cytokine compositions were then analyzed and checked for purity by Rβ-HPLC (Phenomenex Aeris 3.6 μm Widepore C4 200 Å; 4.6×150 mm with water+0.1% TFA (v/v) and acetonitrile+0.1% TFA (v/v) as buffers A and B) and SE-HPLC (either TSKgel UP-SW3000 column 4.6×300 mm, 2 μm, 25 nm pore size, CV: 4.99 mL (TOSOH BIOSCIENCE) or XBridge BEH200 Å SEC column 7.8×300 mm, 3.5 μm, 200 Å pore size (Waters) as the column with PBS pH 7.2 as running buffer). For each bifunctional cytokine composition, the HPLC traces showed that each was prepared in high purity and eluted as a smooth single peak. The bifunctional cytokine compositions were also analyzed by SDS-PAGE (FIG. 7). In FIG. 7, lane 1 is MW marker, lane 2 is SEQ ID NO: 30, lane 3 is SEQ ID NO: 300, lane 4 is Composition 2 (batch 1), lane 5 is Composition 2 (batch 2), lane 6 is Composition 3 (batch 1), lane 7 is Composition 3 (batch 2), and lane 8 is Composition 1.

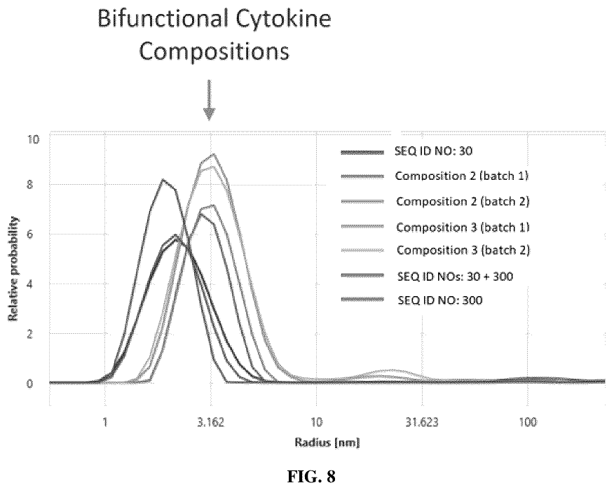

The bifunctional cytokine compositions were also analyzed by dynamic light scattering (DLS) in order to assess particle size and uniformity (FIG. 8). The arrow in the figure indicates the peaks associated with the bifunctional cytokine compositions tested (Compositions 1 and 2, both batches), all of which are centered around 3.162 nm, whereas the individual cytokines (or combination) shows smaller peak radii. Cumulant radius and cumulant polydispersity index results for bifunctional cytokine compositions and precursor cytokines is provided in the table below.

| | Cumulant radius | Cumulant polydispersity |
|---|---|---|
| Construct | (nm) | index |
| SEQ ID NO: 300 | 1.9 | 0.13 |
| SEQ ID NO: 30 | 2.2 | 0.33 |
| SEQ ID NO: 300 + | 2.7 | 0.58 |
| SEQ ID NO: 30 | | |
| Composition 2 (Batch 1) | 3.1 | 0.02 |
| Composition 2 (Batch 2) | 3.20 | 0.05 |
| Composition 3 (Batch 1) | 3.2 | 0.11 |
| Composition 3 (Batch 2) | 3.2 | 0.22 |

Compositions were also confirmed by mass spec (MALDI-MS or HRMS) as indicated in the table below. Variations in the theoretical and measured MW from MALDI-MS method are likely due to instrument error from the measured MW being at the limit of the instrument used.

| Bifunctional Cytokine | Theoretical MW | Measured MW | |
|---|---|---|---|
| Composition | (Da) | (Da) | MS Method |
| 1 | 35954.1 | 36185.3 | MALDI-MS |
| 2 | 35253.3 | 35253.8 | HRMS |
| 3 | 35983.8 | 35983.4 | HRMS |
| 4 | 35006.1 | 35005.5 | HRMS |
| 5 | 35195.9 | 35231.8 | MALDI-MS |

### Example 4: Characterization of Bifunctional Cytokine Compositions (Bioassays)

A bifunctional cytokine composition provided herein is tested for activities and binding associated with the individual cytokine components. For example, a bifunctional cytokine composition comprising an IL-18 polypeptide conjugated to an IL-2 polypeptide is tested according to the protocols described below to assess, for example, binding to one or more IL-18 receptor subunits, binding to one or more IL-2 receptor subunits, and other bioactivities associated with each cytokine individually.

### 4.1 IL18 Binding and Functional Assays

The ability of the bifunctional cytokine composition to perform various IL-18 activities is measured as provided below, as well as relevant comparisons to non-conjugated IL-18 polypeptides

### 4.1.1 Surface Plasmon Resonance

The interaction of bifunctional cytokine composition, wild type IL-18, and/or modified IL-18 polypeptides with human IL-18 receptor subunits are measured with Surface Plasmon Resonance (SPR) technology. Anti-human IgG antibodies are bound by amine coupling onto a CM5 chip to capture 6 μg/mL of Fc fused human IL-18Rα, 6 μg/mL of Fc fused human IL-18Rβ, or 2 μg/mL of Fc fused human IL-18BP isoform a (IL-18BPa) for 30 min before capture. In other settings, 6 μg/mL of alpha and beta IL-18 receptors are mixed and pre-incubated for 30 min before capture of the alpha/beta heterodimer IL-18 receptor.

The kinetic binding of the IL-18 the bifunctional cytokine composition and, analytes are measured with a Biacore 8K instrument in two-fold serial dilutions starting at 1 μM down to 0.98 nM. Regeneration of the surface back to amine coupled anti IgG antibody is done after every concentration of analyte. To measure the protein association to the receptors, the samples are injected with a flow rate of 50 μL/min for 60 s, followed by 300 s buffer only to detect the dissociation. The used running buffer is 1×PBS with 0.05% Tween20. The relative response units (RU, Y-axis) are plotted against time (s, X-axis) and analyzed in a kinetic 1:1 binding model for the monomer receptor binding and for the binding to the IL-18BP. A kinetic heterogenous ligand fit model is applied for the alpha/beta heterodimer binding. It is expected that bifunctional cytokine compositions retain binding to the IL-18 receptor or subunits thereof similarly or only slightly reduced compared to IL-18 polypeptides not incorporated into bifunctional cytokine compositions. It is also expected that IL-2 receptor binding is similarly maintained.

4.1.2 IL-18BP Binding alphaLISA Assay

A human IL-18BP AlphaLISA Assay Kit is used to determine the binding affinity of the bifunctional cytokine composition for IL-18BP, which detected the presence of free form IL-18BP.

Sixteen three-fold serial dilutions of IL-18 analytes are prepared in aMEM medium supplemented with 20% FCS, Glutamax, and 25 μM β-mercaptoethanol in the presence of 5 ng/mL of His-tagged human IL-18BP. Final IL-18 analytes concentration range from 2778 nM to 0.2 pM.

After 1 hr incubation at room temperature, free IL-18BP levels are measured using a Human IFNγ AlphaLISA Assay Kit. In a 384 well OPTIplate, 5 μL of 5× Anti-IL-18BP acceptor beads are added to 7.5 μL of an IL-18/IL-18BP mix. After 30 min incubation at room temperature with shaking, 5 μL of biotinylated Anti-IL-18BP antibodies are added to each well. The plate is incubated further for 1 hr at room temperature. Under subdued light, 12.5 μL of 2× streptavidin (SA) donor beads are pipetted into each well, and the wells are incubated with shaking for an additional 30 min at room temperature. The AlphaLisa signal is then measured on an Enspire plate reader with 680 and 615 nm as excitation and emission wavelengths, respectively. The dissociation constant (K_{D}) is calculated based on a variable slope, four parameter analysis using GraphPad PRISM software.

### 4.1.3 IL-18 IFNγ Induction Cellular Assay

The ability of the bifunctional cytokine composition provided herein are assessed for ability to induce IFNγ in a cellular assay according to the protocol below.

The NK cell line NK-92 derived from a patient with lymphoma (ATCC® CRL-2407™) is cultured in aMEM medium supplemented with 20% FCS, Glutamax, 25 μM B-mercaptoethanol, and 100 IU/mL of recombinant human IL-2.

On the day of experiment, cells are harvested and washed with aMEM medium without IL-2 and containing 1 ng/mL of recombinant human IL-12. After counting, cells are seeded at 100,000 cells/well in a 384 well titer plate and incubated at 37° C./5% CO₂. Sixteen 4-fold serial dilutions of the bifunctional cytokine composition are prepared in aMEM medium, and 1 ng/mL of IL-12 are added to the NK-92 cells. Final bifunctional cytokine composition analyte concentrations range from 56 nM to 5×10⁻⁵ pM.

After incubating the cells for 16-20 hr at 37° C./5% CO₂, 5 μL of supernatant is carefully transferred to a 384 microwell OptiPlate. IFNγ levels are measured using a human IFNγ AlphaLISA Assay Kit. Briefly, 10 μL of 2.5× AlphaLISA Anti-IFNγ acceptor beads and biotinylated antibody anti-IFNγ mix are added to the 5 μL of NK-92 supernatants. The mixtures are incubated for 1 hr at room temperature with shaking. Under subdued light, 2.5 μL of 2× streptavidin (SA) donor beads are pipetted into each well, and the wells are incubated for 30 min at room temperature with shaking. AlphaLISA signals are then measured on an EnSpire™ plate reader using 680 nm and 615 nm as excitation and emission wavelengths, respectively. Half maximal effective concentrations (EC₅₀) are calculated based on a variable slope and four parameter analysis using GraphPad PRISM software.

### 4.1.3A IL-18 IFNγ Induction Cellular Assay Results

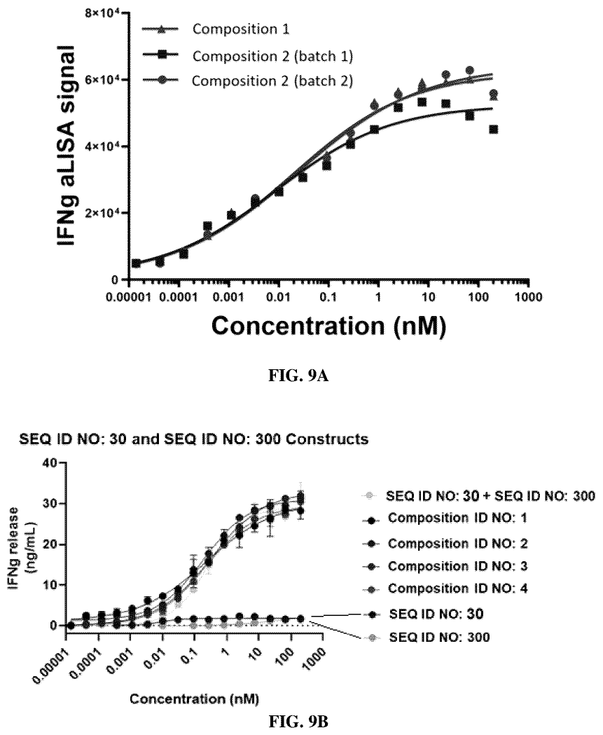

An experiment substantially identical to that described above was performed with bifunctional cytokine compositions provided herein and appropriate controls, with the difference that the NK92 cells were not stimulated with IL-12. Results are shown in the table below. An exemplary dose response curve of cells treated with Composition 1 (having a bromo-acetamide PEG₂₄ linker (˜90 angstroms)) and two batches of Composition 2 (having a maleimide PEG₄ linker (˜33 angstroms) in NK92 cells is shown in FIG. 9A. Both bifunctional cytokine compositions performed similarly, indicating minimal impact of linker chemistry or linker length on activity. Dose response curves of additional compositions in NK92 cells is shown in FIG. 9B, with all bifunctional cytokine compositions exhibiting similar results and the combination of IL-18 and IL-2 controls also showing efficacy, while IL-2 and IL-18 administered alone showed little IFNγ production (bottom lines on plot).

TABLE A
Results of IFNγ stimulation assay

| Bifunctional Cytokine | NK92 IFNγ |
|---|---|
| Composition/Cytokine | EC50 (nM) |
| Composition 1 | 0.26 |
| Composition 2 | 0.16 |
| Composition 3 | 0.22 |
| Composition 4 | 0.19 |
| Composition 5 | 0.25 |
| SEQ ID NO: 300 | 25.48 |
| SEQ ID NO: 30 | 0.0048 |
| SEQ ID NO: 300 + SEQ | 0.26 |
| ID NO: 30 | |

### 4.1.3B IL-18 IFNγ Induction Cellular Assay Results—PBMCs

An experiment analagous to that described in Example 4.1.3 was performed, except that peripheral blood mononuclear cells (PBMCs) were substituted for NK92 cells and the cells were not stimulated with IL-12. The results are shown in the table below.

### IFNγ Induction in PBMCs

| Bifunctional Cytokine | EC₅₀ |
|---|---|
| Composition/Cytokine | (nM) |
| Composition 1 | 0.51 |
| Composition 2 | 0.93 |
| Composition 3 | 1.65 |
| Composition 4 | 0.59 |
| Composition 5 | 1.90 |
| SEQ ID NO: 30 | No signal |
| SEQ ID NO: 300 | No signal |
| SEQ ID NO: 30 + SEQ ID | 1.1 |
| NO: 300 | |

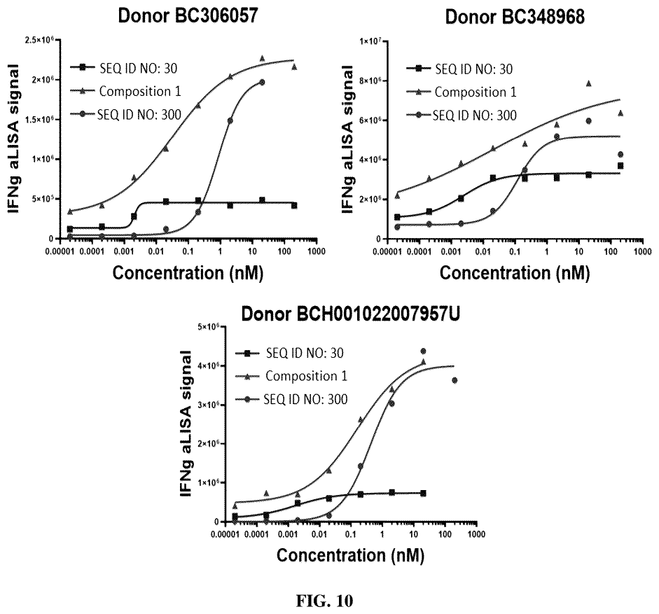

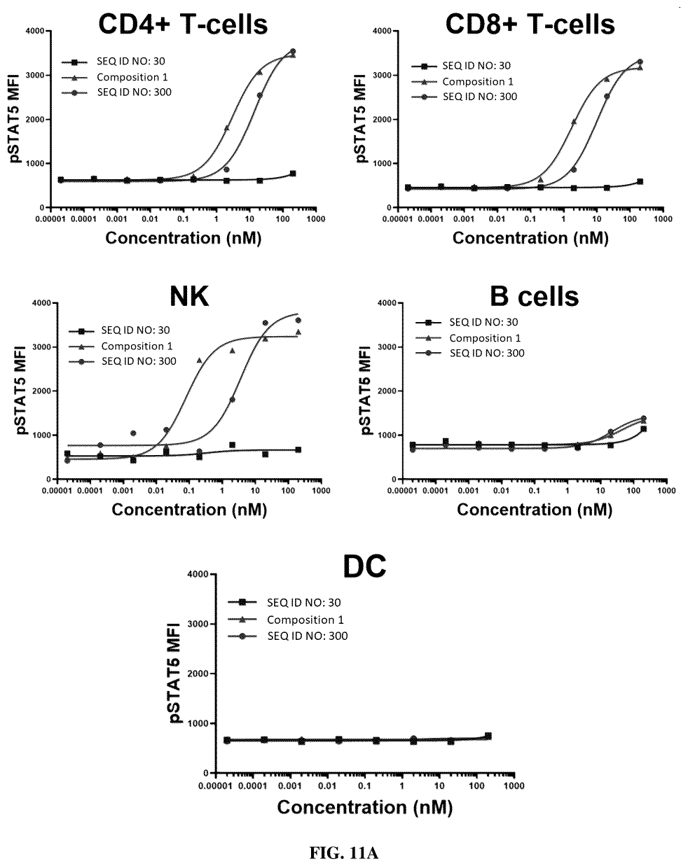

Representative dose response curves for three individual PBMC donors is shown in FIG. 10 for Composition 1 alongside SEQ ID NO: 30 and SEQ ID NO: 300 following 24 hours of stimulation. While SEQ ID NO: 30 had a lower EC50 than SEQ ID NO: 300, the level of IFNγ was substantially lower. However, treatment with the bifunctional cytokine composition resulted in enhanced IFNγ activity compared to SEQ ID NO: 30 achieved at a substantially lower concentration than SEQ ID NO: 300, thus showing a benefit from both cytokines.

### 4.1.4 IL-18 Binding Protein Inhibition Cellular Assay

The NK cell line NK-92 derived from a patient with lymphoma (ATCC® CRL-2407™) is cultured in aMEM medium supplemented with 20% FCS-Glutamax, 25 μM B-mercaptoethanol, and 100 IU/mL of recombinant human IL-2.

On the day of experiment, cells are harvested and washed with aMEM medium without IL-2 and containing 1 ng/mL of recombinant human IL-12. After counting, the cells are seeded at 100,000 cells/well in a 384 well titer plate and incubated at 37° C./5% CO₂. Sixteen 2-fold serial dilutions of Fc-fused human IL-18 binding protein isoform a (IL-18BPa) are prepared in αMEM medium. 1 ng/mL of IL-12 containing 2 nM of each bifunctional cytokine composition is added to the NK-92 cells. The final IL-18 analyte concentration is 1 nM, and the final IL-18BPa concentration ranged from 566 nM to 17 pM.

After incubating the cells for 16-20 hr at 37° C./5% CO₂, 5 μL of the supernatant is carefully transferred to a 384 microwell OptiPlate. IFNγ levels are measured using a human IFNγ AlphaLISA Assay Kit. Briefly, 10 μL of 2.5× AlphaLISA anti-IFNγ acceptor beads and biotinylated antibody anti-IFNγ mix are added to 5 μL of NK-92 supernatants. The mixtures are incubated for 1 hr at room temperature with shaking. Under subdued light, 2.5 μL of 2×SA donor beads are pipetted in each well and incubated for 30 min at room temperature with shaking. AlphaLISA signals are then measured on an EnSpire™ plate reader using 680 nm and 615 nm as excitation and emission wavelengths, respectively. Half maximal inhibitory concentrations (IC₅₀) are calculated based on a variable slope and four parameter analysis using GraphPad PRISM software.

### 4.1.4A IL-18 Binding Protein Inhibition Cellular Assay

An experiment substantially identical to that described above was performed for a variety of IL-18 polypeptide variants, including that of SEQ ID NO: 30, the results of which are shown below along with EC₅₀s for the same IL-18 polypeptide variants as described in the experiment for Example 4.1.3. It is expected that bifunctional cytokine compositions containing the same IL-18 polypeptides as those described below would exhibit similar effects (i.e., similar relative change to EC₅₀s and similar IC₅₀s or superior owing to lesser IL-18BP binding).

### 4.2.1 BLI Binding Assay for IL-2Rβ (CD122) and CD218a (IL-18 Receptor Alpha Subunit)

A biolayer interferometry experiment was performed to compare binding of unconjugated cytokines to bifunctional cytokine compositions to CD122 and CD218a. Results are shown below.

| Bifunctional | | | | |
|---|---|---|---|---|
| Cytokine | | | | |
| Composition/ | | | | |
| Cytokine | Ligand | KD (M) | Ka (1/Ms) | Kd (1/s) |
| SEQ ID NO: 300 | CD122 (avi) | 2.06E−07 | 4.93E+05 | 1.02E−01 |
| Composition 4 | CD122 (avi) | 1.48E−07 | 5.85E+05 | 8.63E−02 |
| Composition 3 | CD122 (avi) | 1.51E−07 | 5.59E+05 | 8.42E−02 |
| SEQ ID NO: 30 | CD218a (avi) | 2.61E−08 | 2.17E+06 | 5.67E−02 |
| Composition 4 | CD218a (avi) | 6.64E−08 | 1.07E+06 | 7.08E−02 |
| Composition 3 | CD218a (avi) | 2.50E−08 | 1.62E+06 | 4.05E−02 |

### 4.2.2 Cell Based In Vitro Characterization of the Bifunctional Cytokine Composition for IL-2 Activity

Experiment are performed to determine the effect of bifunctional composition on human T-cell populations. Primary pan T-cells (CD4+, CD8+ and Tregs T cells) are obtained from healthy donor buffy coat by peripheral blood mononuclear cell (PBMC) purification using ficoll gradient centrifugation followed by negative selection with magnetic beads and then cryopreserved until use. Pan T-cells are thawed, allowed to recover overnight in T-cell medium (RPMI 10% FCS, 1% Glutamin, 1% NEAA, 25 mM bMeoH, 1% NaPyrovate), and after two washing steps with PBS cells, resuspended in PBS. Cells are then distributed at 200,000 cells per well and stimulated with 3.16-fold serial dilutions of aldesleukin or bifunctional cytokine composition with a starting concentration of 316 nM down to 3 pM, for 20 min to 40 min at 37° C./5°/OC02. After incubation, cells are fixed and permeabilized using the Transcription Factor Phospho Buffer kit followed by a surface and intracellular immunostaining for CD4, CD8, CD25, FoxP3, CD45RA and pStat5 to enable cell subset identification and measurement of Stat5 (signal transducer and activator of transcription 5) phosphorylation levels. The FACS (fluorescence activated cell sorting) measurement is done either with a NovoCyte or a Quanteon Flow Cytometer from Acea.

pStat5 MFI (medium fluorescence intensity) signals for the T-cell subsets shown in Table (a) are plotted against concentrations of aldesleukin or of modified IL-2 polypeptides. Half maximal effective concentration (EC₅₀) is calculated based on a variable slope, four parameter analysis using GraphPad PRISM software.

TABLE (a)
Gating Strategy for T-cell Subset Identification

| Cell Type | Selective Markers |
|---|---|
| Treg | CD4⁺, CD25^{Hi}, Foxp3⁺ |
| CD8 Teff | CD8⁺ |
| Naïve CD8 Teff | CD8⁺, CD45RA⁺ |
| CD4 Conventional | CD4⁺, Foxp3⁻ |

Results of an experiment performed substantially similarly to that described above are reported in the table below.

TABLE C
Results of the pSTAT5 assay

| | NK cells | CD4 T cells | CD8 T cells |
|---|---|---|---|
| Bifunctional Cytokine | EC50 | EC50 | EC50 |
| Composition/Cytokine | (nM) | (nM) | (nM) |
| Composition 1 | <0.01 | 0.3588 | 0.097 |
| Composition 2 | <0.01 | 0.2157 | 0.061 |
| Composition 3 | <0.01 | 0.2026 | 0.051 |
| Composition 4 | <0.01 | 0.1769 | 0.047 |
| Composition 5 | n.t. | n.t. | n.t. |
| SEQ ID NO: 300 | <0.01 | 0.2735 | 0.029 |
| SEQ ID NO: 30 | n.d. | n.d. | n.d. |
| SEQ ID NO: 300 + | <0.01 | 0.2068 | 0.042 |
| SEQ ID NO: 30 | | | |

n.t. is not tested;
n.d. is not determined

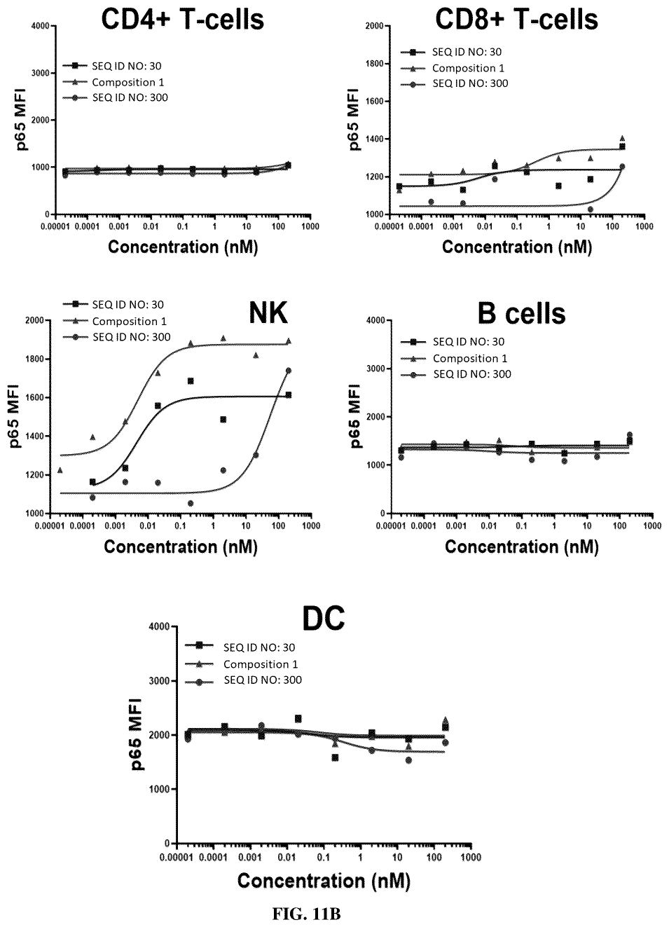

FIG. 11A shows dose response curves of pSTAT5 stimulation for Composition 1 in CD4⁺ T cells, CD8⁺ T cells, natural killer cells (NK), B cells, and dendritic cells (DC). Notably, pSTAT5 EC50s were enhanced in CD4⁺ T cells, CD8⁺ T cells, and NK cells by about 2×, 6×, and 45× respectively as compared to SEQ ID NO: 300 alone. There was no substantial effect on pSTAT5 in B cells or dendritic cells.

An analogous experiment to that described above for pSTAT5 activation was performed for p65 activation using a p65 staining ELISA kit. p65 staining acts as a measurement of IL-18 signaling pathway activation.

### Results of p65 Assay

| | NK cells | CD4 T cells | CD8 T cells |
|---|---|---|---|
| Duokine Composition/ | EC50 | EC50 | EC50 |
| Cytokine | (nM) | (nM) | (nM) |
| Composition 1 | <0.0001 | No signal | 0.001946 |
| Composition 2 | <0.0001 | No signal | <0.0001 |
| Composition 3 | <0.0001 | No signal | <0.0001 |
| Composition 4 | <0.0001 | No signal | <0.0001 |
| Composition 5 | Not tested | Not tested | Not tested |
| SEQ ID NO: 30 | <0.0001 | No signal | <0.0001 |
| SEQ ID NO: 300 | No signal | No signal | No signal |
| SEQ ID NO: 300 + | <0.0001 | No signal | <0.0001 |
| SEQ ID NO: 30 | | | |

FIG. 11B shows dose response curves of pSTAT5 stimulation for Composition 1 in CD4+ T cells, CD8+ T cells, natural killer cells (NK), B cells, and dendritic cells (DC) following 40 minutes of stimulation. Notably, the bifunctional cytokine composition activated p65 more potently than SEQ ID NO: 30 alone in NK cells, while no substantial activity was observed in any of the other cell types.

### 4.2.3—HEK-Blue Cell Based IL18R Reporter Assay

An IL-18R positive HEK-Blue reporter cell line is used to determine binding of IL-18 variants to IL-18R and subsequent downstream signaling. The general protocol is outlined below.

5×104 cells HEK-Blue IL18R reporter cells (InvivoGen, #hkb-hmill8) are seeded into each well of a 96 well plate and stimulated with 0-100 nM of IL-18 polypeptide variants at 37° C. and 5% CO2. After 20 h incubation, 20 μL of cell culture supernatant is then taken from each well and mixed with 180 μL QUANTI-Blue media in a 96 well plate, incubated for 1 hour at 37° C. and 5% CO2. The absorbance signal at 620 nm is then measured on an Enspire plate reader with 680 and 615 nm as excitation and emission wavelengths, respectively. Half Maximal Effective dose (EC50) is calculated based on a variable slope, four parameter analysis using GraphPad PRISM software.

Results of an experiment performed substantially according to the above described protocol for select variants are shown below in Table D, along with results from a corresponding experiment utilizing an IL-2Rbg positive HEK-Blue reporter cell line.

TABLE D
EC₅₀ in HEK-Blue IL18R Reporter Assay Data

| Bifunctional Cytokine | HEK-Blue IL18R | HEK-Blue IL2Rβγ |
|---|---|---|
| Composition/Cytokine | EC50 (pM) | EC50 (nM) |
| Composition 1 | 0.41 | 0.008 |
| Composition 2 | 0.18 | 0.007 |
| Composition 3 | 0.10 | 0.007 |
| Composition 4 | 1.48 | 0.050 |
| Composition 5 | 0.18 | 0.007 |
| SEQ ID NO: 300 | n.d. | 0.004 |
| SEQ ID NO: 30 | 0.067 | n.d. |
| SEQ ID NO: 300 + | n.t. | n.t. |
| SEQ ID NO: 30 | | |

n.t. is not tested; n.d. is not determined.

### Example 5: In Vivo Anti-Tumor Activity

An in vivo efficacy study of IL-2/IL-18 bifunctional cytokine compositions is performed in mice. Naive, 6-8 weeks old, BALB/c female mice (Shanghai SLAC Laboratory Animal Co., LTD, Shanghai, China) are inoculated subcutaneously at the right lower flank with CT26 tumor cells (3×105) in 0.1 mL of PBS for tumor development. The animals are randomized (using an Excel-based randomization software performing stratified randomization based upon tumor volumes), and treatment started when the average tumor volume reached approximately 65 mm³. Animals treated with bifunctional cytokine composition receive two to three 10 mL/kg bolus intravenous (i.v.) injections of 0.3, 1, 3, and 6 mg/kg (18, 61, 184, and 368 nmoles/kg respectively) of bifunctional cytokine composition or control injection. After inoculation, the animals are checked daily for morbidity and mortality. At the time, animals are checked for effects on tumor growth and normal behavior such as mobility, food and water consumption, body weight gain/loss (body weights were measured twice weekly), eye/hair matting and any other abnormal effect. The major endpoints are delayed tumor growth or complete tumor regression. Tumor sizes are measured twice a week in two dimensions using a caliper, and the volume was expressed in mm³ using the formula: V=0.5 a×b2 where a and b are the long and short diameters of the tumor, respectively. Death and observed clinical signs are recorded on the basis of the numbers of animals within each subset.

A re-challenge study is performed on tumor-free animals. Three months after start of treatment, animals that show complete tumor regression are enrolled in a re-challenge study to probe the establishment of a long-lasting immunological memory response. Briefly, ten naive non-treated control animals, one animal previously treated with the bifunctional cytokine composition at 3 mg/kg, five animals previously treated with bifunctional cytokine composition at 6 mg/kg are inoculated subcutaneously at the left lower flank with CT26 tumor cells (3×105) in 0.1 mL of PBS. At the time of routine monitoring, animals are checked for effects on tumor growth and normal behavior such as mobility, food and water consumption, body weight gain/loss (body weights were measured twice weekly), eye/hair matting and any other abnormal effect. The major endpoints are delayed tumor growth or tumor graft rejection. Tumor sizes are measured twice a week in two dimensions using a caliper, and the volume is expressed in mm3 using the formula: V=0.5 a×b2 where a and b are the long and short diameters of the tumor, respectively. Death and observed clinical signs are recorded on the basis of the numbers of animals within each subset. Ninety days after first treatment initiation (69 days after end of treatment) ten naive animals and nine experienced animal that show completed tumor rejection are rechallenged with 3×10⁵ CT26 cells s.c in the opposite flank. Experienced animals are the following: one animal to be treated with bifunctional composition at 3 mg/kg, five animals treated with bifunctional cytokine composition at 6 mg/kg, and three animals to be treated with bifunctional cytokine composition at 3 mg/kg in combination with anti-PD 1 antibody at 10 mg/kg.

It is expected animals treated with bifunctional cytokine compositions will exhibits better anti-tumor response compared to corresponding controls receiving individual cytokines or combinations of cytokines co-administered but not linked in bifunctional cytokine compositions.

### Example 6: Pharmacokinetic/Pharmacodynamic Studies in Non-Human Primates

A pharmacokinetic/pharmacodynamic experiment is performed in cynomolgus monkeys following single intravenous administration. Cynomolgus monkeys of Mauritius origin at least 24-months old are acclimated for 14 days and allocated to 3 groups (1 Male/1 Female per group) and dosed via a 15-minute intravenous infusion of bifunctional cytokine composition at doses of 0.01, 0.03 and 0.1 mg/kg (0.61, 1.83 and 6.1 nmoles/kg respectively) formulated in 10 mM sodium acetate buffer, 8.4% w/v sucrose, 0.02% w/v, polysorbate 80 at pH 5.0. Animals are sampled for blood at various time points for pharmacokinetics and immunophenotyping assessment. Blood samples for pharmacokinetics (500 pL) are collected from the appropriate vein (femoral artery/vein when possible) into a tube containing K2EDTA and placed on wet ice pending centrifugation (10 min, 3500 rpm, +4° C.). Resulting plasma is stored at −80° C. until bioanalysis. Bioanalysis of plasma samples is performed using a sandwich Meso Scale Discovery (MSD) electrochemiluminescence (ECL) assay built on anti-IL-2 (R&D Systems, cat. no. MAB2021) and biotinylated anti-PEG (GenScript, cat. no. A01796-100) antibodies as capture and detection reagents, respectively. PK evaluation is subjected to a non-compartmental pharmacokinetic analysis by using the Phoenix software (version 8.2.0, Certara). The linear/log trapezoidal rule is applied in obtaining the PK parameters.

Blood samples for immunophenotyping (150 pL) are collected and transferred into a 96-well V-bottom plate then fixed and permeabilized with precooled 1×TFP Fix/Perm buffer (Transcription Factor Phospho Buffer Set, BD Biosciences, #563239) during 50-60 minutes at 4° C. Cells are then washed twice with 200 ml of 1×TFP Perm/Wash buffer (Transcription Factor Phospho Buffer Set, BD Biosciences, #563239) at 4° C.). Cells are then permeabilized on ice for 20-22 minutes using 200 ml BD Phosflow Perm Buffer III (BD Biosciences, #558050) precooled at −20° C. After permeabilization, cells are washed three times with 200 ml of 1×TFP Perm/Wash buffer at 4° C. and then stained with the antibody mix during 60-65 min at 4° C. Finally, cells are washed (500 g, 5 min at 4° C. twice), resuspended in 250 pL of flow buffer (stored at 5° C. if needed), and analyzed on the MACSquant flow cytometer and analyzed with the MACSQuantify software (Miltenyi Biotec).

It is expected that bifunctional cytokine compositions will exhibit better PK and PD profiles compared to the cytokines alone.

### Example 7: Characteristics of Additional IL-18 Polypeptides

7A—HEK-Blue Reporter Assay—The HEK-Blue IL-18R reporter assay described above was also performed on additional IL-18 polypeptides which can be incorporated into bifunctional cytokine compositions (e.g., IL-2/IL-18 bifunctional cytokine compositions) as provided herein. It is expected that the IL-18 polypeptides provided below would behave similarly to C086 (SEQ ID NO: 30) when incorporated into a bifunctional cytokine composition as those otherwise provided herein.

| SEQ ID NO: or | | |
|---|---|---|
| Composition ID | Sequence modifications | EC₅₀ (pM) |
| 1 | Native sequence | 3.33 |
| 34 | E6K, K53A, S55A | 272.5 |
| 39 | E6K, K53A | 0.72 |
| 42 | E6K, K53A, S55A, T63A | 0.79 |
| 50 | E6K, K53A, T63A | 1.77 |
| 54 | E6K, C38S, K53A, C68S, K70C, C76S, C127S | 9.12 |
| 56 | E6K, K53A, C38S, C76S, C127S | 3.73 |
| 57 | E6K, C38S, K53A | 0.86 |
| 30 | E6K, V11I, C38A, K53A, T63A, C76A, C127A | 0.034 |
| 62 | E6K, C38A, K53A, C127A | 0.17 |
| 60 | E6K, C38Q, K53A | 0.203 |
| 59 | E6K, C38A, K53A | 0.268 |
| 57 | E6K, C38S, K53A | 0.53 |
| C143 | V11I, C38A, K53A, C76A, C127A | 0.98 |
| C144 | V11I, C38A, K53A, T63A, C76A, C127A | 0.17 |
| C145 | V11I, C38A, K53A, S55A, C76A, C127A | 3.63 |
| C146 | V11I, C38A, M51G, K53A, C76A, C127A | 0.8 |
| C147 | V11I, C38A, K53A, D54A, C76A, C127A | 1 |
| C148 | F2A, V11I, C38A, K53A, C76A, C127A | 7.28 |
| C149 | V11I, E31A, C38A, K53A, C76A, C127A | 6.6 |
| C150 | V11I, T34A, C38A, K53A, C76A, C127A | 0.7 |
| C151 | V11I, D35A, C38A, K53A, C76A, C127A | 13.12 |
| C152 | V11I, S36A, C38A, K53A, C76A, C127A | 0.25 |
| C153 | V11I, D37A, C38A, K53A, C76A, C127A | 14.12 |
| C154 | V11I, E31A, D37A, C38A, K53A, C76A, C127A | 11.95 |
| C155 | V11I, C38A, D40A, K53A, C76A, C127A | 0.52 |
| C156 | V11I, C38A, N41A, K53A, C76A, C127A | 11.7 |
| C157 | V11I, C38A, K53A, C76A, C127A, D132A | 1.95 |
| C158 | V11I, C38A, K53A, C76A, G108A, C127A | 15.56 |
| C159 | V11I, C38A, K53A, C76A, H109A, C127A | 19.5 |
| C160 | V11I, C38A, K53A, C76A, D110A, C127A | 2.02 |
| C161 | K8R, V11I, C38A, C76A, Q103E, C127A | 2.01 |
| C162 | K8E, V11I, C38A, C76A, Q103R, C127A | 2.3 |
| C163 | V11I, C38A, C76A, Q103K, C127A | 1.5 |
| C164 | V11I, C38A, S55H, C76A, C127A | 3.14 |
| C165 | V11I, C38A, S55R, C76A, C127A | 1.91 |
| C166 | V11I, C38A, S55T, C76A, C127A | 4.73 |
| C167 | V11I, C38A, C76A, S105I, C127A | 5.37 |
| C168 | V11I, C38A, C76A, S105K, C127A | 7.73 |
| C174 | K8L, E6K, V11I, C38A, K53A, T63A, C76A, C127A | 0.29 |
| C176 | E6K, V11I, C38A, I49M, K53A, T63A, C76A, C127A | 0.07 |
| C177 | E6K, V11I, C38A, I49R, K53A, T63A, C76A, C127A | 0.04 |
| C178 | E6K, V11I, C38A, K53A, T63A, C76A, Q103R, C127A | 0.26 |
| C179 | E6K, K8E, V11I, C38A, K53A, T63A, C76A, Q103R, | 0.4 |
| | C127A | |
| C181 | E6K, V11I, C38A, K53A, T63A, C76A, C127A, V153E | 0.1 |
| C182 | E6K, V11I, C38A, K53A, T63A, C76A, C127A, V153Y | 0.08 |
| C183 | E6K, V11I, C38A, M51G, K53A, T63A, C76A, C127A | 0.1 |
| C184 | E6R, V11I, C38A, K53A, T63A, C76A, C127A | 0.04 |
| C140 | E6K, V11I, C38A, K53A, T63A, C76A, C127A | 2.5 |
| C141 | E6K, V11I, C38A, K53A, T63A, C76A, C127A | 1.68 |
| C142 | Y1M, E6K, V11I, C38A, K53A, T63A, C76A, C127A | 0.02 |
| C192 | E6K, V11I, C38A, M51G, K53A, T63A, C76A, C127A | 13.99 |

7B—IL-18 BP AiphaLISA assay—An IL-18 binding protein AiphaLISA experiment substantially as described in Example 4.1.2 was performed on IL-18 polypeptide which can be incorporated into bifunctional cytokine compositions (e.g., IL-2/IL-18 bifunctional cytokine compositions) as provided herein to assess ability to bind to IL-18BP. Results are shown in the Table below.

| SEQ ID NO: or | | |
|---|---|---|
| Composition ID | Sequence modifications | KD (nM) |
| 1 | Native Sequence | 0.67 |
| 34 | E06K, K53A, S55A | >1500 |
| 35 | Y01G, F02A, E06K, M51G, K53A, D54A, S55A, T63A | 969.0 |
| 36 | K53A | 513.8 |
| 37 | S55A | 10.7 |
| 38 | E06K | 0.13 |
| 39 | E06K, K53A | 130.3 |
| 40 | E06K, S55A | 12.3 |
| 41 | K53A, S55A | 500.0 |
| 42 | E06K, K53A, S55A, T63A | 822.0 |
| 43 | E06K, K53A, S55A, Y01G | |
| 44 | E06K, K53A, S55A, F02A | >1000 |
| 45 | E06K, K53A, S55A, D54A | >1000 |
| 46 | E06K, K53A, S55A, M51G | >1000 |
| 47 | C38S, C68S, C76S, C127S | 0.03 |
| 48 | C38S, C68S, C76S, C127S, K70C | 0.21 |
| 49 | E06K, K53A, S55A, C38S, C68S, C76S, C127S, K70C | >1000 |
| 50 | E06K, K53A, T63A | 339.8 |
| 51 | T63A | 2.59 |
| 52 | E06K, T63A | 0.83 |
| 53 | K53A, T63A | 198 |
| 54 | E06K, K53A, C38S, C68S, C76S, C127S, K70C | 446.0 |
| 55 | K53A, T63A, C38S, C68S, C76S, C127S, K70C | 913 |
| 56 | E6K, K53A, C38S, C76S, C127S | 435.5 |
| 57 | E6K, K53A, C38S | 50.2 |
| C143 | V11I, C38A, K53A, C76A, C127A | 8.86 |
| C144 | V11I, C38A, K53A, T63A, C76A, C127A | 0.66 |
| C145 | V11I, C38A, K53A, S55A, C76A, C127A | 9.74 |
| C146 | V11I, C38A, M51G, K53A, C76A, C127A | 373.30 |
| C147 | V11I, C38A, K53A, D54A, C76A, C127A | 25.77 |
| C148 | F2A, V11I, C38A, K53A, C76A, C127A | 57.21 |
| C149 | V11I, E31A, C38A, K53A, C76A, C127A | 0.64 |
| C150 | V11I, T34A, C38A, K53A, C76A, C127A | 1.24 |
| C151 | V11I, D35A, C38A, K53A, C76A, C127A | 2.88 |
| C152 | V11I, S36A, C38A, K53A, C76A, C127A | 1.12 |
| C153 | V11I, D37A, C38A, K53A, C76A, C127A | 4.55 |
| C154 | V11I, E31A, D37A, C38A, K53A, C76A, C127A | 2.12 |
| C155 | V11I, C38A, D40A, K53A, C76A, C127A | 0.74 |
| C156 | V11I, C38A, N41A, K53A, C76A, C127A | 18.47 |
| C157 | V11I, C38A, K53A, C76A, C127A, D132A | 13.70 |
| C158 | V11I, C38A, K53A, C76A, G108A, C127A | 1.24 |
| C159 | V11I, C38A, K53A, C76A, H109A, C127A | 0.55 |
| C160 | V11I, C38A, K53A, C76A, D110A, C127A | 0.71 |
| C161 | K8R, V11I, C38A, C76A, Q103E, C127A | 0.06 |
| C162 | K8E, V11I, C38A, C76A, Q103R, C127A | 0.85 |
| C163 | V11I, C38A, C76A, Q103K, C127A | 0.05 |
| C164 | V11I, C38A, S55H, C76A, C127A | 0.08 |
| C165 | V11I, C38A, S55R, C76A, C127A | 0.15 |
| C166 | V11I, C38A, S55T, C76A, C127A | 0.02 |
| C167 | V11I, C38A, C76A, S105I, C127A | 0.04 |
| C168 | V11I, C38A, C76A, S105K, C127A | 0.05 |
| C174 | K8L, E6K, V11I, C38A, K53A, T63A, C76A, C127A | 0.14 |
| C176 | E6K, V11I, C38A, I49M, K53A, T63A, C76A, C127A | 25.84 |
| C177 | E6K, V11I, C38A, I49R, K53A, T63A, C76A, C127A | >2800 |
| C178 | E6K, V11I, C38A, K53A, T63A, C76A, Q103R, C127A | >2800 |
| C179 | E6K, K8E, V11I, C38A, K53A, T63A, C76A, Q103R, | >2800 |
| | C127A | |
| C180 | E6K, V11I, C38A, K53A, T63A, C76A, C127A, V153R | |
| C181 | E6K, V11I, C38A, K53A, T63A, C76A, C127A, V153E | >2800 |
| C182 | E6K, V11I, C38A, K53A, T63A, C76A, C127A, V153Y | >2800 |
| C183 | E6K, V11I, C38A, M51G, K53A, T63A, C76A, C127A | >2800 |
| C184 | E6R, V11I, C38A, K53A, T63A, C76A, C127A | 5.46 |
| C140 | E6K, V11I, C38A, K53A, T63A, C76A, C127A | >2800 |
| C141 | E6K, V11I, C38A, K53A, T63A, C76A, C127A | >2800 |
| C142 | Y1M, E6K, V11I, C38A, K53A, T63A, C76A, C127A | 2.25 |
| C192 | E6K, V11I, C38A, M51G, K53A, T63A, C76A, C127A | >2800 |

7C—IFNγ Stimulation and IL-18BP Inhibition Assay—The experiments described in Examples 4.1.3 and 4.1.4 were performed substantially as described on modified IL-18 polypeptides in order to assess their activities and their suitability for incorporation into bifunctional cytokine compositions (e.g., IL-2/IL-18 bifunctional cytokine compositions). Results are shown in the table below.

| SEQ ID NO: or | | IC₅₀ | EC₅₀ |
|---|---|---|---|
| Composition ID | Sequence modifications | (nM) | (nM) |
| 1 | Native sequence | 1.47 | 0.276 |
| 34 | E06K, K53A, S55A | 229 | 0.824 |
| 35 | Y01G, F02A, E06K, M51G, K53A, D54A, S55A, T63A | >55.0 | >55.0 |
| 36 | K53A | 27.3 | 0.444 |
| 37 | S55A | 4.46 | 0.108 |
| 38 | E06K | 7.79 | 0.0567 |
| 39 | E06K, K53A | >703 | 0.0192 |
| 40 | E06K, S55A | 15 | 0.067 |
| 41 | K53A, S55A | 37.3 | 1.58 |
| 42 | E06K, K53A, S55A, T63A | 1060 | 0.144 |
| 43 | E06K, K53A, S55A, Y01G | 27.8 | 6.12 |
| 44 | E06K, K53A, S55A, F02A | NT | >1000 |
| 45 | E06K, K53A, S55A, D54A | NT | 30 |
| 46 | E06K, K53A, S55A, M51G | 0.189 | 7.4 |
| 47 | C38S, C68S, C76S, C127S | 0.444 | 0.115 |
| 48 | C38S, C68S, C76S, C127S, K70C | 0.114 | 0.488 |
| 49 | E06K, K53A, S55A, C38S, C68S, C76S, C127S, K70C | NT | 58.5 |
| 50 | E06K, K53A, T63A | >1000 | 0.0268 |
| 51 | T63A | 0.239 | 0.449 |
| 52 | E06K, T63A | 47.1 | 0.011 |
| 53 | K53A, T63A | 18.2 | 0.155 |
| 54 | E06K, K53A, C38S, C68S, C76S, C127S, K70C | 23.5 | 0.962 |
| 55 | K53A, T63A, C38S, C68S, C76S, C127S, K70C | >1000 | 17.2 |
| 6 | E6K, V11I, C38A, K53A, T63A, C68A, C76A, C127A, | 5.847 | 1.366 |
| | D98C | | |
| 5 | E6K, V11I, C38A, K53A, T63A, C68A, C76A, C127A, | 62.37 | 0.075 |
| | M86C | | |
| 9 | E6K, C38A, K53A, C68A, D98C | 960.8 | 0.069 |
| 4 | E6K, C38A, K53A, C68A, M86C | 396.3 | 0.022 |
| 30 | E6K, V11I, C38A, K53A, T63A, C76A, C127A | 283.6 | 0.026 |
| 62 | E6K, C38A, K53A, C127A | 780.5 | 0.006 |
| 60 | E6K, C38Q, K53A | 653.5 | 0.015 |
| 59 | E6K, C38A, K53A | 146.2 | 0.045 |
| 57 | E6K, C38S, K53A | 1.625 | 0.138 |
| C143 | V11I, C38A, K53A, C76A, C127A | 7.522 | 0.012 |
| C144 | V11I, C38A, K53A, T63A, C76A, C127A | 10.24 | 0.087 |
| C145 | V11I, C38A, K53A, S55A, C76A, C127A | 732.9 | 0.037 |
| C146 | V11I, C38A, M51G, K53A, C76A, C127A | 47.63 | 0.079 |
| C147 | V11I, C38A, K53A, D54A, C76A, C127A | 5.055 | 0.256 |
| C148 | F2A, V11I, C38A, K53A, C76A, C127A | 1.167 | 0.187 |
| C149 | V11I, E31A, C38A, K53A, C76A, C127A | 21.27 | 0.015 |
| C150 | V11I, T34A, C38A, K53A, C76A, C127A | 3.622 | 0.061 |
| C151 | V11I, D35A, C38A, K53A, C76A, C127A | 7.85 | 0.033 |
| C152 | V11I, S36A, C38A, K53A, C76A, C127A | 2.222 | 0.175 |
| C153 | V11I, D37A, C38A, K53A, C76A, C127A | 3.709 | 0.062 |
| C154 | V11I, E31A, D37A, C38A, K53A, C76A, C127A | 3.233 | 0.067 |
| C155 | V11I, C38A, D40A, K53A, C76A, C127A | 0.681 | 0.558 |
| C156 | V11I, C38A, N41A, K53A, C76A, C127A | 6.082 | 0.056 |
| C157 | V11I, C38A, K53A, C76A, C127A, D132A | 3.981 | 0.073 |
| C158 | V11I, C38A, K53A, C76A, G108A, C127A | 1.807 | 0.123 |
| C159 | V11I, C38A, K53A, C76A, H109A, C127A | 3.181 | 0.028 |
| C160 | V11I, C38A, K53A, C76A, D110A, C127A | 1.073 | 0.057 |
| C161 | K8R, V11I, C38A, C76A, Q103E, C127A | 7.292 | 0.061 |
| C162 | K8E, V11I, C38A, C76A, Q103R, C127A | 0.823 | 0.093 |
| C163 | V11I, C38A, C76A, Q103K, C127A | 0.456 | 0.414 |
| C164 | V11I, C38A, S55H, C76A, C127A | 0.885 | 0.176 |
| C165 | V11I, C38A, S55R, C76A, C127A | 0.44 | 0.098 |
| C166 | V11I, C38A, S55T, C76A, C127A | 0.809 | 0.103 |
| C167 | V11I, C38A, C76A, S105I, C127A | 0.176 | 0.098 |
| C168 | V11I, C38A, C76A, S105K, C127A | 5.847 | 1.366 |

Although the present disclosure and its advantages have been described in detail, it should be understood that various changes, substitutions and alterations can be made herein without departing from the spirit and scope of the disclosure as defined in the appended claims.

1. A bifunctional cytokine composition, comprising:
a first cytokine;
a second cytokine; and
a chemical linker comprising a first point of attachment to the first cytokine and a second point of attachment to the second cytokine.

2-8. (canceled)

9. The bifunctional cytokine composition of claim 1, wherein the chemical linker comprises polyethylene glycol.

10-16. (canceled)

17. The bifunctional cytokine composition of claim 1, wherein the first cytokine is an IL-18.

18. The bifunctional cytokine composition of claim 17, wherein the second cytokine is an IL-2, an IL-12, or an IL-7.

19-36. (canceled)

37. A bifunctional cytokine composition, comprising:
an interleukin-18 (IL-18) polypeptide, wherein residue position numbering of the IL-18 polypeptide is based on SEQ ID NO: 1 as a reference sequence;
an interleukin-2 (IL-2) polypeptide, wherein the IL-2 polypeptide is biased towards the IL-2 receptor subunit beta (IL-2Rβ) compared to wild type IL-2, wherein residue position numbering of the IL-2 polypeptide is based on SEQ ID NO: 301 as a reference sequence; and
a linker comprising a first point of attachment to the IL-18 polypeptide and a second point of attachment to the IL-2 polypeptide.

38. The bifunctional cytokine composition of claim 37, wherein the IL-2 polypeptide exhibits reduced binding to the IL-2 receptor subunit alpha (IL-2Rα).

39. (canceled)

40. The bifunctional cytokine composition of claim 37, wherein the IL-2 polypeptide comprises an amino acid substitution at residue 35, 37, 38, 41, 42, 43, 44, 45, 60, 61, 62, 64, 65, 68, 69, 71, 72, 104, 105, 107, or any combination thereof, of the IL-2 polypeptide, wherein residue position numbering is based on SEQ ID NO: 301 as a reference sequence.

41. (canceled)

42. The bifunctional cytokine composition of claim 37, wherein the IL-2 polypeptide comprises a non-linker polymer attached at residue 42 or 45, or two non-linker polymers, wherein one of the two non-linker polymers is attached at residue 42 and one of the two non-linker polymers is attached at residue 45.

43-44. (canceled)

45. The bifunctional cytokine composition of claim 37, wherein the IL-2 polypeptide comprises an amino acid sequence having at least about 80%, 85%, 90%, 95%, 96%, 97%, 98%, 99%, or identical to a sequence set forth in SEQ ID NOs: 303.

46. (canceled)

47. The bifunctional cytokine composition of claim 37, wherein the second point of attachment is at residue 1, 42, or 45 of the IL-2 polypeptide.

48. The bifunctional cytokine composition of claim 37, wherein the bifunctional cytokine composition exhibits reduced binding to IL-18 binding protein (IL-18BP) compared to wild type IL-18 (WT IL-18).

49. (canceled)

50. The bifunctional cytokine composition of claim 37, wherein the IL-18 polypeptide comprises a Y01G, F02A, E06K, V11I, C38S, C38A, K53A, D54A, S55A, T63A, C76S, C76A, E85C, M86C, T95C, D98C, C127S, or C127A amino acid substitution, or any combination thereof.

51. The bifunctional cytokine composition of claim 37, wherein the IL-18 polypeptide comprises an amino acid sequence having at least about 80%, 85%, 90%, 95%, 96%, 97%, 98%, 99%, or identical to a sequence set forth in SEQ ID NOs: 30.

52-56. (canceled)

57. The bifunctional cytokine composition of claim 37, wherein the first point of attachment is to residue 68 the IL-18 polypeptide.

58. The bifunctional cytokine composition of claim 37, wherein the IL-2 polypeptide comprise the sequence set forth in SEQ ID NO: 303.

59. The bifunctional cytokine composition of claim 37, wherein the bifunctional cytokine composition induces STAT5 phosphorylation in CD4⁺ T cells, CD8⁺ T cells, and/or NK cells more potently than a corresponding IL-2 polypeptide.

60. The bifunctional cytokine composition of claim 37, wherein the bifunctional cytokine composition increases levels of p65 phosphorylation in NK cells more potently than a corresponding IL-18 polypeptide.

61. (canceled)

62. The bifunctional cytokine composition of claim 37, wherein the bifunctional cytokine composition exhibits an EC₅₀ of IFNγ production which is lower than for a corresponding IL-2 polypeptide and/or IL-18 polypeptide.

63. (canceled)

64. The bifunctional cytokine composition of claim 37, wherein the linker comprises from about 2 to about 100 ethylene glycol units.

65-74. (canceled)

75. A method of making a bifunctional cytokine composition, comprising,
a) providing a first cytokine, wherein the first cytokine comprises a first cytokine conjugation handle;
b) providing a second cytokine, wherein the second cytokine comprises a second cytokine conjugation handle;
c) providing a bifunctional reagent, wherein the bifunctional reagent comprises a first reagent conjugation handle and a second reagent conjugation handle,
wherein the first reagent conjugation handle is complementary to the first cytokine conjugation handle, and
wherein the second reagent conjugation handle is complementary to the second cytokine conjugation handle;
d) forming a first covalent bond through a reaction of the first cytokine conjugation handle and the first reagent conjugation handle; and forming a second covalent bond through a reaction of the second cytokine conjugation handle and the second reagent conjugation handle.


SEQUENCE LISTING (330 sequences)

SEQ ID NO: 1 - protein, 157 aa, Homo sapiens
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 2 - protein, 157 aa, synthetic construct
YFGKLKSKLSVIRNLNDQVLFIDQGNRPLFEDMTDSDARDNAPRTIFIISMYADSQPRGMAVTISVKAEKISTLSCENKIISFKCMNPPDNIKDTKSDIIFFQRSVPGHDNKMQFESSSYEGYFLACEKERDLFKLILKKEDELGDRSIMFTVQNED

SEQ ID NO: 3 - protein, 157 aa, synthetic construct
YFGKLKSKLSIIRNLNDQVLFIDQGNRPLFEDMTDSDARDNAPRTIFIISMYADSQPRGMAVAISVKAEKISTLSAENKIISFKCMNPPDNIKDTKSDIIFFQRSVPGHDNKMQFESSSYEGYFLAAEKERDLFKLILKKEDELGDRSIMFTVQNED

SEQ ID NO: 4 - protein, 157 aa, synthetic construct
YFGKLKSKLSVIRNLNDQVLFIDQGNRPLFEDMTDSDARDNAPRTIFIISMYADSQPRGMAVTISVKAEKISTLSCENKIISFKECNPPDNIKDTKSDIIFFQRSVPGHDNKMQFESSSYEGYFLACEKERDLFKLILKKEDELGDRSIMFTVQNED

SEQ ID NO: 5 - protein, 157 aa, synthetic construct
YFGKLKSKLSIIRNLNDQVLFIDQGNRPLFEDMTDSDARDNAPRTIFIISMYADSQPRGMAVAISVKAEKISTLSAENKIISFKECNPPDNIKDTKSDIIFFQRSVPGHDNKMQFESSSYEGYFLAAEKERDLFKLILKKEDELGDRSIMFTVQNED

SEQ ID NO: 6 - protein, 157 aa, synthetic construct
YFGKLKSKLSIIRNLNDQVLFIDQGNRPLFEDMTDSDARDNAPRTIFIISMYADSQPRGMAVAISVKAEKISTLSAENKIISFKEMNPPDNIKDTKSCIIFFQRSVPGHDNKMQFESSSYEGYFLAAEKERDLFKLILKKEDELGDRSIMFTVQNED

SEQ ID NO: 7 - protein, 157 aa, synthetic construct
YFGKLKSKLSVIRNLNDQVLFIDQGNRPLFEDMTDSDARDNAPRTIFIISMYADSQPRGMAVTISVKAEKISTLSAENKIISFKECNPPDNIKDTKSDIIFFQRSVPGHDNKMQFESSSYEGYFLAAEKERDLFKLILKKEDELGDRSIMFTVQNED

SEQ ID NO: 8 - protein, 157 aa, synthetic construct
YFGKLKSKLSIIRNLNDQVLFIDQGNRPLFEDMTDSDARDNAPRTIFIISMYADSQPRGMAVAISVKAEKISTLSAENKIISFKEMNPPDNIKDCKSDIIFFQRSVPGHDNKMQFESSSYEGYFLAAEKERDLFKLILKKEDELGDRSIMFTVQNED

SEQ ID NO: 9 - protein, 157 aa, synthetic construct
YFGKLKSKLSVIRNLNDQVLFIDQGNRPLFEDMTDSDARDNAPRTIFIISMYADSQPRGMAVTISVKAEKISTLSCENKIISFKEMNPPDNIKDTKSCIIFFQRSVPGHDNKMQFESSSYEGYFLACEKERDLFKLILKKEDELGDRSIMFTVQNED

SEQ ID NO: 10 - protein, 157 aa, synthetic construct
YFGKLKSKLSVIRNLNDQVLFIDQGNRPLFEDMTDSDARDNAPRTIFIISMYADSQPRGMAVTISVKAEKISTLSAENKIISFKEMNPPDNIKDTKSCIIFFQRSVPGHDNKMQFESSSYEGYFLAAEKERDLFKLILKKEDELGDRSIMFTVQNED

SEQ ID NO: 11 - protein, 157 aa, synthetic construct
YFGKLKSKLSIIRNLNDQVLFIDQGNRPLFEDMTDSDARDNAPRTIFIISMYADSQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 12 - protein, 157 aa, synthetic construct
YFGKLKSKLSVIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 13 - protein, 157 aa, synthetic construct
YFGKLKSKLSVIRNLNDQVLFIDQGNRPLFEDMTDSDCRDNAPRTIFIISMYADSQPRGMAVNISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 14 - protein, 157 aa, synthetic construct
YFGKLKSKLSVIRNLNDQVLFIDQGNRPLFEDMTDSDCRDNAPRTIFIIAMYADSQPRGMAVNISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 15 - protein, 157 aa, synthetic construct
YFGKLKSKLSVIRNLNDQVLFIDQGNRPLFEDMTDSDCRDNAPRTIFIIHMYADSQPRGMAVNISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 16 - protein, 157 aa, synthetic construct
YFGKLKSKLSVIRNLNDQVLFIDQGNRPLFEDMTDSDCRDNAPRTIFIISMYADSQPRGMAVNIAVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 17 - protein, 157 aa, synthetic construct
YFGKLKSKLSVIRNLNDQVLFIDQGNRPLFEDMTDSDCRDNAPRTIFIIHMYADSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 18 - protein, 157 aa, synthetic construct
YFGKLKSKLSVIRNLNDQVLFIDQGNRPLFEDMTDSDARDNAPRTIFIISMYADSQPRGMAVTISVKAEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 19 - protein, 157 aa, synthetic construct
YFGKLKSKLSVIRNLNDQVLFIDQGNRPLFEDMTDSDCRDNAPRTIFIISMYADSQPRGMAVTISVKCEKISTLSCENAIISFKEMNPPDNIKDTKSDIIFFQRSVPGHDNKMQFESSSYEGYFLACEKERDLFKLILKKEDELGDRSIMFTVQNED

SEQ ID NO: 20 - protein, 157 aa, synthetic construct; identical to SEQ ID NO: 19

SEQ ID NO: 21 - protein, 157 aa, synthetic construct
YFGKLKSKLSVIRNLNDQVLFIDQGNRPLFEDMTDSDCRDNAPRTIFIISMYADSQPRGMAVTISVKCEKISTLSCENKIISFKEMNPPDNIKDTKSDIIFFQRSVPAHDNKMQFESSSYEGYFLACEKERDLFKLILKKEDELGDRSIMFTVQNED

SEQ ID NO: 22 - protein, 157 aa, synthetic construct
YFGKLKSKLSVIRNLNDQVLFIDQGNRPLFEDMTDSDCRDNAPRTIFIISMYADSQPRGMAVTISVKCEKISTLSCENKIISFKEMNPPDNIKDTKSDIIFFQRSVPGADNKMQFESSSYEGYFLACEKERDLFKLILKKEDELGDRSIMFTVQNED

SEQ ID NO: 23 - protein, 157 aa, synthetic construct
YFGKLKSKLSVIRNLNDQVLFIDQGNRPLFEDMTDSDCRDNAPRTIFIISMYADSQPRGMAVTISVKCEKISTLSCENKIISFKEMNPPDNIKDTKSDIIFFQRSVPGHDNAMQFESSSYEGYFLACEKERDLFKLILKKEDELGDRSIMFTVQNED

SEQ ID NO: 24 - protein, 157 aa, synthetic construct
YFGKLKSKLSVIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLACEKERDLFKLILKKEDELGDRSIMFTVQNED

SEQ ID NO: 25 - protein, 157 aa, synthetic construct
YFGKLKSKLSVIRNLNDQVLFIDQGNRPLFEDMTDSDQRDNAPRTIFIISMYADSQPRGMAVAISVKCEKISTLSAENKIISFKEMNPPDNIKDTKSDIIFFQRSVPGHDNKMQFESSSYEGYFLACEKERDLFKLILKKEDELGDRSIMFTVQNED

SEQ ID NO: 26 - protein, 157 aa, synthetic construct
YFGKLKSKLSVIRNLNDQVLFIDQGNRPLFEDMTDSDARDNAPRTIFIISMYADSQPRGMAVAISVKCEKISTLSCENKIISFKEMNPPDNIKDTKSDIIFFQRSVPGHDNKMQFESSSYEGYFLAAEKERDLFKLILKKEDELGDRSIMFTVQNED

SEQ ID NO: 27 - protein, 157 aa, synthetic construct
YFGKLKSKLSVIRNLNDQVLFIDQGNRPLFEDMTDSDQRDNAPRTIFIISMYADSQPRGMAVAISVKCEKISTLSCENKIISFKEMNPPDNIKDTKSDIIFFQRSVPGHDNKMQFESSSYEGYFLAAEKERDLFKLILKKEDELGDRSIMFTVQNED

SEQ ID NO: 28 - protein, 157 aa, synthetic construct; identical to SEQ ID NO: 12

SEQ ID NO: 29 - protein, 157 aa, synthetic construct
YFGKLKSKLSIIRNLNDQVLFIDQGNRPLFEDMTDSDARDNAPRTIFIISMYADSQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 30 - protein, 157 aa, synthetic construct
YFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 31 - protein, 157 aa, synthetic construct
YFGKLESKLSVIRNLNDQVLFIDQGNRPLFEDMTDSDARDNAPRTIFIISMYKDSQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 32 - protein, 157 aa, synthetic construct
YFGKLESKLSVIRNLNDQVLFIDQGNRPLFEDMTDSDARDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 33 - protein, 157 aa, synthetic construct
YFGKLKSKLSVIRNLNDQVLFIDQGNRPLFEDMTDSDARDNAPRTIFIISMYADSQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 34 - protein, 157 aa, synthetic construct
YFGKLKSKLSVIRNLNDQVLFIDQGNRPLFEDMTDSDCRDNAPRTIFIISMYADAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 35 - protein, 157 aa, synthetic construct
GAGKLKSKLSVIRNLNDQVLFIDQGNRPLFEDMTDSDCRDNAPRTIFIISGYAAAQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 36 - protein, 157 aa, synthetic construct
YFGKLESKLSVIRNLNDQVLFIDQGNRPLFEDMTDSDCRDNAPRTIFIISMYADSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 37 - protein, 157 aa, synthetic construct
YFGKLESKLSVIRNLNDQVLFIDQGNRPLFEDMTDSDCRDNAPRTIFIISMYKDAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 38 - protein, 157 aa, synthetic construct
YFGKLKSKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 39 - protein, 157 aa, synthetic construct
YFGKLKSKLSVIRNLNDQVLFIDQGNRPLFEDMTDSDCRDNAPRTIFIISMYADSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 40 - protein, 157 aa, synthetic construct
YFGKLKSKLSVIRNLNDQVLFIDQGNRPLFEDMTDSDCRDNAPRTIFIISMYKDAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 41 - protein, 157 aa, synthetic construct
YFGKLESKLSVIRNLNDQVLFIDQGNRPLFEDMTDSDCRDNAPRTIFIISMYADAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 42 - protein, 157 aa, synthetic construct
YFGKLKSKLSVIRNLNDQVLFIDQGNRPLFEDMTDSDCRDNAPRTIFIISMYADAQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 43 - protein, 157 aa, synthetic construct
GFGKLKSKLSVIRNLNDQVLFIDQGNRPLFEDMTDSDCRDNAPRTIFIISMYADAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 44 - protein, 157 aa, synthetic construct
YAGKLKSKLSVIRNLNDQVLFIDQGNRPLFEDMTDSDCRDNAPRTIFIISMYADAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 45 - protein, 157 aa, synthetic construct
YFGKLKSKLSVIRNLNDQVLFIDQGNRPLFEDMTDSDCRDNAPRTIFIISMYAAAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 46 - protein, 157 aa, synthetic construct
YFGKLKSKLSVIRNLNDQVLFIDQGNRPLFEDMTDSDCRDNAPRTIFIISGYADAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 47 - protein, 157 aa, synthetic construct
YFGKLESKLSVIRNLNDQVLFIDQGNRPLFEDMTDSDSRDNAPRTIFIISMYKDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 48 - protein, 157 aa, synthetic construct
YFGKLESKLSVIRNLNDQVLFIDQGNRPLFEDMTDSDSRDNAPRTIFIISMYKDSQPRGMAVTISVKSECISTLSSENKIISFKEMNPPDNIKDTKSDIIFFQRSVPGHDNKMQFESSSYEGYFLASEKERDLFKLILKKEDELGDRSIMFTVQNED

SEQ ID NO: 49 - protein, 157 aa, synthetic construct
YFGKLKSKLSVIRNLNDQVLFIDQGNRPLFEDMTDSDSRDNAPRTIFIISMYADAQPRGMAVTISVKSECISTLSSENKIISFKEMNPPDNIKDTKSDIIFFQRSVPGHDNKMQFESSSYEGYFLASEKERDLFKLILKKEDELGDRSIMFTVQNED

SEQ ID NO: 50 - protein, 157 aa, synthetic construct
YFGKLKSKLSVIRNLNDQVLFIDQGNRPLFEDMTDSDCRDNAPRTIFIISMYADSQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 51 - protein, 157 aa, synthetic construct
YFGKLESKLSVIRNLNDQVLFIDQGNRPLFEDMTDSDCRDNAPRTIFIISMYKDSQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 52 - protein, 157 aa, synthetic construct
YFGKLKSKLSVIRNLNDQVLFIDQGNRPLFEDMTDSDCRDNAPRTIFIISMYKDSQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 53 - protein, 157 aa, synthetic construct
YFGKLESKLSVIRNLNDQVLFIDQGNRPLFEDMTDSDCRDNAPRTIFIISMYADSQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 54 - protein, 157 aa, synthetic construct
YFGKLKSKLSVIRNLNDQVLFIDQGNRPLFEDMTDSDSRDNAPRTIFIISMYADSQPRGMAVTISVKSECISTLSSENKIISFKEMNPPDNIKDTKSDIIFFQRSVPGHDNKMQFESSSYEGYFLASEKERDLFKLILKKEDELGDRSIMFTVQNED

SEQ ID NO: 55 - protein, 157 aa, synthetic construct
YFGKLESKLSVIRNLNDQVLFIDQGNRPLFEDMTDSDSRDNAPRTIFIISMYADSQPRGMAVAISVKSECISTLSSENKIISFKEMNPPDNIKDTKSDIIFFQRSVPGHDNKMQFESSSYEGYFLASEKERDLFKLILKKEDELGDRSIMFTVQNED

SEQ ID NO: 56 - protein, 157 aa, synthetic construct
YFGKLKSKLSVIRNLNDQVLFIDQGNRPLFEDMTDSDSRDNAPRTIFIISMYADSQPRGMAVTISVKCEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 57 - protein, 157 aa, synthetic construct
YFGKLKSKLSVIRNLNDQVLFIDQGNRPLFEDMTDSDSRDNAPRTIFIISMYADSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 58 - protein, 157 aa, synthetic construct; identical to SEQ ID NO: 54

SEQ ID NO: 59 - protein, 157 aa, synthetic construct
YFGKLKSKLSVIRNLNDQVLFIDQGNRPLFEDMTDSDARDNAPRTIFIISMYADSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 60 - protein, 157 aa, synthetic construct
YFGKLKSKLSVIRNLNDQVLFIDQGNRPLFEDMTDSDQRDNAPRTIFIISMYADSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 61 - protein, 157 aa, synthetic construct
YFGKLKSKLSVIRNLNDQVLFIDQGNRPLFEDMTDSDARDNAPRTIFIISMYADSQPRGMAVTISVKCEKISTLSAENKIISFKEMNPPDNIKDTKSDIIFFQRSVPGHDNKMQFESSSYEGYFLACEKERDLFKLILKKEDELGDRSIMFTVQNED

SEQ ID NO: 62 - protein, 157 aa, synthetic construct
YFGKLKSKLSVIRNLNDQVLFIDQGNRPLFEDMTDSDARDNAPRTIFIISMYADSQPRGMAVTISVKCEKISTLSCENKIISFKEMNPPDNIKDTKSDIIFFQRSVPGHDNKMQFESSSYEGYFLAAEKERDLFKLILKKEDELGDRSIMFTVQNED

SEQ ID NO: 63 - protein, 157 aa, synthetic construct
YFGKLKSKLSVIRNLNDQVLFIDQGNRPLFEDMTDSDARDNAPRTIFIISMYADSQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 64 - protein, 157 aa, synthetic construct
YFGKLKSKLSVIRNLNDQVLFIDQGNRPLFEDMTDSDARDNAPRTIFIISMYADAQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 65 - protein, 157 aa, synthetic construct
YFGKLKSKLSVIRNLNDQVLFIDQGNRPLFEDMTDSDQRDNAPRTIFIISMYADAQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 66 - protein, 157 aa, synthetic construct
YFGKLKSKLSVIRNLNDQVLFIDQGNRPLFEDMTDSDCRDNAPRTIFIISMYADSQPRGMAVTISVKCEKISTLSCENKIISFAEMNPPDNIKDTKSDIIFFQRSVPGHDNKMQFESSSYEGYFLACEKERDLFKLILKKEDELGDRSIMFTVQNED

SEQ ID NO: 67 - protein, 157 aa, synthetic construct
YFGKLKSKLSVIRNLNDQVLFIDQGNRPLFEDMTDSDCRDNAPRTIFIISMYADSQPRGMAVTISVKCEKISTLSCENKIISFKEMNPPDNIKDTKSAIIFFQRSVPGHDNKMQFESSSYEGYFLACEKERDLFKLILKKEDELGDRSIMFTVQNED

SEQ ID NO: 68 - protein, 157 aa, synthetic construct
YFGKLESKLSIIRNLNDQVLFIDQGNRPLFEDMTDSDARDNAPRTIFIISGYADSQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 69 - protein, 157 aa, synthetic construct
YFGKLKSKLSIIRNLNDQVLFIDQGNRPLFEDMTDSDARDNAPRTIFIISGYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 70 - protein, 158 aa, synthetic construct
GYFGKLKSKLSIIRNLNDQVLFIDQGNRPLFEDMTDSDARDNAPRTIFIISGYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 71 - protein, 158 aa, synthetic construct
GYFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 72 - protein, 161 aa, synthetic construct
GGGGYFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 73-199 - skipped (127 entries)

SEQ ID NO: 200 - protein, 20 aa, synthetic construct
  VARIANT 1..20: note = This sequence may encompass 1-10 GS repeating units
GSGSGSGSGSGSGSGSGSGS

SEQ ID NO: 201 - protein, 30 aa, synthetic construct
  VARIANT 1..30: note = This sequence may encompass 1-10 GGS repeating units
GGSGGSGGSGGSGGSGGSGGSGGSGGSGGS

SEQ ID NO: 202 - protein, 40 aa, synthetic construct
  VARIANT 1..40: note = This sequence may encompass 1-10 GGGS repeating units
GGGSGGGSGGGSGGGSGGGSGGGSGGGSGGGSGGGSGGGS

SEQ ID NO: 203 - protein, 40 aa, synthetic construct
  VARIANT 1..40: note = This sequence may encompass 1-10 GGSG repeating units
GGSGGGSGGGSGGGSGGGSGGGSGGGSGGGSGGGSGGGSG

SEQ ID NO: 204 - protein, 50 aa, synthetic construct
  VARIANT 1..50: note = This sequence may encompass 1-10 GGSGG repeating units
GGSGGGGSGGGGSGGGGSGGGGSGGGGSGGGGSGGGGSGGGGSGGGGSGG

SEQ ID NO: 205 - protein, 50 aa, synthetic construct
  VARIANT 1..50: note = This sequence may encompass 1-10 GGGGS repeating units
GGGGSGGGGSGGGGSGGGGSGGGGSGGGGSGGGGSGGGGSGGGGSGGGGS

SEQ ID NO: 206 - protein, 15 aa, synthetic construct
GGGGSGGGGSGGGGS

SEQ ID NO: 207 - protein, 20 aa, synthetic construct
GGGGSGGGGSGGGGSGGGGS

SEQ ID NO: 208-299 - skipped (92 entries)

SEQ ID NO: 300 - protein, 133 aa, synthetic construct
  SITE 23: note = Norleucine
  SITE 39: note = Norleucine
  SITE 41: note = Homoserine
  SITE 42: note = Tyrosine residue modified with an azide-capped polyethylene glycol-9 group
  SITE 45: note = Tyrosine residue modified with an amino-capped polyethylene glycol-8 group
  SITE 46: note = Norleucine
  SITE 71: note = Homoserine
  SITE 104: note = Homoserine
APTSSSTKKTQLQLEHLLLDLQXILNGINNYKNPKLTRXLXXKFXXPKKATELKHLQCLEEELKPLEEVLXLAQSKNFHLRPRDLISNINVIVLELKGSETTFXCEYADETATIVEFLNRWITFSQSIISTLT

SEQ ID NO: 301 - protein, 133 aa, synthetic construct [sequence omitted]

SEQ ID NO: 302 - protein, 132 aa, synthetic construct
PTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 303 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 304 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTRXLXFKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 305 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTRXLXYKFYXPKKATELKHLQCLEEELKPLEEVLXYAQSKNFHLRPRDLISNINVIVLELKGSETTFXCEYADETATIVEFLNRWITFSQSIISTLT

SEQ ID NO: 306 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTRXLXFKFYXPKKATELKHLQCLEEELKPLEEVLXYAQSKNFHLRPRDLISNINVIVLELKGSETTFXCEYADETATIVEFLNRWITFSQSIISTLT

SEQ ID NO: 307 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTRXLXYKFYXPKKATELKHLQCLEEELKPLEEVLXGAQSKNFHLRPRDLISNINVIVLELKGSETTFXCEYADETATIVEFLNRWITFSQSIISTLT

SEQ ID NO: 308 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTRXLXYKFYXPKKATELKHLQCLEEELKYLEEVLXLAQSKNFHLRPRDLISNINVIVLELKGSETTFXCEYADETATIVEFLNRWITFSQSIISTLT

SEQ ID NO: 309 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTRXLXFKFYXPKKATELKHLQCLEEELKYLEEVLXLAQSKNFHLRPRDLISNINVIVLELKGSETTFXCEYADETATIVEFLNRWITFSQSIISTLT

SEQ ID NO: 310 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTYXLXYKFYXPKKATELKHLQCLEEYLKYLEEVLXLAQSKNFHLRPRDLISNINVIVLELKGSETTFXCEYADETATIVEFLNRWITFSQSIISTLT

SEQ ID NO: 311 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTRXLXFKFYXPKKATELKHLQCLEEYLKYLEEVLXLAQSKNFHLRPRDLISNINVIVLELKGSETTFXCEYADETATIVEFLNRWITFSQSIISTLT

SEQ ID NO: 312 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTRXLXFKFYXPKKATELKHLQCLEEYLKPLEEVLXLAQSKNFHLRPRDLISNINVIVLELKGSETTFXCEYADETATIVEFLNRWITFSQSIISTLT

SEQ ID NO: 313 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTRXLXFYFYXPKKATELKHLQCLEEELKPLEEVLXLAQSKNFHLRPRDLISNINVIVLELKGSETTFXCEYADETATIVEFLNRWITFSQSIISTLT

SEQ ID NO: 314 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTRXLXFKYYXPKKATELKHLQCLEEELKPLEEVLXLAQSKNFHLRPRDLISNINVIVLELKGSETTFXCEYADETATIVEFLNRWITFSQSIISTLT

SEQ ID NO: 315 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPYLTRXLXFKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 316 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEYLLLDLQXILNGINNYKNPKLTRXLXFKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 317 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEYLLLDLQXILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 318 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLYLQXILNGINNYKNPKLTRXLXFKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 319 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLYLQXILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 320 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPYLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 321 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTRXLXYKFYXPKKATELKHLQCLEEYLKPLEEVLXLAQSKNFHLRPRDLISNINVIVLELKGSETTFXCEYADETATIVEFLNRWITFSQSIISTLT

SEQ ID NO: 322 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTRXLXYKFYXPKKATELKHLQCLEEYLKYLEEVLXLAQSKNFHLRPRDLISNINVIVLELKGSETTFXCEYADETATIVEFLNRWITFSQSIISTLT

SEQ ID NO: 323 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTRXLXFKFYXPKKATELKHLQCLEEELKPLEEVLXLAQSKNFHLRPRDLISNINVIVLELKGSETTFXCEYADETATIVEFLNRWITFCQSIISTLT

SEQ ID NO: 324 - protein, 177 aa, synthetic construct
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 325 - protein, 154 aa, synthetic construct
MFHVSFRYIFGIPPLILVLLPVTSSDCHIKDKDGKAFGSVLMISINQLDKMTGTDSDCPNNEPNFFKKHLCDDTKEAAFLNRAARKLRQFLKMNISEEFNDHLLRVSDGTQTLVNCTSKEEKTIKEQKKNDPCFLKRLLREIKTCWNKILKGSI

SEQ ID NO: 326 - protein, 154 aa, synthetic construct
MFHVSFRYIFGIPPLILVLLPVTSSECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSI

SEQ ID NO: 327 - protein, 176 aa, synthetic construct
MFHVSFRYIFGIPPLILVLLPVASSDCDISGKDGGAYQNVLMVNIDDLDNMINFDSNCLNNEPNFFKKHSCDDNKEASFLNRASRKLRQFLKMNISDDFKLHLSTVSQGTLTLLNCTSKGKGRKPPSLSEAQPTKNLEENKSSKEQKKQNDLCFLKILLQKIKTCWNKILRGIKEH

SEQ ID NO: 328 - protein, 176 aa, synthetic construct
MFHVSFRYIFGIPPLILVLLPVASSDCDFSGKDGGAYQNVLMVSIDDLDNMINFDSNCLNNEPNFFKKHSCDDNKEASFLNRAARKLKQFLKMNISDDFKLHLSTVSQGTLTLLNCTSKGKGRKPPSLGEAQPTKNLEENKSLKEQRKQNDLCFLKILLQKIKTCWNKILRGITEH

SEQ ID NO: 329 - protein, 219 aa, Homo sapiens
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 330 - protein, 178 aa, Homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN